(19) United States
(12) Patent Application Publication
(10) Pub. No.: US 2019/0038622 A1
(43) Pub. Date: Feb. 7, 2019

(54) NEUROPROTECTIVE AGENTS FOR TREATMENT OF NEURODEGENERATIVE DISEASES

(71) Applicant: WAYNE STATE UNIVERSITY, DETROIT, MI (US)

(72) Inventor: Aloke K. Dutta, Troy, MI (US)

(21) Appl. No.: 16/154,301

(22) Filed: Oct. 8, 2018

Related U.S. Application Data
(63) Continuation-in-part of application No. 16/124,974, filed on Sep. 7, 2018.
Application No. 16/124,974 is a continuation-in-part of application No. 14/648,413, filed on May 29, 2015, now Pat. No. 10,125,127.
(60) Provisional application No. 61/731,096, filed on Nov. 29, 2012.

(51) Int. Cl. A61K 31/497

(52) U.S. Cl. CPC A61K 31/497; A61K 31/428

(57) ABSTRACT

A compound having formula IA is useful for treating a neurodegenerative disease:

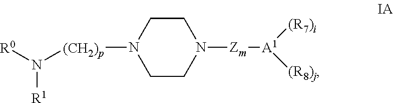

or a pharmaceutically acceptable salt or ester thereof, where R⁰, R¹, R², R⁷, R⁸ are delineated substituents; A¹ is a C₆₋₁₂ aryl group, C₅₋₁₂ heteroaryl group, substituted 3-hydroxypyridin-4(1H)-one,

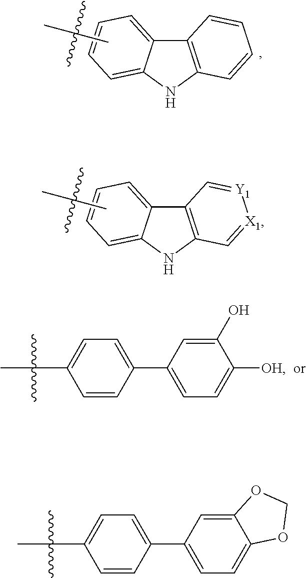

with i hydrogen atoms replaced with R⁷ and j hydrogen atoms replaced with R⁸; p is an integer from 1 to 6; X¹, Y¹ are each independently CH or N; M is absent or a divalent linking moiety in which Z is repeated m times; m is an integer from 0 to 5; i, j are each independently 0, 1, 2, or 3; and o is 0, 1, 2, 3, or 4.

### CROSS-REFERENCE TO RELATED APPLICATIONS

This application is a continuation-in-part of U.S. Ser. No. 16/124,974 filed Sep. 7, 2018 which is a continuation-in-part of U.S. Ser. No. 14/648,413 filed May 29, 2015 which is the U.S. national phase of PCT Application No. PCT/US2013/072253 filed Nov. 27, 2013, which claims the benefit of U.S. Ser. No. 61/731,096 filed Nov. 29, 2012, the disclosures of which are incorporated in their entirety by reference herein.

### STATEMENT REGARDING FEDERALLY SPONSORED RESEARCH OR DEVELOPMENT

The invention was made with Government support under Contract No. NS047198 awarded by the National Institutes of Health/National Institute of Neurological Disorders and Stroke. The Government has certain rights to the invention.

### TECHNICAL FIELD

The present invention relates to compounds for treating neurodegenerative diseases.

### BACKGROUND OF THE INVENTION

Dopaminergic receptor systems have been targeted for the development of pharmacotherapeutic agents for a number of CNS related disorders, including drug addiction, schizophrenia, depression, and Parkinson's disease (PD). Dopamine (DA) receptor agonists have been employed more extensively in the treatment of Parkinson's disease than any other type of pharmacotherapy. Levodopa (L-dopa), the immediate precursor to endogenous DA, is the current gold-standard treatment option for PD. DA receptors belong to the family of transmembrane proteins known as G-protein-coupled receptors (GPCRs). DA receptors are widely distributed in the CNS, are also present in the periphery, and are divided into five subtypes. On the basis of the stimulatory action on adenylyl cyclase, D1 and D5 are grouped together as D1 type. D2-D4 receptors are classified as D2 type because of their inhibitory action on adenylyl cyclase activity. Interestingly, the D3 receptor was found to have a distribution in the brain that is somewhat different from that of the D2 receptor. The highest levels of D3 receptor expression were found to be in the limbic region of the brain, while D2 receptor expression is most dense in the striatum of the midbrain. D2 and D3 receptor subtypes occur post- and presynaptically. In the latter location they function as autoreceptors that regulate DA synthesis, metabolism, and release. It is noteworthy that D2 and D3 receptor subtypes share 50% overall amino acid sequence homology and 75-80% in their agonist binding sites. As a result, development of ligands selective for either subtype is a challenging task.

Parkinson's disease (PD) is a progressive, neurodegenerative disorder that results from the death of DA-producing cells in the substantia nigra region of the midbrain. Common symptoms include resting tremor, muscular rigidity, bradykinesia, postural instability, and cognitive psychiatric complications. Although the etiology of PD is not yet clear and may be multifactorial, oxidative stress and mitochondrial dysfunction are thought to play a central role in the pathology of the disease. Recent studies on various genetic mutations have provided new insights into the disease process. Oxidative stress has been strongly implicated in midbrain dopaminergic cell death. Toxicity from endogenous and exogenous origins, caused by oxidative mechanisms, has been implicated as a fundamental process in progressive nigral cell loss. Along with motor fluctuations and wearing off after long-term treatment, side effects associated with L-dopa treatment and the eventual oxidation of DA derived from L-dopa have been speculated to produce further oxidative stress.

In addition, α-synuclein, a presynaptic protein involved in fibrillization, has been implicated in the pathogenesis of PD. A recent report demonstrated that in cultured human dopaminergic neurons, accumulation of α-synuclein induces apoptosis in the presence of DA and reactive oxygen species. Furthermore, an interaction between calcium, cytosolic DA, and α-synuclein has been implicated in the loss of DA neurons in the substantia nigra. In this case, DAdependent neurotoxicity is mediated by a soluble protein complex containing α-synuclein. Therefore, α-synuclein, together with oxidized DA, could have synergistic effects in terms of disease susceptibility and progression.

Accordingly, there is a need for dopamine improved D2/D3 agonist molecules, and in particular, for improved D2/D3 agonist molecules with a capacity to bind to iron.

### SUMMARY OF THE INVENTION

In at least one embodiment, the present invention solves one or more problems of the prior art by providing a compound having formula IA for treating a neurodegenerative disease:

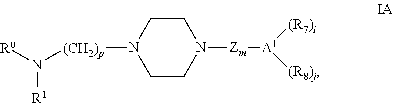

or a pharmaceutically acceptable salt or ester thereof,
wherein

R⁰ is an optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, C₆₋₁₀ aryl, or

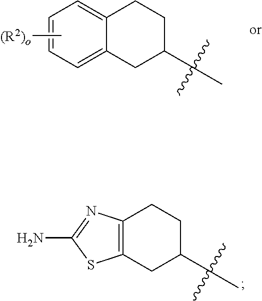

R¹ is an optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, or C₆₋₁₀ aryl;

R² are hydroxyl, C₁₋₄ alkyl, C₁₋₄ alkoxyl, C₁₋₈ alkyl, C₂₋₈ alkenyl, C₂₋₈ alkynyl, C₄₋₈ cycloalkyl, C₄₋₈ cycloalkenyl; C₆₋₁₀ aryl, —NR³_{q} or C₁₋₁₀ hydrocarbon groups optionally containing one or more O, N, S, or Se heteroatoms where R³ individually are H or organyl groups and q is 2 or 3, with the proviso that when q is 3, the group bears a positive formal charge;

R⁷, R⁸ are each independently, H, hydroxyl, oxo, C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo, C₁₋₄ aldehyde, or —NR⁴_{q} where R⁴ is H, C₁₋₈ alkyl, C₂₋₈ alkenyl, C₄₋₈ cycloalkyl, 3H-1,2-dithiole-3-thione, C₄₋₈ cycloalkenyl, or C₆₋₁₀ aryl;

A¹ is a C₆₋₁₂ aryl group, C₅₋₁₂ heteroaryl group, substituted 3-hydroxypyridin-4(1H)-one,

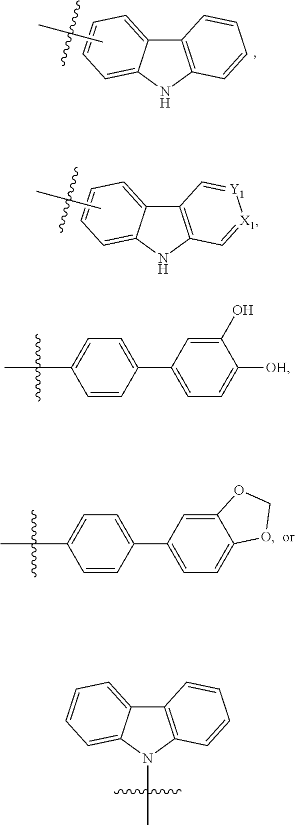

with i hydrogen atoms replaced with R⁷ and j hydrogen atoms replaced with R⁸;

p is an integer from 1 to 6;

X¹, Y¹ are each independently CH or N;

Zₘ is absent or a divalent linking moiety in which Z is repeated m times;

m is an integer from 0 to 5;

i, j are each independently 0, 1, 2, or 3; and

o is 0, 1, 2, 3, or 4.

### BRIEF DESCRIPTION OF THE DRAWINGS

Exemplary embodiments of the present invention will become more fully understood from the detailed description and the accompanying drawings, wherein:

FIGS. 1-6 provide compounds having formula I;

FIGS. 7-10 provide examples of compounds having formula II;

FIG. 11 provides a synthetic scheme for compounds having formula II;

FIG. 12 provides a synthetic scheme for compounds having formula I;

FIG. 13 provides a synthetic scheme for compounds having formula II;

FIG. 14 provides a synthetic scheme for compounds having formula II;

FIG. 15 provides a synthetic scheme for compounds having formula II;

FIG. 16 provides a synthetic scheme for compounds having formula II;

FIG. 17 provides a synthetic scheme for compounds having formula II;

FIG. 18 provides a synthetic scheme for compounds having formula I;

FIG. 19. Structures of carbazole compounds and D2/D3 receptor agonists;

FIG. 20. Effects of different drugs upon reserpine (5.0 mg/kg, sc, 18 h pretreatment) induced hypolocomotion in rats. Each point represents the mean±SEM for three rats. Horizontal activity was measured as described in Experimental Section. Representation of horizontal locomotor activity is at discrete 30 min intervals after the administration of (−)-81b (D-636) (10 μMol/kg), (−)-85a (D-653) (10 μMol/kg), (−)-95c (D-656) (10 μMol/kg), and ropinirole (10 μMol/kg) compared to control rats, 18 h after reserpine treatment. Differences among treatments were significant by one-way ANOVA analysis (F (4, 60)=8.861; (P<0.0001)). Dunnett's analysis following ANOVA revealed that the effect of the compounds (−)-81b, (−)-85a and (−)-85c (p<0.0001) was significantly different when compared to reserpine control;

FIG. 21A-C. Detection of intracellular ROS using DCFDA-based fluorescence assay: PC12 cells were pretreated with different doses of (−)-81b (D-636) (A), (−)-85a (D-653) (B), and (−)-85c (D-656) (C) for 24 h followed by treatment with DCFDA (20 μM, 2% serum) for 30 min. The DCFDA containing media was removed, and replaced with fresh media, followed by treatment with 75 μM 6-OHDA, and incubated for 1 h. Data represents mean±SDs of three independent experiments in four to six replicates. One-Way ANOVA analysis followed by Tukey's multiple comparison post hoc test was performed. (*P<0.05, **P<0.01, ***P<0.001, and ****P<0.0001 compared to the 6-OHDA, ^{####}P<0.0001 compared to the control);

FIG. 22A-F. Dose-dependent effect on the viability of PC12 cells by 24 h pretreatment of compounds (−)-81b (D-636), (−)-85a (D-653), and (−)-85c (D-656) at various concentrations followed by single treatment of 75 μM 6-OHDA. (a, c and e) Dose-dependent effect of (−)-81b, (−)-85a, and (−)-85c on cell viability, respectively. (b, d and f) PC12 cells were pretreated with varying concentrations of (−)-81b, (−)-85a, and (−)-85c, respectively for 24 h followed by treatment with 7 μM 6-OHDA for another 24 h. The results shown are mean±SEM of three independent experiments performed in seven to eight replicates. One way ANOVA analysis F (7, 54)=21.64, p<0.0001 for (−)-81b; F (7, 56)=167.5, p<0.0001 for (−)-85a; and F (7, 56)=39.78, p<0.0001 for (−)-85c. ANOVA was followed by Tukey's multiple comparison post hoc test (****p<0.0001, ***p<0.001, **p<0.01 compared to control); (correct the label as shown here in the corresponding fig file)

FIG. 23A-B. Effect of 81b (D-636) and 85a (D-653) on the aggregation of α-syn induced by seeding with 0.5% PFFs: 1.25 mg/mL α-syn was incubated with 0.5% PFFs for a period of 30D without shaking in the presence of 81b or 85a at a concentration of 172.9 μM. Fibrillation was measured by ThT assay at 30D (A). Values are represented in terms of % 0D Synuclein. Viability of PC12 cells was measured by MTT assay after 24 h treatment with α-syn seeded samples collected at 30D (B). Values are represented in terms of % control. Data values shown are means±SD of three independent experiments. One-way ANOVA analysis followed by Tukey's multiple comparison post hoc test was performed, **p≤0.01, ****p≤0.0001 compared to Syn +0.5%-0D; ####p≤0.0001 compared to Syn +0.5%-30D; (correct the label as shown here in the corresponding fig file)

FIG. 24. Table 3. Inhibition constants determined by competition experiments assessing [³H]spiroperidol binding to cloned rat D_{2L} and D₃ receptors expressed in HEK-293 cells^{α}. cLogP values are calculated by using SwissADME data base and tPSA values are calculated by using ChemDraw.

FIG. 25. Table 4. Stimulation of [³⁵S]GTPγS binding to cloned human D₂ and D₃ receptors expressed in CHO cells^{α};

FIG. 26 provides a synthetic scheme for compounds having formula I;

FIG. 27 provides a synthetic scheme for compounds having formula I;

FIG. 28 provides a synthetic scheme for compounds having formula I; and

FIG. 29 provides a synthetic scheme for compounds having formula I.

FIGS. 30A and 30B. Effect of D-687, D-688 and D-690 on aggregation and disaggregation of Aβ₁₋₄₂.A) Inhibition of Aβ₁₋₄₂ oligomer formation by the drugs: ThT fluorescence signals of Aβ₁₋₄₂ (10 μM) with or without incubation with the drugs (20 μM) for 24 h. The ThT fluorescence of Aβ₁₋₄₂ at 0 h was taken as 100%. ThT fluorescence with respect to time is plotted. B) Dissociation of Aβ₁₋₄₂ fibrils by the drugs: Aβ₁₋₄₂ fibrils (10 μM) were incubated with the drugs (20 μM) for a period of 24 h. The ThT fluorescence of Aβ₁₋₄₂ aggregates at 0 h was considered as 100%. ThT fluorescence with respect to time is plotted.

### DETAILED DESCRIPTION OF THE INVENTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; all R groups and X or Y groups (as a substituent attached to a carbon e.g., replacing a potential C—H bond) include H, C₁₋₁₀ alkyl, C₂₋₁₀ alkenyl, halo, aryl, heteroaryl, and the like; all X, Y, Z symbols are X and Y may be N, CH, NR, O, S, and Se where R is hydrogen, alkyl, or aryl; all lower case letters (e.g., i, j, o, p, etc) are integers 0, 1, 2, 3, 4, 5, or 6; the first use of a symbol carries through to subsequent uses unless defined to the contrary; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

In a variation, the term “organyl group” means any organic substituent group, regardless of functional type, having one free valence at a carbon atom, e.g. methyl, ethyl, propyl, butyl, pyridinyl, 4-pyridylmethyl, and the like.

In a variation, the term “alkyl” means a straight or branched hydrocarbon radical having from 1 to 10 carbon atoms and includes, for example, methyl, ethyl, n-propyl, isopropyl, n-butyl, sec-butyl, isobutyl, tert-butyl, n-pentyl, n-hexyl, n-heptyl, n-octyl, and the like.

In a variation, the term “alkanediyl” means a straight or branched hydrocarbon diradical having from 1 to 10 carbon atoms formed by removing 2 hydrogen atoms from an alkane.

In a variation, the term “alkoxy” and “thioalkoxy” are O-alkyl or S-alkyl of from 1 to 8 carbon atoms as defined above for “alkyl”.

In a variation, the term “cycloalkyl” means a saturated hydrocarbon ring having 3 to 8 carbon atoms and includes, for example, cyclopropyl, cyclobutyl, cyclopentyl, cyclohexyl, cycloheptyl, cyclooctyl, and the like.

In a variation, the term “alkenyl” means a straight or branched unsaturated hydrocarbon radical having from 2 to 12 carbon atoms and includes, for example, ethenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, 1-dodecenyl, and the like.

In a variation, the term “alkenediyl” means a straight or branched hydrocarbon diradical having from 2 to 12 carbon atoms formed by removing 2 hydrogen atoms from a C₂₋₁₂ alkene.

In a variation, the term “alkynyl” means a straight or branched triple bonded unsaturated hydrocarbon radical having from 2 to 12 carbon atoms and includes, for example, ethynyl, 2-propynyl, 3-butynyl, 4-pentynyl, 5-hexynyl, 6-heptynyl, 7-octynyl, 8-nonynyl, 9-decynyl, 10-undecynyl, 11-dodecynyl, and the like.

In a variation, the term “alkynediyl” means a straight or branched hydrocarbon diradical having from 2 to 12 carbon atoms formed by removing 2 hydrogen atoms from a C₂₋₁₂ alkyne.

In a variation, the term “cycloalkylalkyl” means a saturated hydrocarbon ring attached to an alkyl group wherein alkyl is as defined above. The saturated hydrocarbon ring contains from 3 to 12 carbon atoms. Examples of such are cyclopropylmethyl, cyclopentylmethyl, cyclohexylmethyl, adamantylmethyl and the like.

In a variation, the term “heterocycloalkyl” means a saturated hydrocarbon ring having 3 to 8 carbon atoms in which 1 or more carbon atoms are replaced by N, S, O, Se, etc. Examples includes 2- or 3-tetrahydrothieno, 2- or 3-tetrahydrofurano, 2- or 3-pyrrolidino, 2-, 4-, or 5-thiazolidino, 2-, 4-, or 5-oxazolidino, 2-, 3-, or 4-piperidino, N-morpholinyl or N-thiamorpholinyl.

In a variation, the term “aryl” means an aromatic radical such as a phenyl group, a naphthyl group, a phenyl group substituted by 1 to 4 substituents selected from alkyl as defined above, alkoxy as defined above, thioalkoxy as defined above, hydroxy, halogen, trifluoromethyl, amino, alkylamino as defined above for alkyl, dialkylamino as defined for alkyl, N-acetylamino, cyano —SO₂NH₂, or nitro, or a naphthyl group substituted by 1 to 4 substituents as defined above for a phenyl group substituted by 1 to 4 substituents. In a refinement, aryl is a C₆₋₁₄ aryl.

In a variation, the term “heteroaryl” means a C₅₋₁₃ heteroaromatic radical such as 2- or 3-thienyl; 2- or 3-furanyl; 1-, 2- or 3-pyrrolyl; 1-, 2-, 4-, or 5-imidazolyl; 1-, 3-, 4-, or 5-pyrazolyl; 2-, 4-, or 5-thiazolyl; 3-, 4-, or 5-isothiazolyl; 2-, 4-, or 5-oxazolyl; 3-, 4-, or 5-isoxazolyl; 1-, 3-, or 5-1,2,4-triazolyl; 1-, 2-, 4-, or 5-1,2,3-triazolyl; 1- or 5-tetrazolyl; 4-, or 5-1,2,3-oxadiazolyl; 3-, or 5-1,2,4-oxadiazolyl; 2-1,3,4-oxadiazolyl; 2-1,3,4-thiadiazoyl; 2-1,3,5-triazinyl; 3-pyridinyl; 3-, 4-, or 5-pyridazinyl; 2-pyrazinyl; 2-, 4-, or 5-pyrimidinyl; unsubstituted or substituted by 1 to 2 substituents selected from NH₂, OH, S, halogen as defined hereinafter, alkyl as defined above, or alkoxy as defined above. In a refinement, heteroaryl is a C₅₋₁₀ heteroaryl.

In a variation, the term “halogen” or “halo” means fluorine, chlorine, bromine, or iodine.

It should be appreciated that each C—H bond in the formulae set forth herein can be substituted. For example, each C—H bond can be substituted by halo, cyano, nitro, hydroxyl, C₁₋₁₀ alkyl, C₁₋₈ alkoxyl, C₆₋₁₄ aryl, C₅₋₁₃ heteroaryl, NH₂SO₂R, CF₃, arylsulfonyl, arylsulfonamide, o-OCH₃, pyridinyl, bipyridinyl, phenyl, chloro, bromo, fluoro, and the like. Such substituted C—H bonds can be symbolized by C—R^{z} where z is an integer from 1 to 100 that has not already been used as a subscript or subscript for an R group.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100.

Abbreviations:

“h” is hour;

“s” is seconds;

“rt” is room temperature;

“et” is ethyl;

“me” is methyl;

“BOC” is Di-tert-butyl dicarbonate;

“TBDMS” is tert-butyldimethylsilyl;

“THF” is tetrahydrofuran;

“DMSO” is dimethylsulfoxide;

“DMAP” is 4-dimethylaminopyridine;

“TFA” is trifluoroacetic acid;

“DCE” is 1,2-dichloroethane;

“tol” is tolyl;

“DIBALH” is diisobutylaluminium hydride;

“DCM” is dichloromethan;

“DMF” is dimethylformamide;

“TBAF” is tetra-n-butylammonium fluoride;

“NaHMDS” is sodium bis(trimethylsilyl)amide; and

“PCC” is pyridinium chlorochromate.

“Cs₂CO₃” means cesium carbonate.

“BINAP” means 2,2′-bis(diphenylphosphino)-1,1′-binaphthyl

“Pd(OAc)2” meanspalladium(II) acetate.

“TBAF” means tetrabutylammonium fluoride.

“SO₃.py” means sulfur trioxide pyridine.

“NaBH(OAc)3” means sodium triacetoxyborohydride.

“GTPγS” means guanosine 5′-O-[gamma-thio]triphosphate.

“5-OH-DPAT” means 5-hydroxy-2-(dipropylamino)tetralin.

“CHO” means chinese hamster ovary.

“HEK” means human embryonic kidney.

“L-DOPA” means (S)-(3,4-dihydroxyphenyl) alanine.

“i.p.” means intraperitoneal.

“s.c.” means subcutaneous.

“PD: Parkinson's disease.

“DA” means dopamine.

In at least one embodiment, the present invention provides a compound having formula I for treating a neurodegenerative and other related CNS diseases:

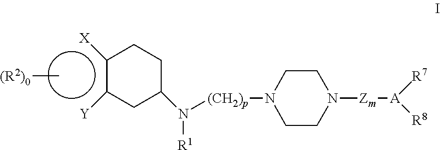

R¹ is a C₁₋₁₂ organyl group, preferably selected from among optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, or C₆₋₁₀ aryl, each of the foregoing alkyl, alkenyl, alkynyl, cycloalkyl, etc. groups optionally halo substituted, preferably fluoro and/or chloro substituted, or substituted by C₁₋₄ alkoxy, C₁₋₈ alkoxy C₁₋₄ acyloxy, C₁₋₄ acyl, —C(O)—R⁴, or —R⁵—NH—SO₂—NR⁴ᵣ, —R⁵—NH—C(O)—R⁴, —R⁵—NR⁴ᵣ, or —R⁵—Ar where R⁴ is H or C₁₋₁₂ organyl groups, preferably H, C₁₋₈ alkyl, C₂₋₈ alkenyl, C₄₋₈ cycloalkyl, C₄₋₈ cycloalkenyl, or C₆₋₁₀ aryl; and R⁵ is C₁₋₈ alkenyl, and r is 2 or 3, with the proviso that when r is 3, the nitrogen of the NR⁴ᵣ group will bear a positive formal charge. When there is a formal charge, the compound includes a suitable negatively charged counter ion such as chloride, bromide, and the like. Ar is a C₆₋₁₀ aryl ring system, preferably a C₆₋₁₀ aryl ring system, optionally including one or more heteroatoms or a C₅₋₁₀ C₅₋₁₂ heteroaryl. Preferably Ar is phenyl, thienyl, pyridyl, biphenyl, or naphthyl each optionally substituted with CN, halo, C₁₋₈ alkyl, C₂₋₈ alkenyl, C₄₋₈ cycloalkyl, C₄₋₈ cycloalkenyl, or C₆₋₁₀ aryl;

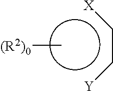

is a C₆₋₁₂ aromatic ring system, C₅₋₁₂ heteroaromatic ring system and/or an optionally heterocyclic ring system containing 5 to 12 ring atoms and up to three heteroatoms individually selected from the group consisting of N, O, S, and Se. When the ring system includes 0 heteroatoms, the ring system is formally a C₅₋₁₂ carbon ring system (e.g., a cycloalkyl). In a refinement, this rings systems are fused to each other or to an aromatic ring. They also are linked together or to an aromatic ring. In another variation, this moiety is an optionally heterocyclic ring system containing 5 or 6 ring atoms and up to three heteroatoms individually selected from the group consisting of N, O, S, and Se. This C₆₋₁₂ aromatic ring system, C₅₋₁₂ heteroaromatic ring system and/or an optionally heterocyclic ring system is optionally substituted by o R² groups, where o is 0, 1, 2, 3, or 4, the upper limit bounded by the number of available substituent sites. In a variation, X and Y may be N, CH, NR, O, S, and Se where R is hydrogen, alkyl, or aryl;

R² are C₁₋₁₂ organyl groups, hydroxyl, nitro, or —NR⁴_{q} where R⁴ is H, C₁₋₈ alkyl, C₂₋₈ alkenyl, C₄₋₈ cycloalkyl, C₄₋₈ cycloalkenyl, or C₆₋₁₀ aryl. In a variation, R² are C₁₋₁₀ hydrocarbon groups optionally containing one or more O, N, S, or Se heteroatoms. In another variation, R² are selected from among C₁₋₄ alkyl, C₁₋₄ alkoxyl, C₁₋₈ alkyl, C₂₋₈ alkenyl, C₂₋₈ alkynyl, C₄₋₈ cycloalkyl, C₄₋₈ cycloalkenyl; C₆₋₁₀ aryl, —NR³_{q} where R³ individually are H or organyl groups. For example, R³ may be H, C₁₋₈ alkyl, C₁₋₈ alkoxyl, C₂₋₈ alkenyl, C₂₋₁₀ alkynyl, C₄₋₈ cycloalkyl, C₄₋₈ cycloalkenyl, C₆₋₁₀ aryl and q is 2 or 3, with the proviso that when q is 3, the group bears a positive formal charge; —NH—C(O)—R³, —NH—C(O)—NR⁴₂, and related compounds wherein the hydrocarbon groups in each case may optionally be substituted with —CN, C₁₋₄ alkyl, C₁₋₈ alkyl, —OR³, —OH, halo, particularly fluoro and/or chloro, —CF₃, and the like. Two R² may also together form an alicyclic or aromatic fused five or six membered ring, optionally containing heteroatoms O, N, S, or Se. R³ may also be arylsulfonyl, preferably 4-chlorophenylsulfonyl, 3,4-dichlorophenylsulfonyl; 4-(trifluoromethyl)phenylsulfonyl; X⁴—Ar—SO₂ where X⁴ is an electron withdrawing or electron donating substituent and Ar is a C₆₋₁₀ aromatic or C₅₋₁₀ heteroaromatic moiety; or keto, preferably phenylketo, 4-(trifluoromethyl)phenylketo, or aceto;

R⁷, R⁸ are each independently, hydrogen (H), hydroxyl, oxo (i.e., carbonyl), C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo (e.g. fluoro or chloro), C₁₋₄ aldehyde, or —NR⁴_{q} where R⁴ is H, C₁₋₈ alkyl, C₂₋₈ alkenyl, C₄₋₈ cycloalkyl, C₄₋₈ cycloalkenyl, or C₆₋₁₀ aryl and like groups where R⁴ is H or C₁₋₁₂ organyl groups, preferably H, C₁₋₈ alkyl, C₂₋₈ alkenyl, C₄₋₈ cycloalkyl, C₄₋₈ cycloalkenyl, 3H-1,2-dithiole-3-thione or C₆₋₁₀ aryl. In a variation, R⁷, R⁸ are not hydroxyl;

A is a C₆₋₁₂ aryl group, C₅₋₁₂ heteroaryl group, or an optionally substituted 3-hydroxypyridin-4(1H)-one. In one variation, A contains one or more heteroatoms. In another variation, A may be directly bonded to a nitrogen atom of the piperazinyl group. In still another variation, A is an C₆₋₁₂ group consisting of 1 to 4 rings, may contain heterocyclic rings, optionally fused, optionally linked, and optionally substituted by C₁₋₄ alkyl, C₁₋₄ alkoxyl, C₁₋₄ alkyl, C₁₋₈ alkoxyl, C₂₋₄ alkenyl, C₂₋₁₀ alkynyl, C₅₋₇ cycloalkyl, C₅₋₇ cycloalkenyl, halo, preferably fluoro or chloro, C₁₋₄ aldehyde, —NR⁴_{q} and like groups where R⁴ is H or organyl groups, preferably H, C₁₋₈ alkyl, C₂₋₈ alkenyl, C₄₋₈ cycloalkyl, C₄₋₈ cycloalkenyl, or C₆₋₁₀ aryl; and q is 2 or 3, with the proviso that when q is 3, the group bears a positive formal charge. It should be noted that unsaturated groups such as alkenyl and cycloalkenyl include multiply unsaturated groups such as alkadienyl and cycloalkadienyl. Alkyl and cycloalkyl groups herein also include aryl-substituted alkyl and cycloalkyl groups, while aryl groups also include alkyl and cycloalkyl-substituted aryl groups. In a variation, A is optionally substituted thienyl, pyridyl, bipyridyl, phenyl, biphenylyl, or naphthyl, more preferably phenyl and biphenyl, quinolone, indole, and isoquinoline. Preferred substituents include C₁₋₄ alkyl, C₁₋₈ alkyl, —CN, halo, C₁₋₈ alkoxyl, C₁₋₄ alkoxyl, and NH₂SO₂R³, CF₃, arylsulfonyl, arylsulfonamide, etc., more preferably o-OCH₃, 2,3-dichloro, and p-NHSO₂CH₃;

p is an integer from 1 to 6; and

Zₘ is absent or a divalent linking moiety in which Z is repeated m times. Examples of Z include —CH₂—, —CHOHCH₂—, —CHOHCH₂CH₂—, —CHOHCH₂CH₂CH₂—, —CO—, —N—CH₂— or —N—CO—, where m is an integer from 0 to 5. In another variation, m is an integer from 0 to 2. In a refinement, m is 0, 1, 2, 3, 4, or 5 and p is 1, 2, 3, 4, 5, or 6. In a refinement, Zₘ is —(CH₂)ₙ—, —CHOH(CH₂)ₙ—, —(CH₂)ₙCO—, —(CH₂)ₙNCO(CH₂)ₖ—, C₁₋₁₀ carboximido, C₁₋₁₀ alkyl (i.e., an C₁₋₁₀ alkanediyl), C₂₋₁₀ alkenyl (i.e., an C₂₋₁₀ alkanediyl), (CH₂)ₙCH═CH(CH₂)ₖ, (CH₂)ₙCC(CH₂)ₖ, C₂₋₁₀ alkynyl (i.e., an C₂₋₁₀ alkynediyl), where n and k are each independently integers from 0 to 8 (e.g., 0, 1, 2, 3, 4, 5, 6, 7, or 8). In another refinement, Zₘ is a combination of each of the specific examples set forth herein.

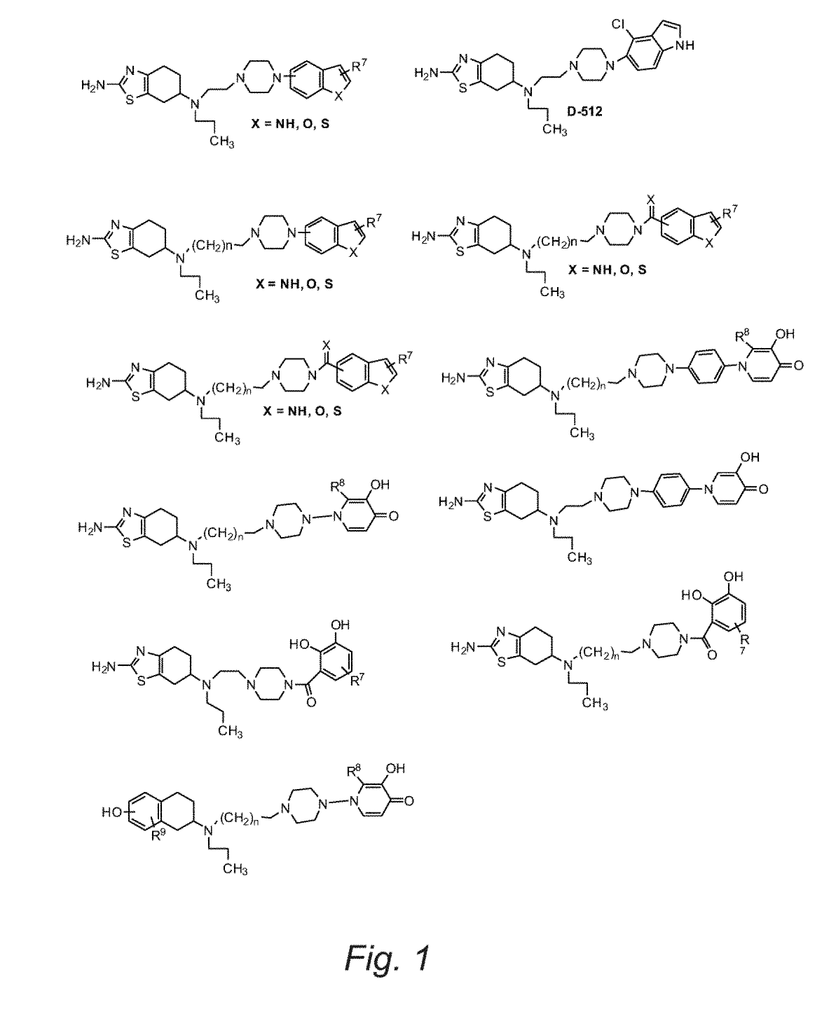

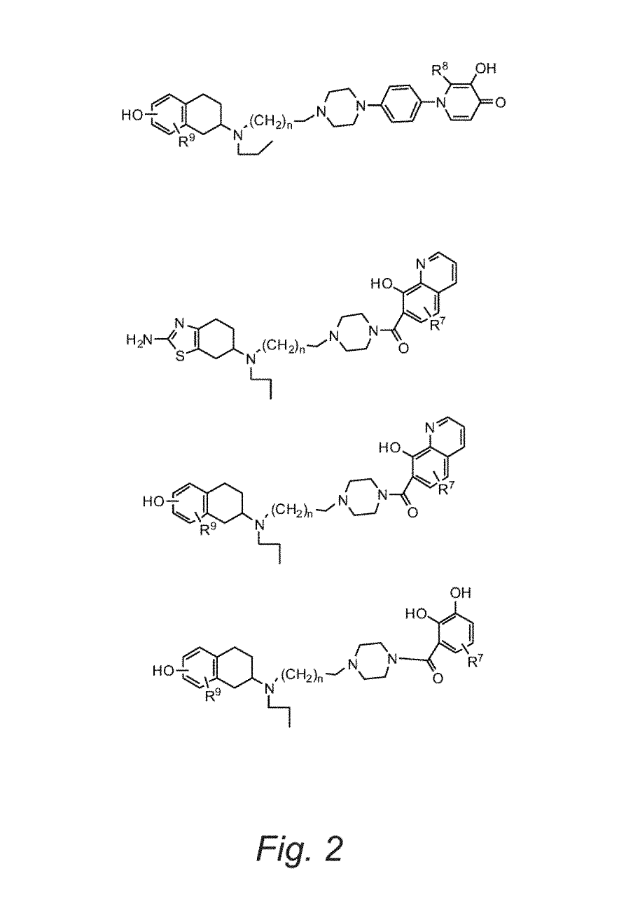

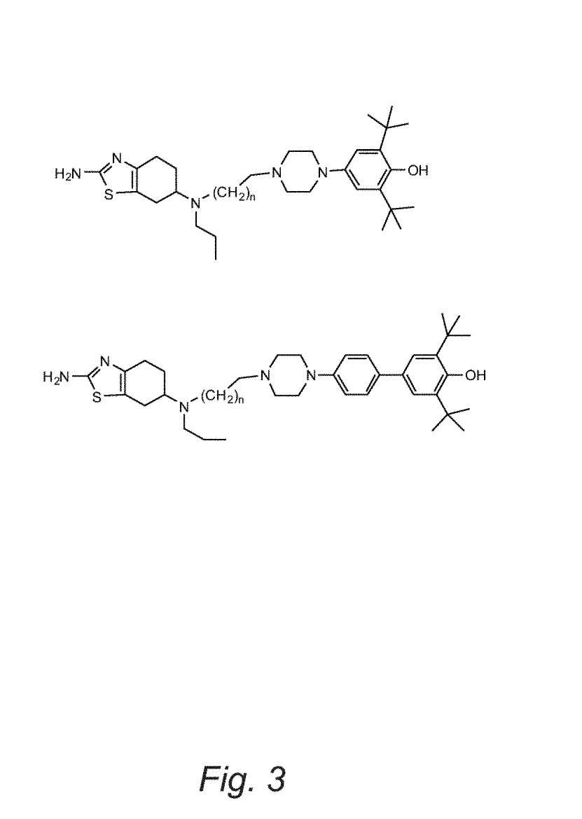

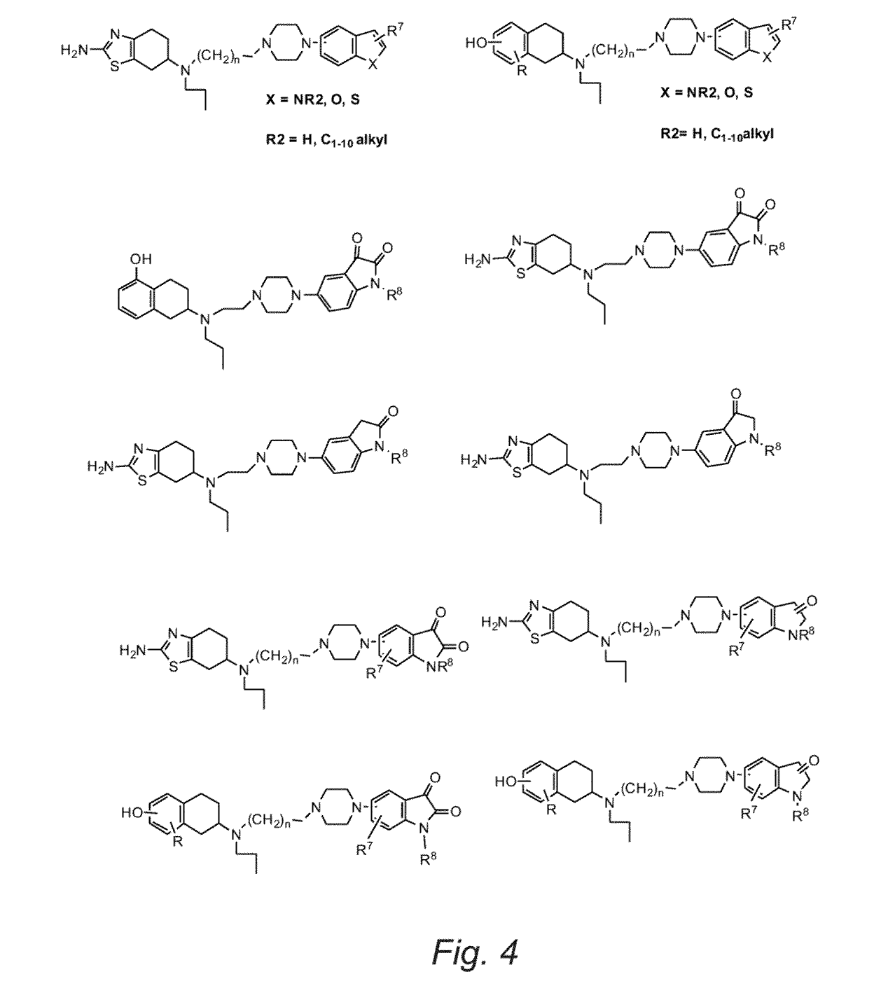

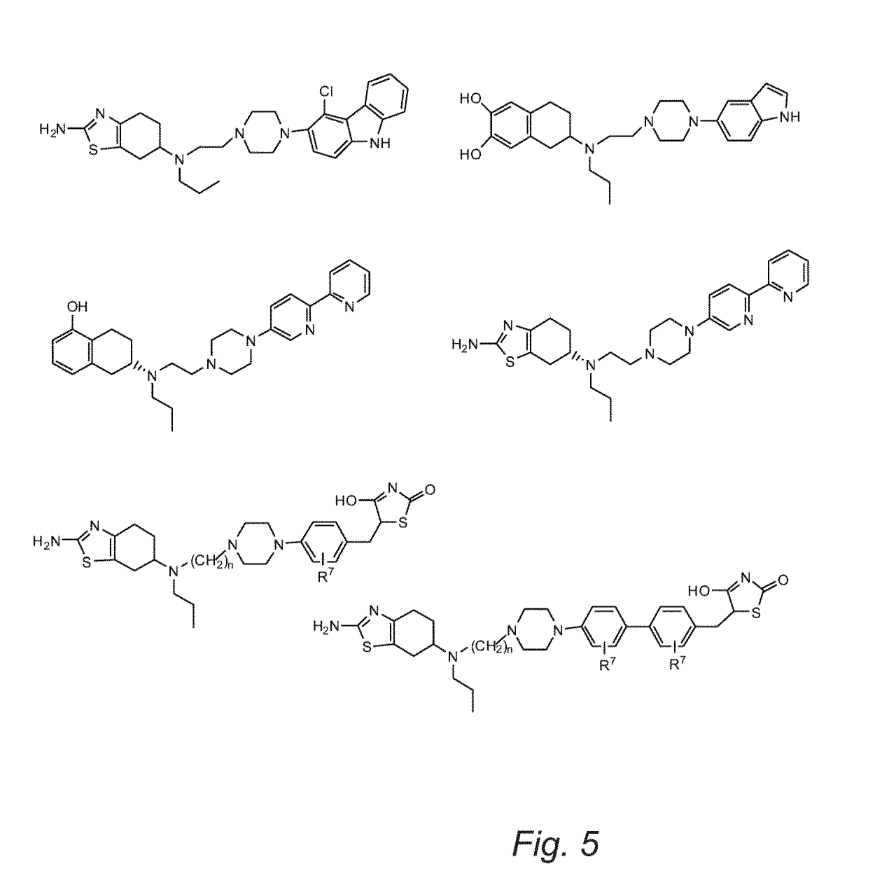

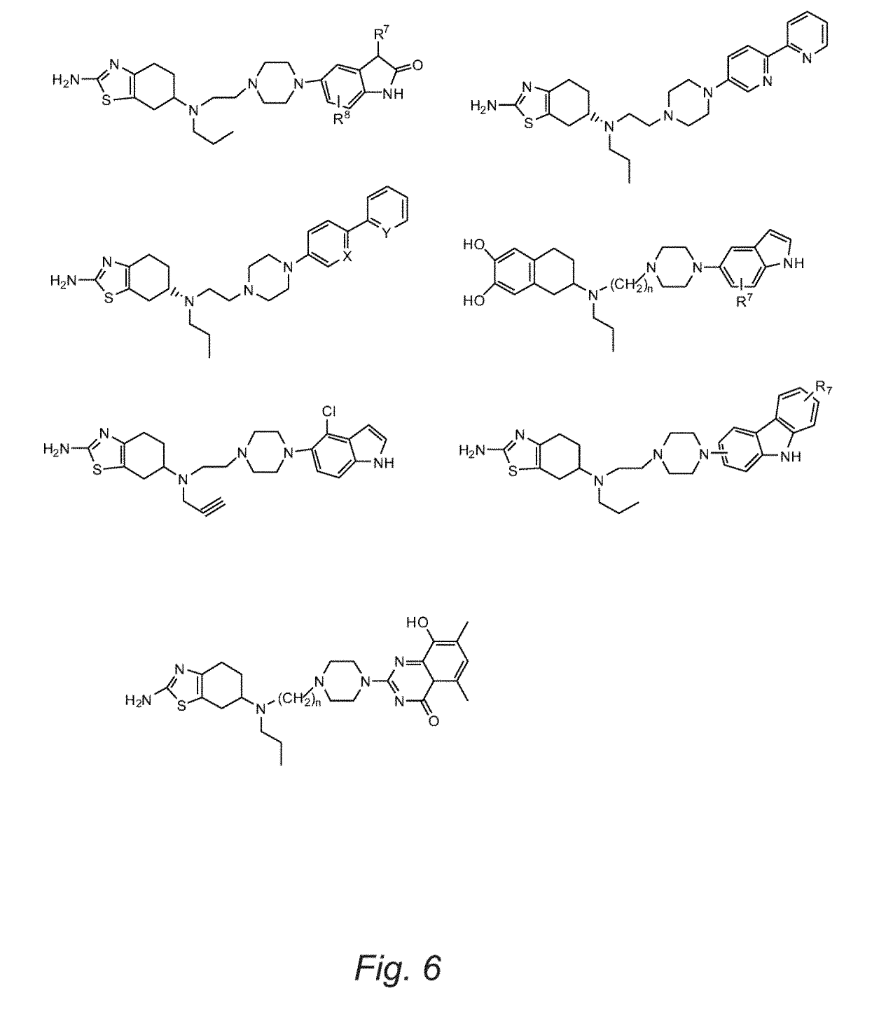

FIGS. 1-6 provide examples of compound falling within the present embodiment. In these Figures, n is 0 to 8 and more particularly, n is 0, 1, 2, 3, 4, 5, or 6. R⁷, R⁸ are as set forth above. In particular, R⁷, R⁸ and R⁹ is hydrogen (H), hydroxyl, C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo, preferably fluoro or chloro, C₁₋₄ aldehyde, —NR⁴_{q} and like groups where R⁴ is H or organyl groups, preferably H, C₁₋₈ alkyl, C₂₋₈ alkenyl, C₂₋₁₀ alkynyl, C₄₋₈ cycloalkyl, C₄₋₈ cycloalkenyl, or C₆₋₁₀ aryl. The present embodiment provides variations of the compounds set forth in U.S. Pat. No. 20120108815, the entire disclosure of which is hereby incorporated by reference.

In a variation, the compound having formula I is described by formula IA:

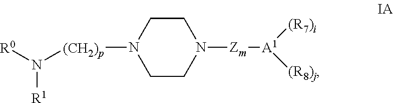

or a pharmaceutically acceptable salt or ester thereof,
wherein:

R⁰ is an optionally substituted C₁₋₈ alkyl, C₁₋₈ alkoxyl, C₂₋₈ alkenyl, C₂₋₈ alkynyl, C₄-8 cycloalkyl, C₄₋₈ cycloalkenyl, C₆₋₁₀ aryl,

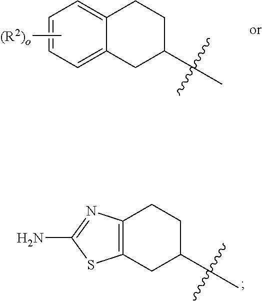

R¹ is an optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, or C₆₋₁₀ aryl;

R² are hydroxyl, C₁₋₄ alkyl, C₁₋₄ alkoxyl, C₁₋₈ alkyl, C₂₋₈ alkenyl, C₂₋₈ alkynyl, C₄₋₈ cycloalkyl, C₄₋₈ cycloalkenyl; C₆₋₁₀ aryl, —NR³_{q} or C₁₋₁₀ hydrocarbon groups optionally containing one or more O, N, S, or Se heteroatoms where R³ individually are H or organyl groups and q is 2 or 3, with the proviso that when q is 3, the group bears a positive formal charge;

R⁷, R⁸ are each independently, H, hydroxyl, oxo, C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo, C₁₋₄ aldehyde, or —NR⁴_{q} where R⁴ is H, C₁₋₈ alkyl, C₂₋₈ alkenyl, C₄₋₈ cycloalkyl, C₄₋₈ cycloalkenyl, 3H-1,2-dithiole-3-thione or C₆₋₁₀ aryl;

A¹ is a C₆₋₁₂ aryl group, C₅₋₁₂ heteroaryl group, substituted 3-hydroxypyridin-4(1H)-one,

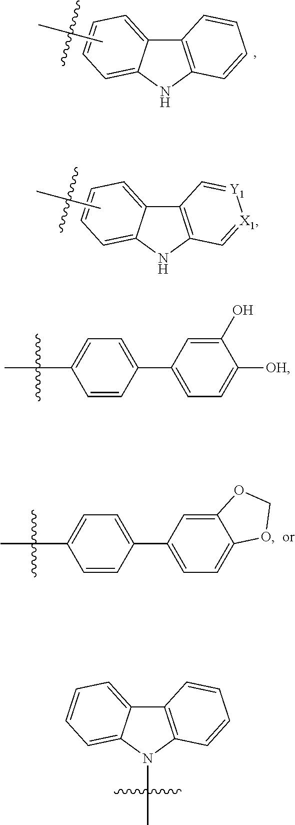

with i hydrogen atoms replaced with R⁷ and j hydrogen atoms replaced with R⁸;

p is an integer from 1 to 6;

X¹, Y¹ are each independently CH or N;

Zₘ is absent or a divalent linking moiety in which Z is repeated m times.

m is an integer from 0 to 5; i, j are each independently 0, 1, 2, or 3; and

o is 0, 1, 2, 3, or 4.

In some refinements, A¹ is:

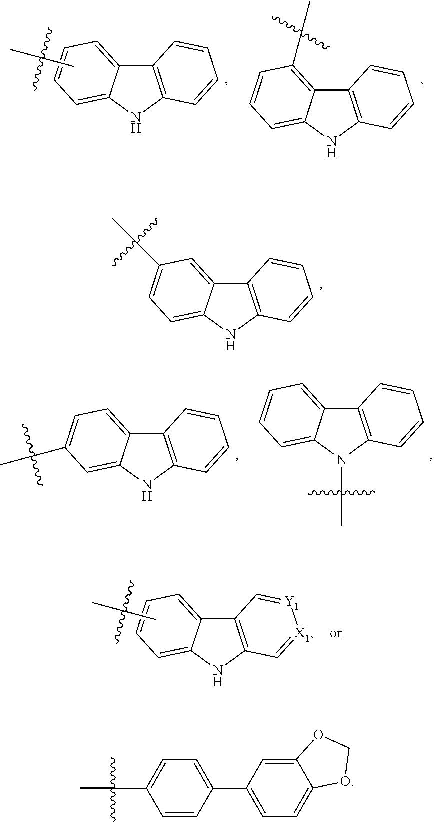

It should be appreciated that each aromatic ring of A¹ can be substituted with R⁷ and/or R⁸ groups. For example,

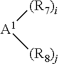

hereafter “A¹(R)ᵢ(R₈)ⱼ” can be described by:

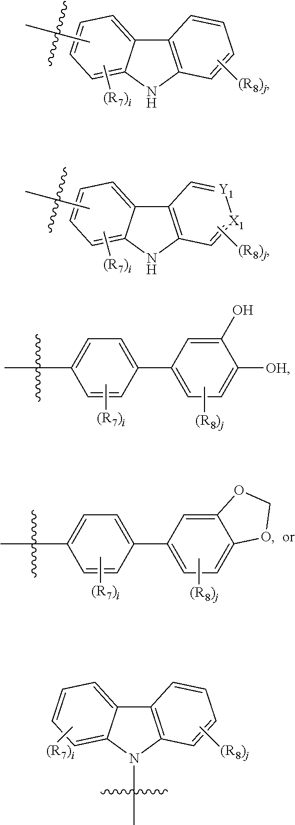

In further refinements, R¹ is substituted with a component selected from the group consisting of C₁₋₈ alkoxy, C₁₋₄ acyloxy, C₁₋₄ acyl, —C(O)—R⁴, and —R⁵—NH—SO₂—NR⁴ᵣ where R⁵—NH—C(O)—R⁴; —R⁵—NR⁴ᵣ, or —R⁵—Ar where R⁴ is H, C₁₋₈ alkyl, C₂₋₈ alkenyl, C₄₋₈ cycloalkyl, C₄₋₈ cycloalkenyl, or C₆₋₁₀ aryl; R⁵ is C₂₋₈ alkenyl; r is 2 or 3; and Ar is a C₆₋₁₀ aryl ring system, optionally including one or more heteroatoms or C₅₋₁₀ heteroaryl; with the proviso that when r is 3, the nitrogen of the NR⁴ᵣ group will bear a positive formal charge. In a refinement, Ar is an optionally substituted phenyl, thienyl, pyridyl, bipyridyl, biphenylyl, or naphthyl.

In further refinements, R² are hydroxyl, C₁₋₈ alkyl, C₁₋₈ alkoxyl, C₂₋₈ alkenyl, C₂₋₈ alkynyl, C₄₋₈ cycloalkyl, C₄₋₈ cycloalkenyl; C₆₋₁₀ aryl, —NR³_{q} —NH—C(O)—R³, or —NH—C(O)—NR⁴₂, where R³ individually are H, C₁₋₈ alkyl, C₂₋₈ alkenyl, C₂₋₁₀ alkynyl, C₄₋₈ cycloalkyl, C₄₋₈ cycloalkenyl, or C₆₋₁₀ aryl where q is 2 or 3, with the proviso that when q is 3, the group bears a positive formal charge and wherein the hydrocarbon groups in each case are optionally substituted with —CN, C₁₋₈ alkyl, —OR³, —OH, halo, or —CF₃.

In some variations, A or A¹ are a C₆₋₁₂ aryl group consisting of 1 to 4 rings or a C₅₋₁₂ heteroaryl optionally linked, and optionally substituted by C₁₋₄ alkyl, C₂₋₄ alkenyl, C₂₋₁₀ alkynyl, C₅₋₇ cycloalkyl, C₅₋₇ cycloalkenyl, halo, C₁₋₄ aldehyde, or —NR⁴_{q}, where R⁴ is C₁₋₈ alkyl, OH, C₁₋₈ alkoxyl, C₂₋₈ alkenyl, C₄₋₈ cycloalkyl, C₄₋₈ cycloalkenyl, or C₆₋₁₀ aryl; and q is 2 or 3, with the proviso that when q is 3, the group bears a positive formal charge. In a refinement, A is an optionally substituted thienyl, pyridinyl, phenyl, biphenyl, naphthyl, quinolone, indole, or isoquinoline. For example, A can be substituted with C₁₋₈ alkyl, —CN, halo, OH, C₁₋₈ alkoxyl, NH₂SO₂R³, CF₃, arylsulfonyl, arylsulfonamide, o-OCH₃, 2,3-dichloro, pyridinyl, bipyridinyl, or p-NHSO₂CH₃.

In other refinements, R¹, R², R⁷, R⁸, Z, Zₘ, m, o, p, X, Y, and A for formula IA are the same as set forth above for formula I.

A variation of the compounds having formula I are described by formula IB and IC:

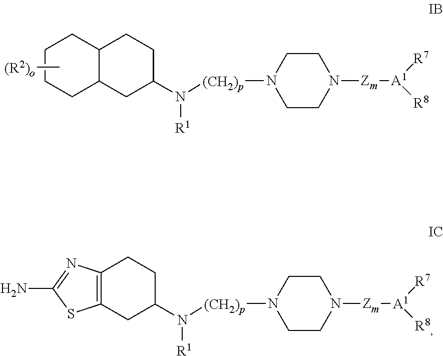

where R¹, R², R⁷, R⁸, Z, Zₘ, m, o, p, X, Y, and A are the same as set forth above for formula I and IA.

In another embodiment, a compound having formula II for treating neurodegenerative and other related CNS diseases is provided:

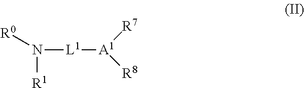

where R⁰, R¹, R², R⁷, R⁸, o, p, i, j, X, Y, and A¹ are the same as set forth above. L¹ is a linking group. In a refinement, L¹ is (CH₂)ₙ, (CH₂)ₙCO, (CH₂)ₙNCO(CH₂)ₖ, C₁₋₁₀ carboximido, C₁₋₁₀ alkyl (i.e., an C₁₋₁₀ alkanediyl), C₂₋₁₀ alkenyl (i.e., an C₂₋₁₀ alkanediyl), (CH₂)ₙCH═CH(CH₂)ₖ, (CH₂)ₙCC(CH₂)ₖ, C₂₋₁₀ alkynyl (i.e., an C₂₋₁₀ alkynediyl), where n and k are integers from 0 to 8; and R⁷, R⁸ each independently, hydrogen (H), hydroxyl, C₁₋₈ alkyl, C₂₋₈ alkenyl, C₂₋₁₀ alkynyl, C₅₋₇ cycloalkyl, C₅₋₇ cycloalkenyl, halo, preferably fluoro or chloro, C₁₋₄ aldehyde, —NR⁴_{q} and like groups where R⁴ is H or organyl groups, preferably H, C₁₋₈ alkyl, C₂₋₈ alkenyl, C₂₋₁₀ alkynyl, C₄₋₈ cycloalkyl, C₄₋₈ cycloalkenyl, or C₆₋₁₀ aryl. In a refinement, n and k are each independently, 0, 1, 2, 3, 4, 5, 6, 7, and 8.

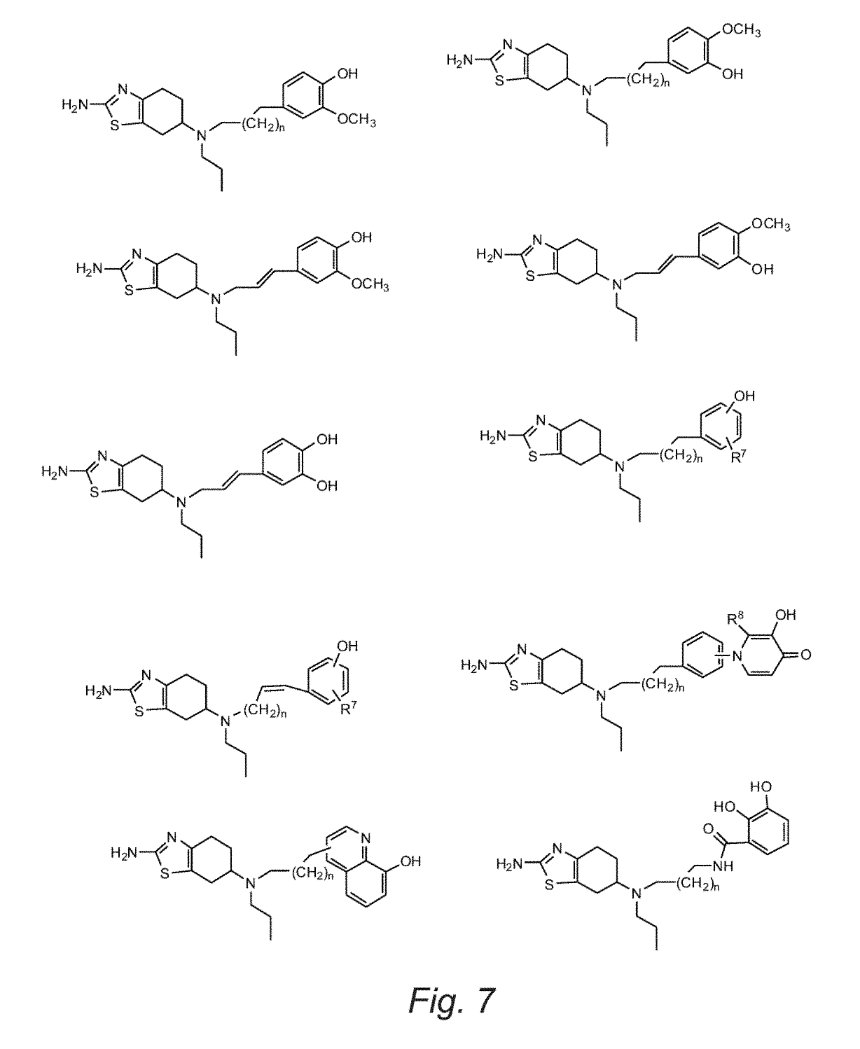

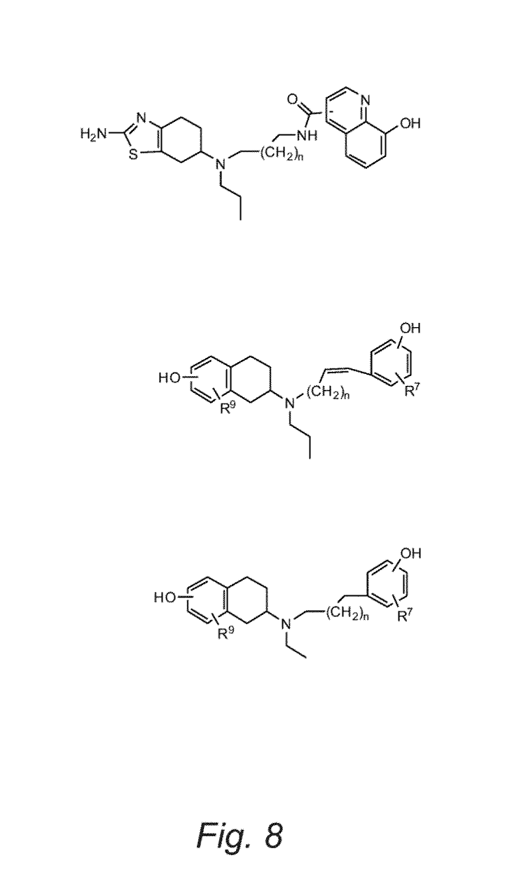

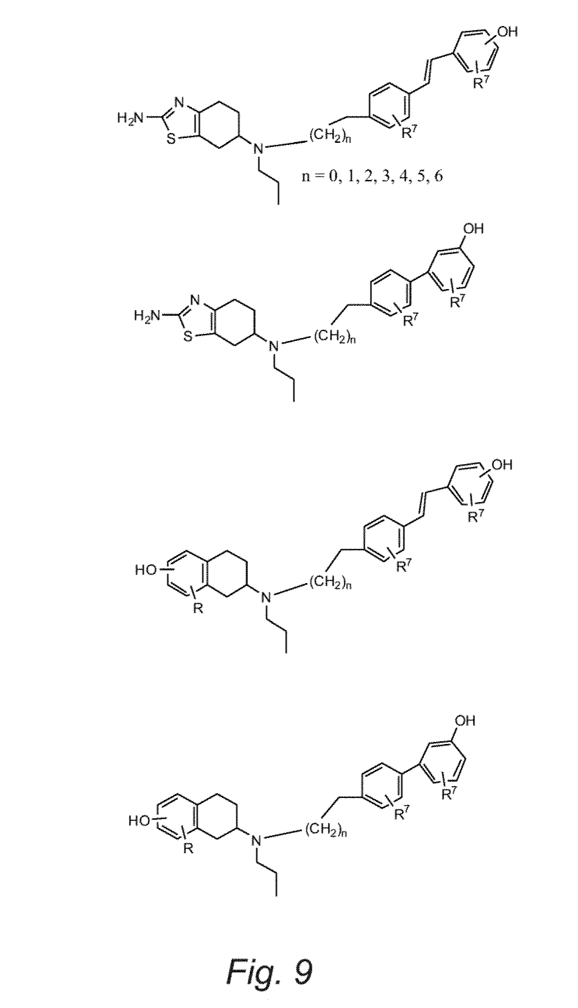

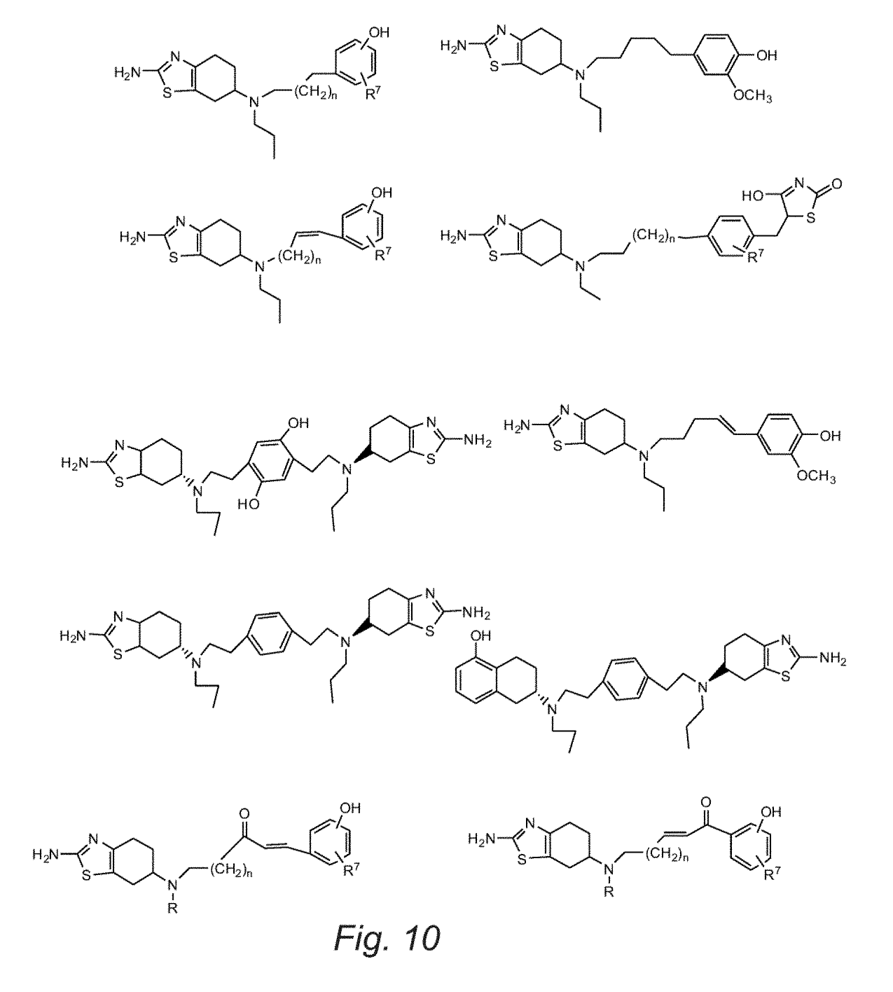

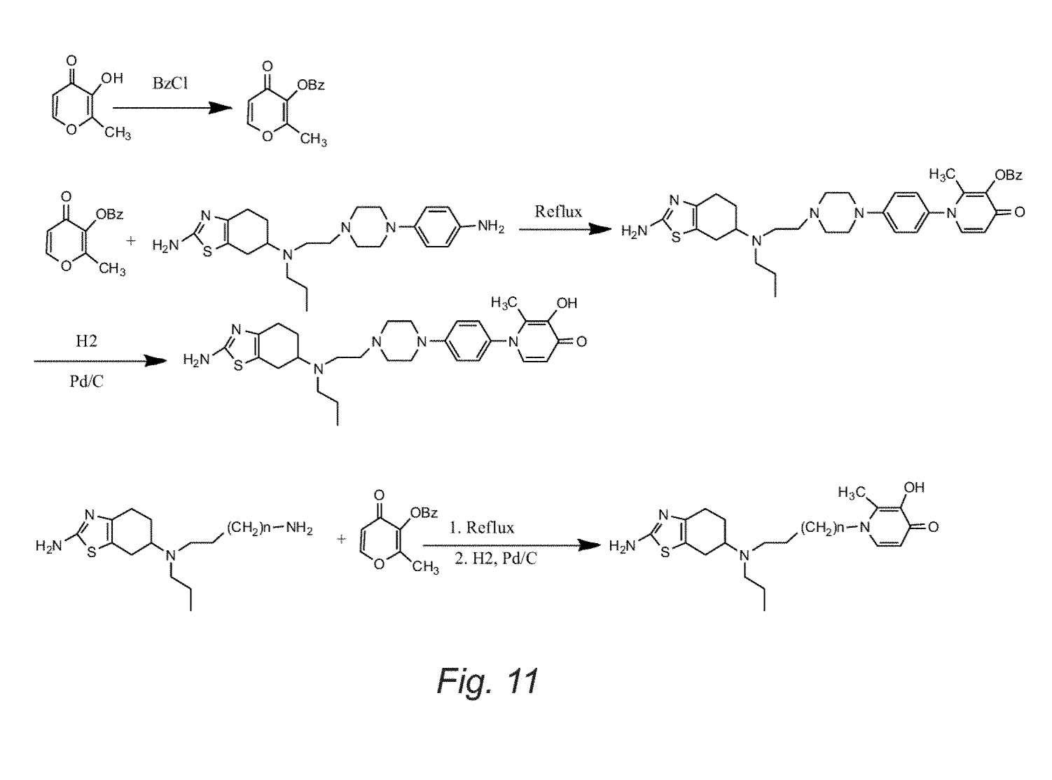

FIGS. 7-10 provide examples of the compounds having formula II while FIG. 11 provides a synthetic scheme. In these Figures, n is 0 to 8 and more particularly, n is 0, 1, 2, 3, 4, 5, or 6. R⁷, R⁸ are as set forth above. In particular, R⁷, R⁸, R⁹ is hydrogen (H), hydroxyl, C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo, preferably fluoro or chloro, C₁₋₄ aldehyde, —NR⁴_{q} and like groups where R⁴ is H or organyl groups, preferably H, C₁₋₈ alkyl, C₂₋₈ alkenyl, C₂₋₁₀ alkynyl, C₄₋₈ cycloalkyl, C₄₋₈ cycloalkenyl, or C₆₋₁₀ aryl.

A variation of the compounds having formula II are described by formula IIA and IIB:

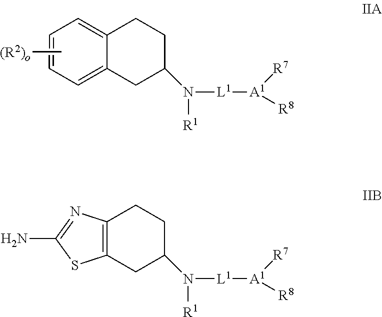

where R¹, R², R⁷, R⁸, L₁, o, and A are the same as set forth above.

In a refinement of the compounds having formula I, IA, IB, IC, II, IIA, and IIB, A or A¹ is selected from the group consisting of:

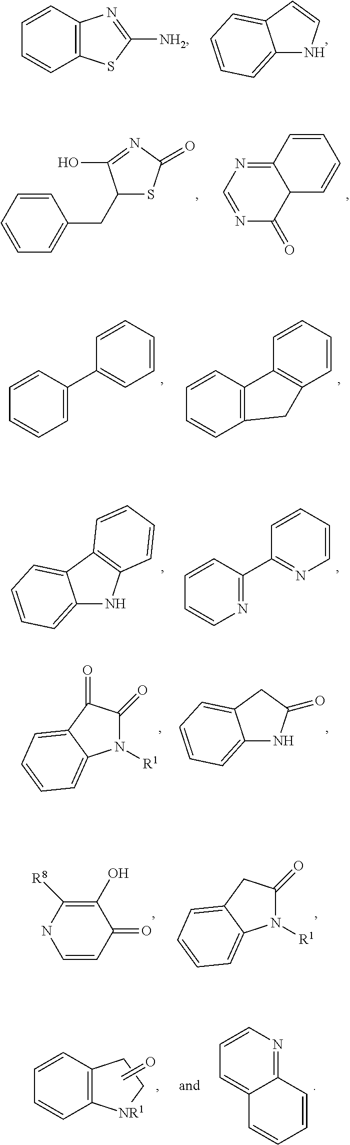

The compounds set forth herein may be used per se or as pharmaceutically acceptable derivatives. The latter term includes salts, esters, and other derivatives generally considered acceptable by pharmaceutical standards. Useful derivatives, for example, include salts of organic and inorganic acids such as sulfates, phosphates, hydrohalide salts, carboxylate salts, etc., as well as esters of carboxylic acid or hydroxyl substituents, ethers of hydroxyl substituents, amides of amino substituents, as well as carbamates, ureas, etc. Synthesis of these derivatives is conventional, and well known to those skilled in pharmaceutical chemistry. For example, compounds bearing hydroxyl groups may be converted to esters by customary techniques of organic chemistry, such as reaction with an acyl halide, carboxylic acid anhydride, or by esterification with an acid while removing byproduct water. In some cases, derivation may be desired to facilitate compounding of the pharmaceutical into an acceptable form such as tablets, powder, aqueous dispersion, capsule, etc., or may be useful in assisting bioavailability of the drug following administration, for example, by rendering the compound more or less soluble. In many cases, such as, for example, esters, ureas, carbamates, ethers, etc., the derivative may act as “prodrug,” which liberates the active form by biological transformation, i.e., by enzymatic hydrolysis of an ester functionality, as is well known to the pharmaceutical chemist.

Typical dosages for mammalian species may vary from 0.001 mg/Kg of body weight to about 100 mg/Kg of body weight, preferably 0.01 mg/Kg to 5 mg/Kg. The actual amount will vary depending upon the particular CNS activity desired to be altered, and the desired degree of alteration. The upper limits may, as with virtually all drugs, be limited by toxicity of the drug or its metabolites, or by the presence of unwanted side effects. The drugs may be administered in any form, but preferably in the form of tablets or capsules with appropriate excipients. Dosages, forms of administration, etc., can be readily determined by those skilled in the art.

Guidelines to the effective dosages in mammalian species are provided by the many known drugs commercially available which bind to CNS monoamine receptor sites, and by comparing the binding affinities of these pharmaceuticals with the target compounds of the subject invention by in vivo and in vitro studies. In addition to the utility of the subject invention compounds in treatment of diseases such as Parkinson's disease, schizophrenia, treatment for addiction such as cocaine addiction, and the like, the subject invention compounds are also useful, particularly in their radio labeled form, for clinical studies directed to distribution of monoamine receptor sites in the brain and the effect which compounds, such as cocaine, have on these sites.

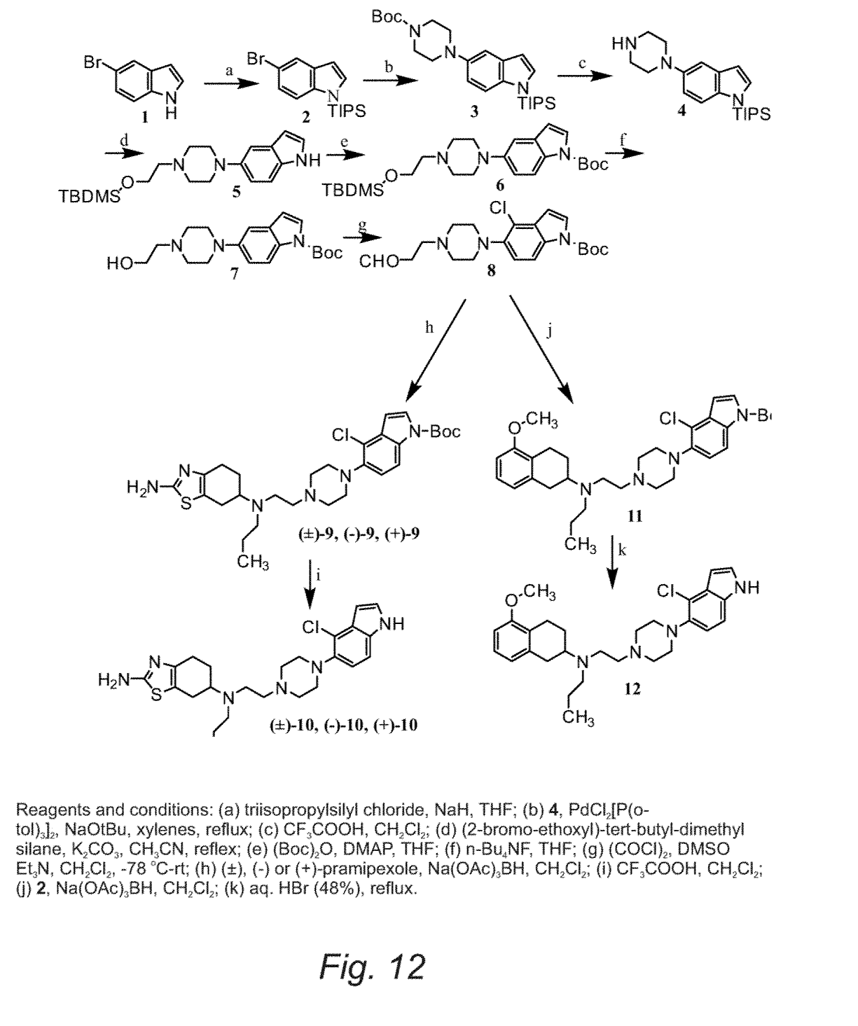

FIG. 12 provides a synthetic scheme for making compounds having formula I. The reaction conditions for this scheme are: (a) trisisopropylsilyl chloride, NaH, THF; (b) 4, PdCl₂(P(o-tol)₃]₂, NaOtBu, xylenes, reflux; (c) CF₃COOH, CH₂Cl₂, (d) (2-bromo-ethoxy)-tert-butyl-dimethyl silane, K₂CO₃, CH₃CN, reflux; (e) (Boc)₂O, DMAP, THF; (f) n-Bu₄NF, THF; (g) (COCl)₂, DMSO, Et₃N, CH₂Cl₂, −78° C.-rt; (h)(+/−), (−) or (+)-pramipexole, Na(OAc)3BH, CH₂Cl₂; (i) CF₃COOH, CH₂Cl₂; (j) 2, Na(OAc)3BH, CH₂Cl₂; (k) aq. HBr (48%), reflux.

The following examples illustrate the various embodiments of the present invention. Those skilled in the art will recognize many variations that are within the spirit of the present invention and scope of the claims.

### 5-Bromo-1-(triisopropylsilyl)-1H-indole (2)

Into a stirring solution of NaH (4.03 g, 170.0 mmol) in dry THF (150 mL), compound 1 (16.44 g, 83.9 mmol) was added portion wise at 0° C. The reaction mixture was allowed to stir at room temperature for 1 h, followed by dropwise addition of triisopropylsilyl chloride (20 g, 103.7 mmol). The reaction mixture was stirred for 12 h and then filtered through celite. The crude residue was purified by column chromatography using hexane as solvent to afford compound 2 (22 g, 75%). ¹H NMR (CDCl₃, 400 MHz): δ 1.23 (s, 18H), 1.74 (heptet, J=7.6 Hz, 3H), 6.64 (d, J=3.2 Hz, 1H), 7.07 (d, J=6 Hz, 1H), 7.14 (s, 1H), 7.31 (d, J=3.2 Hz, 1H), 7.45 (d, J=8.8, 1H).

### tert-Butyl 4-(1-(triisopropylsilyl)-1H-indol-5-yl)piperazine-1-carboxylate (3)

A mixture of compound 2 (22.0 g, 63.0 mmol), tert-butyl piperazine-1-carboxylate (11.71 g, 63.0 mmol), PdCl₂[P(O-tol)₃]₂(2.47 g, 3.1 mmol) and NaOtBu (9.08 g, 94.4 mmol) in xylenes (175 mL) was heated at 110° C. for 12 h. The reaction mixture was filtered through celite and concentrated in vacuo. The crude residue was purified by column chromatography (EtOAc/hexane, 1:20) to afford compound 3 (13.22 g, 46%). ¹H NMR (CDCl₃, 400 MHz): δ 1.19 (s, 18H), 1.55 (s, 9H), 1.74 (heptet, J=6.8 Hz, 3H), 3.14 (bs, 4H), 3.67 (bs, 4H), 6.60 (t, J=6 Hz, 1H), 6.94 (d, J=8.8, 1H), 7.19 (s, 1H), 7.26 (t, J=2.8 Hz, 1H), 7.47 (d, J=8.8 Hz, 1H).

### Procedure C. 5-Piperazin-1-yl-1H-indole (4)

To a stirring solution of compound 3 (7.70 g, 16.8 mmol) in CH₂Cl₂ (15 mL), TFA (15 mL) was added slowly at room temperature and the reaction mixture was stirred for 2 h. Unreacted TFA and solvent were removed under reduced pressure and the salt was washed with diethylether. A saturated solution of NaHCO₃ was added to the salt, followed by extraction with CH₂Cl₂ (3×50 mL). The combined organic layer was dried over Na₂SO₄, filtered and evaporated in vacuo to provide compound 4 (2.88 g, 85%). ¹H NMR (CDCl₃, 400 MHz): δ 1.85 (bs, 1H), 2.80-3.28 (m, 8H), 6.85-7.10 (m, 1H), 7.02-7.40 (m, 4H), 8.31 (bs, 1H).

### Procedure D. 5-{4-[2-(tert-Butyl-dimethyl-silanyloxy)ethyl]piperazin-1-yl}-1H-indole (5)

A mixture of compound 4 (2.88 g, 14.3 mmol), (2-bromoethoxy)-tert-butyl-dimethylsilane (3.42 g, 14.3 mmol) and K₂CO₃ (5.93 g, 42.9 mmol) in CH₃CN (50 mL) was refluxed for 14 hours. After filtration, acetonitrile was evaporated under reduced pressure and the crude material was purified by silica gel column chromatography (EtOAc/hexane, 3:1) to give compound 5 (4.01 g, 78%). ¹H NMR (CDCl₃, 400 MHz): δ 0.02 (s, 6H), 0.83 (s, 9H), 2.30-2.80 (m, 6H), 2.82-3.30 (m, 4H), 3.52-3.82 (m, 2H), 6.25-6.48 (m, 1H), 6.75-7.30 (m, 4H), 8.09 (s, 1H).

### 5-{4-[2-(tert-Butyl-dimethyl-silanyloxy)ethyl]piperazin-1-yl}indole-1-carboxylic Acid tert-butyl Ester (6)

Amine 5 (4.0 g, 11.1 mmol) was reacted with (Boc)₂O (2.68 g, 12.2 mmol) and DMAP (1.49 g, 12.2 mmol) in THF (50 mL) at room temperature using procedure G. The crude material was purified by column chromatography over silica gel (EtOAc/hexane, 1:1) to give compound 6 (5.2 g, ˜100%). ¹H NMR (CDCl₃, 400 MHz): δ 0.08 (s, 6H), 0.95 (s, 9H), 1.65 (s, 9H), 2.61 (t, J=6.4 Hz, 2H), 2.73 (t, J=4.8 Hz, 4H), 3.19 (t, J=4.8 Hz, 4H), 3.81 (t, J=6.4 Hz, 2H), 6.47 (d, J=3.6 Hz, 1H), 7.01 (dd, J=6.4, 2.4 Hz, 1H), 7.06 (dd, J=6.4, 2.4 Hz, 1H), 7.53 (s, 1H), 8.00 (s, 1H).

### Procedure E. 5-[4-(2-Hydroxy-ethyl)piperazin-1-yl]indole-1-carboxylic Acid tert-butyl Ester (7)

Into a stirring solution of compound 6 (2.0 g, 4.3 mmol) in THF (30 mL), n-tetrabutylammonium fluoride (1.14 g, 4.3 mmol, 1.0 M solution in THF) was added at 0° C. The reaction mixture was then stirred at room temperature for 1 h. THF was evaporated in vacuo, the residue was diluted with CH₂Cl₂ (50 mL) and washed with water. The water layer was extracted with CH₂Cl₂ (3×75 mL). The combined organic layer was washed with brine, dried over Na₂SO₄, and evaporated. The crude product was purified by silica gel column chromatography (EtOAc/MeOH, 20:1) to yield compound 7 (1.49 g, 99%). ¹H NMR (CDCl₃, 400 MHz): δ 1.65 (s, 9H), 2.61 (t, J=5.2 Hz, 2H), 2.70 (t, J=4.8 Hz, 4H), 3.19 (t, J=4.8 Hz, 4H), 3.67 (t, J=5.2 Hz, 2H), 6.47 (d, J=3.6 Hz, 1H), 7.01 (dd, J=6.8, 2 Hz, 1H), 7.06 (d, J=2 Hz, 1H), 7.53 (s, 1H), 8.00 (s, 1H).

### tert-butyl 4-chloro-5-(4-(2-oxoethyl)piperazin-1-yl)-1H-indole-1-carboxylate (8)

Compound 7 (1.49 g, 4.3 mmol) was reacted with oxalyl chloride (0.75 mL, 8.6 mmol), DMSO (1.23 mL, 17.3 mmol) and Et₃N (3.6 mL, 25.8 mmol) in CH₂Cl₂ (40 mL) using procedure A. The crude residue was purified by column chromatography using ethyl acetate as solvent to afford compound 8 (1.23 g, 83%).

### tert-butyl 5-(4-(2-((2-amino-4,5,6,7-tetrahydrobenzo[d]thiazol-6-yl)(propyl)amino)ethyl)piperazin-1-yl)-4-chloro-1H-indole-1-carboxylate [(+)-9]

Compound 8 (175 mg, 0.51 mmol) was reacted with (±)-pramipexole (108 mg, 0.51 mmol) and NaBH(OAc)₃ (194 mg, 0.92 mmol) in CH₂Cl₂ (15 mL) according to procedure B. The crude product was purified by silica gel column chromatography (EtOAc/MeOH, 20:1) to yield compound (+)-9 (150 mg, 55%). ¹H NMR (CDCl₃, 400 MHz): δ 0.91 (t, J=6.8 Hz, 1H), 1.35-1.60 (m, 2H), 1.67 (s, 9H), 1.89-2.10 (m, 1H), 2.30-3.30 (m, 20H), 4.94 (bs, 2H), 6.67 (t, J=3.2 Hz, 1H), 7.09 (dd, J=8.8, 2.8 Hz, 1H), 7.28 (dd, J=3.2 Hz, 1H), 7.59 (s, 1H), 7.97 (d, J=6.4 Hz, 1H).

### (S)-tert-butyl 5-(4-(2-((2-amino-4,5,6,7-tetrahydrobenzo[d]thiazol-6-yl)(propyl)amino)ethyl)piperazin-1-yl)-4-chloro-1H-indole-1-carboxylate [(−)-9]

Compound 8 (175 mg, 0.51 mmol) was reacted with S-(−)-pramipexole (108 mg, 0.51 mmol) and NaBH(OAc)₃ (194 mg, 0.92 mmol) in CH₂Cl₂ (15 mL) using procedure B. The crude residue was purified by column chromatography (EtOAc/MeOH, 20:1) to afford compound S-(−)-9 (161 mg, 59%). ¹H NMR (CDCl₃, 400 MHz): δ 0.93 (t, J=6.8 Hz, 1H), 1.35-1.60 (m, 2H), 1.68 (s, 9H), 1.89-2.10 (m, 1H), 2.30-3.30 (m, 20H), 4.94 (bs, 2H), 6.67 (t, J=3.2 Hz, 1H), 7.09 (dd, J=8.8, 2.8 Hz, 1H), 7.28 (dd, J=3.2 Hz, 1H), 7.60 (s, 1H), 7.97 (d, J=6.4 Hz, 1H).

### (R)-tert-butyl 5-(4-(2-((2-amino-4,5,6,7-tetrahydrobenzo[d]thiazol-6-yl)(propyl)amino)ethyl)piperazin-1-yl)-4-chloro-1H-indole-1-carboxylate [(+)-9]

Compound 8 (175 mg, 0.51 mmol) was reacted with R-(+)-pramipexole (108 mg, 0.51 mmol) and NaBH(OAc)₃ (194 mg, 0.92 mmol) in CH₂Cl₂ (15 mL) using procedure B. The crude residue was purified by column chromatography using (EtOAc/MeOH, 20:1) to afford compound R-(+)-9 (164 mg, 60%). ¹H NMR (CDCl₃, 400 MHz): δ 0.91 (t, J=6.8 Hz, 1H), 1.37-1.60 (m, 2H), 1.67 (s, 9H), 1.89-2.10 (m, 1H), 2.30-3.30 (m, 20H), 4.94 (bs, 2H), 6.67 (t, J=3.2 Hz, 1H), 7.10 (dd, J=8.8, 2.8 Hz, 1H), 7.28 (dd, J=3.2 Hz, 1H), 7.59 (s, 1H), 7.98 (d, J=6.4 Hz, 1H).

### N⁶-(2-(4-(4-chloro-1H-indol-5-yl)piperazin-1-yl)ethyl)-N⁶-propyl-4,5,6,7-tetrahydrobenzo[d]thiazole-2,6-diamine [(±)-10]

Compound (+)-9 (150 mg, 0.28 mmol) was reacted with TFA (10 mL) in CH₂Cl₂ (10 mL) using procedure C. Unreacted TFA and solvent were removed in vacuo and the salt was washed with diethylether and recrystallized from ethanol to afford compound (±)-10 (106 mg, 38%). ¹H NMR (CD₃OD, 400 MHz): δ 0.99 (t, J=7.2 Hz, 3H), 1.52-1.74 (m, 2H), 1.76-2.04 (m, 1H), 2.19 (d, J=9.2 Hz, 1H), 2.52-2.84 (m, 6H), 3.10-3.58 (m, 13H), 6.50 (d, J=3.2 Hz, 1H), 7.05 (d, J=8.8 Hz, 1H), 7.29 (d, J=3.2 Hz, 1H), 7.34 (d, J=8.8 Hz, 2H). ¹³C (CD₃OD, 100 MHz): δ 12.0, 22.2, 24.0, 25.1, 47.0, 51.1, 54.4, 54.8, 59.2, 101.4, 101.5, 111.8, 115.3, 116.1, 127.8, 129.7, 135.1, 136.0, 140.7, 171.0. M.p. 110-115° C. Anal. calculated for C₃₀H₄₀F₉N₆O_{7.5}S: C, H, N.

### (S)—N⁶-(2-(4-(4-chloro-1H-indol-5-yl)piperazin-1-yl)ethyl)-N⁶-propyl-4,5,6,7-tetrahydrobenzo[d]thiazole-2,6-diamine [(−)-10]

Compound (−)-9 (150 mg, 0.28 mmol) was reacted with TFA (10 mL) in CH₂Cl₂ (10 mL) using procedure C. Unreacted TFA and solvent were removed in vacuo and the salt was washed with diethylether and recrystallized from ethanol to afford compound S-(−)-10 (120 mg, 43%). ¹H NMR (CD₃OD, 400 MHz): δ 0.98 (t, J=7.2 Hz, 3H), 1.54-1.74 (m, 2H), 1.76-2.04 (m, 1H), 2.19 (d, J=9.2 Hz, 1H), 2.52-2.84 (m, 6H), 3.10-3.58 (m, 13H), 6.51 (d, J=3.2 Hz, 1H), 7.05 (d, J=8.8 Hz, 1H), 7.29 (d, J=3.2 Hz, 1H), 7.34 (d, J=8.8 Hz, 2H). ¹³C (CD₃OD, 100 MHz): δ 12.0, 22.3, 24.0, 25.1, 47.0, 51.1, 54.5, 54.8, 59.2, 101.4, 101.6, 111.8, 115.3, 116.1, 127.8, 129.7, 135.1, 136.0, 140.8, 171.0. [α]²⁵_{D}=−11.0° (c=1.0, CH₃OH). M.p. 115-120° C. Anal. calculated for C₃₁H_{37.5}F_{10.5}N₆O₇S: C, H, N.

### (R)—N⁶-(2-(4-(4-chloro-1H-indol-5-yl)piperazin-1-yl)ethyl)-N⁶-propyl-4,5,6,7-tetrahydrobenzo[d]thiazole-2,6-diamine [(+)-10]

Compound (+)-9 (150 mg, 0.28 mmol) was reacted with TFA (10 mL) in CH₂Cl₂ (10 mL) using procedure C. Unreacted TFA and solvent were removed in vacuo and the salt was washed with diethylether and recrystallized from ethanol to afford compound R-(+)-10 (140 mg, 50%). ¹H NMR (CD₃OD, 400 MHz): δ 0.98 (t, J=7.2 Hz, 3H), 1.54-1.78 (m, 2H), 1.76-2.04 (m, 1H), 2.20 (d, J=9.2 Hz, 1H), 2.52-2.84 (m, 6H), 3.10-3.58 (m, 13H), 6.51 (d, J=3.2 Hz, 1H), 7.05 (d, J=8.8 Hz, 1H), 7.29 (d, J=3.2 Hz, 1H), 7.34 (d, J=8.8 Hz, 2H). ¹³C (CD₃OD, 100 MHz): δ 12.2, 22.3, 24.0, 25.1, 47.0, 51.2, 54.5, 54.8, 59.2, 101.4, 101.6, 111.8, 115.3, 116.1, 127.8, 129.7, 135.1, 136.2, 140.8, 171.0. [α]²⁵_{D}=−15.5° (c=1.0, CH₃OH). M.p. 115-120° C. Anal. calculated for C₃₁H_{37.9}F_{10.5}N₆O_{7.2}S: C, H, N.

### tert-butyl 4-chloro-5-(4-(2-((5-methoxy-1,2,3,4-tetrahydronaphthalen-2-yl)(propyl)amino)ethyl)piperazin-1-yl)-1H-indole-1-carboxylate (11)

Aldehyde 8 (320 mg, 0.93 mmol) was reacted with 5-methoxy-N-propyl-1,2,3,4-tetrahydronaphthalen-2-amine (204 mg, 0.93 mmol) and NaBH(OAc)₃ (355 mg, 1.68 mmol) in CH₂Cl₂ (20 mL) using procedure B. The crude material was purified by column chromatography over silica gel (EtOAc/hexane, 1:1) to give compound 11 (190 mg, 38%). ¹H NMR (CDCl₃, 400 MHz): δ 0.91 (t, J=7.2 Hz, 3H), 1.33-1.75 (m, 13H), 1.95-2.13 (m, 1H), 2.35-3.23 (m, 18H), 3.81 (s, 3H), 6.55-6.77 (m, 3H), 7.03-7.15 (m, 3H), 7.58 (d, J=2.4 Hz, 1H), 7.96 (d, J=6.8 Hz, 1H).

### 6-((2-(4-(4-chloro-1H-indol-5-yl)piperazin-1-yl)ethyl)(propyl)amino)-5,6,7,8-tetrahydronaphthalen-1-ol (12)

A mixture of compound 11 (60 mg, 0.11 mmol) and 48% aq. HBr (10 ml) was refluxed for 5 h. The reaction mixture was evaporated to dryness and the residue was washed with diethylether. Finally, the HBr salt was recrystallized from ethanol to furnish compound 12 (50 mg, 60%). ¹H NMR (CD₃OD, 400 MHz): δ 0.95 (t, J=7.2 Hz, 3H), 1.41-1.57 (m, 3H), 2.00-2.22 (m, 1H), 2.58-3.18 (m, 19H), 6.61 (d, J=8 Hz, 1H), 6.75 (d, J=8 Hz, 1H), 7.08 (t, J=7.6 Hz, 1H), 7.19-7.38 (m, 3H), 7.51 (d, J=8 Hz, 1H), 7.90 (d, J=8.2 Hz, 1H), 8.50 (bs, 1H). M.p. 250-260° C. Anal. calculated for C₂₇H_{41.4}Br₄N₄O_{1.7}: C, H, N.

TABLE 1
Inhibition constants for competition with [³H]spiroperidol binding
to cloned rat D2L and D3 receptors expressed in HEK-293 cells.

| | Kᵢ (nM), rD2L | Kᵢ (nM), rD3 | |
|---|---|---|---|
| Compound | [³H]spiroperidol | [³H]spiroperidol | D2L/D3 |
| (±)-10 | 46.7 ± 6.6 | 1.92 ± 0.38 | 24.3 |
| (−)-10 | 39 ± 5 | 2.19 ± 0.39 | 17.8 |
| (+)-10 | 134 ± 12 | 15.9 ± 3.6 | 8.46 |
| 12 | 76.4 ± 2.4 | 10.4 ± 1.6 | 7.3 |

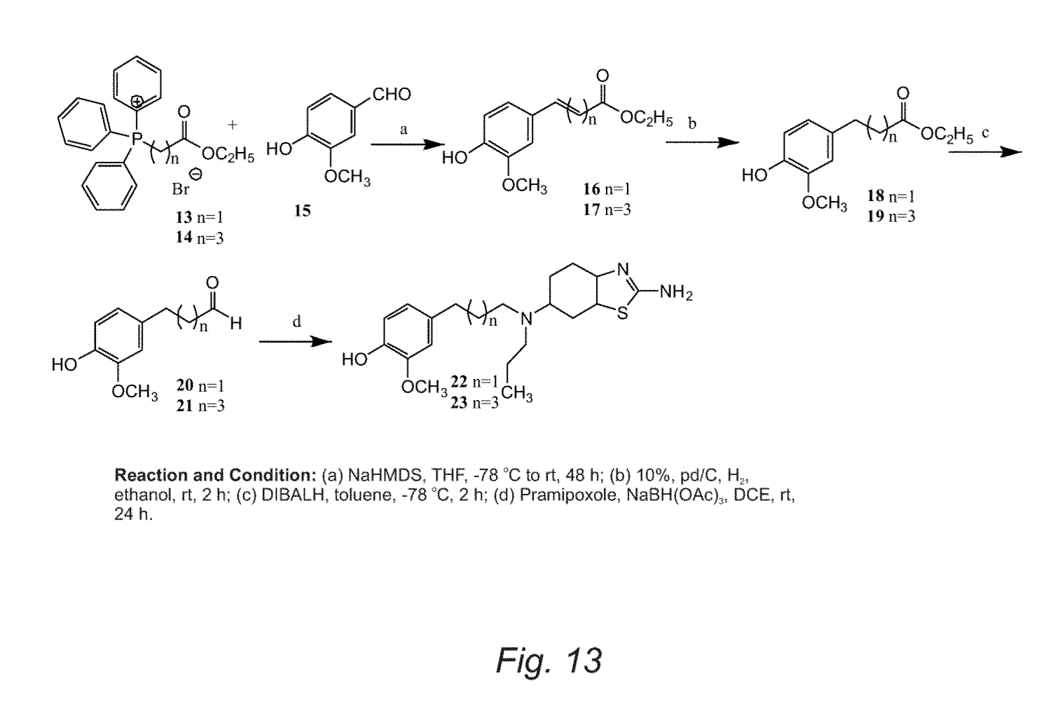

FIG. 13 provides a synthetic scheme for the compounds having formula II. The reaction conditions for this scheme are: (a) NaHMDS, THF, −78° C. to rt, 48 h; (b) 10% pd/C, H₂, ethanol, rt, 2 h; (c) DIBALH, toluene, −78° C., 2 h; (d) Pramipexole, NaBH(OAc)₃, DCE, rt, 24 h.

### Procedure A

### Ethyl 3-(4-hydroxy-3-methoxyphenyl)acrylate (16) and Ethyl 5-(4-hydroxy-3-methoxyphenyl)pent-4-enoate (17)

Commercially available (ethoxycarbonylmethyl)triphenylphosphonium bromide 13 (3.10 g, 7.23 mmol) was added to dry THF (8 mL) in an oven-dried round bottom flask. The resulting suspension was cooled to −78° C. Then a solution of NaHMDS (1M in THF, 7.88 mL, 7.88 mmol) was added drop-wise and the reaction mixture stirred at −78° C. for 1 h. Thereafter, a solution of Vanillin 15 (1.0 g, 6.57 mmol) in dry THF (2 mL) was added drop-wise and reaction stirred at −78° C. for 2 h, then warmed to room temperature and stirred for 48 h. The reaction mixture was then extracted with ethyl acetate (2×75 mL) and washed with brine. The combined organic layer was dried over sodium sulfate and concentrated in vacuo. The crude thus obtained was purified by column chromatography (Hexane/ethyl acetate 9:1) to yield compound 16 (1.25 g, 85%) as colorless oil with a preferential Z:E ratio of >20:1: ¹H NMR (400 MHz, CDCl₃) δ ppm 1.33 (t, 3H, J=7.6 Hz, CH₃), 3.92 (s, 3H, OCH₃), 4.25 (q, 2H, J=7.2 Hz, OCH₂), 5.83 (m, 1H, CH), 6.29 (d, 1H, J=15.2 Hz, CH), 6.91 (d, 1H, J=8.0 Hz, ArH), 7.03 (m, 2H, ArH)

### Preparation of starting material [3-(Ethoxycarbonyl)propyl]triphenylphosphonium Bromide (14)

Triphenylphosphine (3.76 g, 14.35 mmol) and ethyl 4-bromobutyrate (2 g, 10.25 mmol) were added to a dried round bottom flask under argon. The reaction mixture was heated to 120° C. under condenser for 16 h after which the reaction was allowed to come to room temperature. DCM (10 mL) was added, followed by diethyl ether until no further precipitation of product was observed. The precipitate was further washed with ether (100 mL) and solvent dried in vacuo to give pure compound 14 as a white solid (4.64 g) in quantitative yields. ¹H NMR (400 MHz, CDCl₃) δ ppm 1.22 (t, 3H, J=7.2 Hz, CH₃), 1.88-1.94 (m, 2H, CH₂), 2.86-2.89 (m, 2H, CH₂), 3.98-4.12 (m, 4H, CH₂), 7.67-7.90 (15H, ArH).

Starting material 14 (3.30 g, 7.23 mmol) was reacted with vanillin 15 (1.0 g, 6.57 mmol) according to procedure A and the crude product was purified using column chromatography (Hexane/ethyl acetate 9:1) to yield compound 17 (1.17 g, 71%) as colorless oil with a preferential Z:E ratio of >20:1: ¹H NMR (400 MHz, CDCl₃) δ ppm 1.24 (t, 3H, J=7.2 Hz, CH₃), 2.45 (t, 2H, J=8.0 Hz, CH₂), 2.63-2.69 (m, 2H, CH₂), 3.88 (s, 3H, OCH₃), 4.13 (q, 2H, J=7.2 Hz, OCH₂), 5.49-5.55 (m, 1H, CH), 5.64 (s, 1H, OH), 6.37 (d, 1H, J=11.2 Hz, CH), 6.79-6.82 (m, 2H, ArH), 6.88 (d, 1H, J=8.2 Hz, ArH)

### Procedure B

### Ethyl 3-(4-hydroxy-3-methoxyphenyl)propanoate (18) and Ethyl 5-(4-hydroxy-3-methoxyphenyl)pentanoate (19)

Intermediate 16 (1.0 g, 4.45 mmol) was dissolved in ethanol (10 mL) in a round bottom flask and 10% Pd/C (0.10 g, 10 wt %) was added to it. The reaction flask was degassed and then stirred under hydrogen atmosphere for 2 h at room temperature. After the completion of reaction, the mixture was diluted with ethanol (20 mL) and passed through a short bed of celite. The organic layer was concentrated to afford compound 18 (0.95 gm) as colorless oil in quantitative yield which was pure enough for the next step. ¹H NMR (400 MHz, CDCl₃) δ ppm 1.23 (t, 3H, J=7.2 Hz, CH₃), 2.58 (t, 2H, J=8.0 Hz, CH₂), 2.87 (t, 2H, J=7.6 Hz, CH₂), 3.84 (s, 3H, OCH₃), 4.12 (q, 2H, J=7.2 Hz, OCH₂), 6.68 (m, 2H, ArH), 6.82 (d, 1H, J=7.2 Hz, ArH).

Intermediate 17 (1.0 g, 3.96 mmol) was stirred under hydrogen atmosphere following procedure B to yield compound 19 (0.98 g) in quantitative yield which was taken to the next step without further purification. ¹H NMR (400 MHz, CDCl₃) δ ppm 1.24 (t, 3H, J=7.2 Hz, CH₃), 1.58-1.70 (m, 4H, CH₂), 2.31 (t, 2H, J=7.2 Hz, CH₂), 2.55 (t, 2H, J=7.2 Hz, CH₂), 3.87 (s, 3H, OCH₃), 4.12 (q, 2H, J=7.2 Hz, OCH₂), 6.65-6.67 (m, 2H, ArH), 6.82 (d, 1H, J=8.0 Hz, ArH)

### Procedure C

### 3-(4-Hydroxy-3-methoxyphenyl)propanal (20) and 5-(4-Hydroxy-3-methoxyphenyl)pentanal (21)

To a solution of compound 18 (0.60 g, 2.67 mmol) in dry toluene (10 mL) under argon was added DIBALH solution (1M in hexane, 2.93 mL, 2.93 mmol) at −78° C. The reaction mixture was stirred at −78 C for 2 h. Methanol (0.20 mL) was added to the reaction mixture and reaction allowed to come to 0° C. The reaction mixture was then added to a separating funnel containing HCl (1N, 10 ml) and extracted with ethyl acetate (3×30 mL). The combined organic layer was washed with brine, dried over sodium sulfate, and evaporated under reduced pressure. The crude was purified by column chromatography (Hexane/ethyl acetate 7:1) to give compound 20 (0.22 gm, 45.6%) as yellow oil. ¹H NMR (400 MHz, CDCl₃) δ ppm 2.75 (t, 2H, J=8.0 Hz, CH₂), 2.89 (t, 2H, J=7.6 Hz, CH₂), 3.87 (s, 3H, OCH₃), 6.68 (m, 2H, ArH), 6.83 (d, 1H, J=7.2 Hz, ArH), 9.81 (t, 1H, J=1.6 Hz, CHO)

Intermediate 19 (0.50 g, 1.96 mmol) was reduced with DIBALH solution (1M in hexane, 2.16 mL, 2.16 mmol) in dry tolune using procedure C. The crude was purified by column chromatography (Hexane/ethyl acetate 8:1) to yield compound 21 (0.196 g, 48%) as colorless oil. ¹H NMR (400 MHz, CDCl₃) δ ppm 1.59-1.68 (m, 4H, CH₂), 2.44 (td, 2H, J₁=6.40 Hz, J₂=1.60 Hz CH₂), 2.56 (t, 2H, J=7.2 Hz, CH₂), 3.87 (s, 3H, OCH₃), 6.65 (m, 2H, ArH), 6.82 (d, 1H, J=8.0 Hz, ArH), 9.75 (t, 1H, J=1.6 Hz, CHO).

### Procedure D

### 4-(3-((2-Amino-4,5,6,7-tetrahydrobenzo[d]thiazol-6-yl)(propyl)amino)propyl)-2-methoxyphenol (22) and 4-(5-((2-Amino-4,5,6,7-tetrahydrobenzo[d]thiazol-6-yl)(propyl)amino)pentyl)-2-methoxyphenol (23)

Into a stirring solution of (+) Pramipexole (0.123 g, 0.58 mmol) in DCE (15 mL) was added aldehyde 20 (0.10 g, 0.55 mmol) and the mixture stirred for 1 h. NaBH(OAC)₃ (0.235 g 1.10 mmol) was then added portion-wise and the reaction stirred for 24 h at room temperature. The reaction mixture was quenched with a saturated NaHCO₃ solution at 0° C. and extracted with ethyl acetate (3×25 mL). The combined organic layer was dried over sodium sulfate and solvent was removed under reduced pressure. The crude product was purified by column chromatography (ethyl acetate/methanol 20:1) to give compound 22 (0.079 g, 38%). ¹H NMR (400 MHz, CD₃OD) δ ppm 0.95 (t, 3H, J=7.2 Hz, CH₃), 1.59 (m, 2H, CH₂), 1.81-1.92 (m, 3H, CH₂), 2.05-2.12 (m, 1H, CH₂), 2.57-2.67 (m, 6H, CH₂), 2.73-2.89 (m, 5H, CH₂, CH), 3.82 (s, 3H, OCH₃), 6.64 (d, 1H, J=8.0 Hz, ArH), 6.71 (d, 1H, J=8.0 Hz, ArH), 6.79 (s, 1H, ArH). The free base was converted into its corresponding hydrochloride salt. Mp 220-222° C. Anal. (C₂₀H₃₂Cl₂N₃O_{2.5}S) C, H, N.

Aldehyde 21 (0.10 g, 0.48 mmol) and (±) Pramipexole (0.106 g, 0.50 mmol) in DCE (15 mL) were reacted using procedure D and the resulting crude was purified by column chromatography (ethyl acetate/methanol 21:1) to give compound 23 (0.068 g, 35%). ¹H NMR (400 MHz, DMSO-d₆) δ ppm 0.89 (t, 3H, J=7.2 Hz, CH₃), 1.28-1.36 (m, 3H, CH₂), 1.48-1.53 (m, 4H, CH₂), 1.59-1.70 (m, 3H, CH₂), 2.57-2.67 (m, 1H, CH₂), 2.503-2.65 (m, 9H, CH₂), 3.05 (bs, 1H, CH), 3.82 (s, 3H, OCH₃), 6.58 (d, 1H, J=8.4 Hz, ArH), 6.64-6.72 (m, 2H, ArH). The free base was converted into its corresponding hydrochloride salt. Mp 224-226° C. Anal. (C_{23.6}H_{37.8}N_{32.8}S_{1.22}) C, H, N.

Elemental Analysis Report of Compounds 22 and 23

| | Calculated | | | Found | | |
|---|---|---|---|---|---|---|
| Compound | C | H | N | C | H | N |
| 22. 2HCl•0.5H₂O | 52.51 | 7.05 | 9.19 | 52.12 | 7.19 | 8.83 |
| 23. 2HCl•0.58C₂H₅OH•0.22CH₃SOCH₃ | 54.47 | 7.71 | 8.07 | 54.86 | 7.31 | 7.60 |

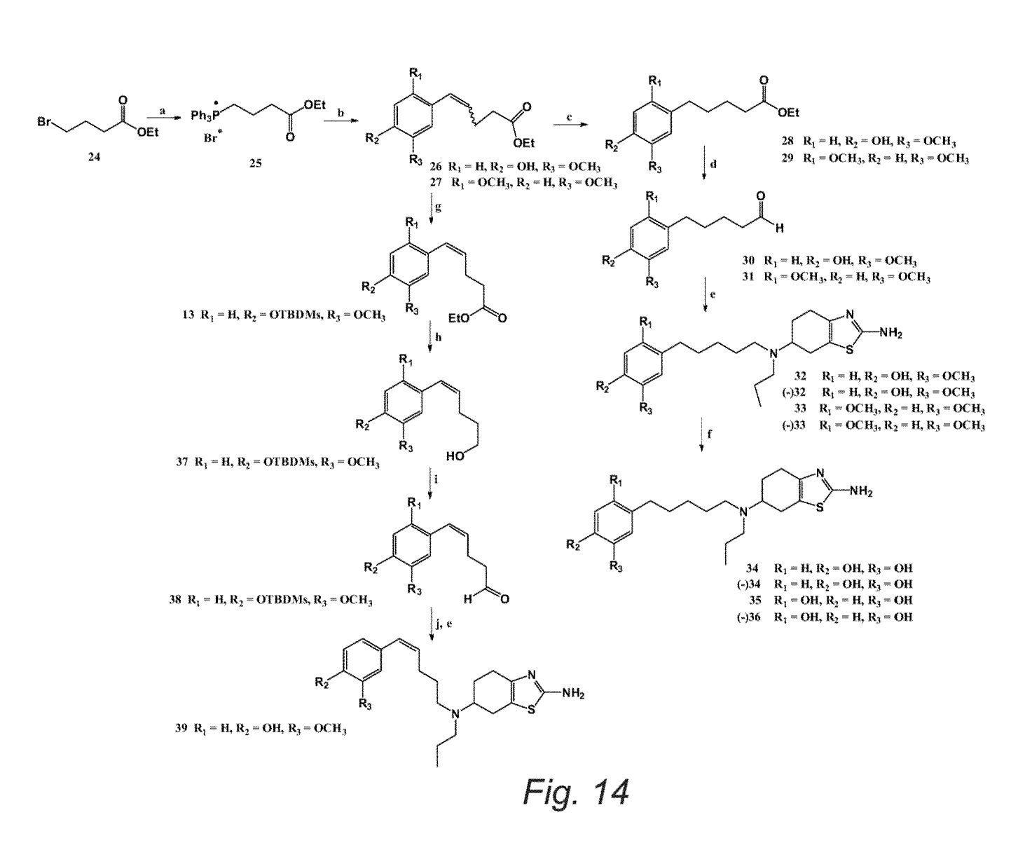

FIG. 14 provides a synthetic scheme for the compounds having formula II, specifically compounds 34, 35, and 35. The reaction conditions for this scheme are (a) TPP, toluene, reflux, 16 h, qunat. yield; (b) Aldehyde, NaHMDS, THF, −78° C. to rt, 48 h; 35-43%; (c) 10% pd/C, H₂, ethanol, rt, 2 h, 96%; (d) DIBALH, toluene, −78° C., 2 h, 48-80%; (e) Pramipexole, NaBH(OAc)₃, DCM, rt, 36 h, 53-69%; (f) BBr₃, DCM, −78° C. to rt, 6 h, 62-80%; (g) TBDMSCl, imidazole, DMF, rt, 2 h, qunat. yield; (h) DIBALH, THF, −10° C. to rt, 6 h, 87%; (i) PCC, DCM, ° C. to rt, 9 h, 45%; (j) TBAF, THF, 0° C., 1.5 h.

### [3-(Ethoxycarbonyl)propyl]triphenylphosphonium bromide (25)

### Procedure A′. (4-ethoxy-4-oxobutyl)triphenylphosphonium bromide

Triphenylphosphine (3.76 g, 14.35 mmol) and ethyl 4-bromobutyrate (2 g, 10.25 mmol) was added to a dried round bottom flask under argon. The reaction mixture was heated to 120° C. under condenser for 16 h after which the reaction was allowed to come to room temperature. DCM (10 mL) was added, followed by diethyl ether until no further precipitation of product was observed. The precipitate was further washed with ether (100 mL) and solvent dried in vacuo to give pure compound 25 as a white solid (4.64 g) in quantitative yields. ¹H NMR (400 MHz, CDCl₃) δ ppm 7.67-7.90 (m, 15H), 3.98-4.12 (m, 4H), 2.86-2.89 (m, 2H), 1.88-1.94 (m, 2H), 1.22 (t, J=7.2 Hz, 3H).

### Procedure B′. Ethyl 5-(4-hydroxy-3-methoxyphenyl)pent-4-enoate (26)

Starting material 2 (6.61 g, 14.46 mmol) was added to dry THF (15 mL) in an oven-dried round bottom flask. The resulting suspension was cooled to −78° C. Then a solution of NaHMDS (1M in THF, 15.77 mL, 15.77 mmol) was added drop-wise and the reaction mixture stirred at −78° C. for 1 h. Thereafter, a solution of Vanillin (2.0 g, 13.14 mmol) in dry THF (5 mL) was added drop-wise and reaction stirred at −78° C. for 2 h, then warmed to room temperature and stirred for 48 h. The reaction mixture was then extracted with ethyl acetate (2×100 mL) and washed with brine. The combined organic layer was dried over sodium sulfate and concentrated in vacuo. The crude thus obtained was purified by column chromatography using 10% ethyl acetate in hexane to yield compound 26 (1.17 g, 35.6%) as colorless oil with a preferential Z:E ratio of >20:1: ¹H NMR (400 MHz, CDCl₃) δ ppm 6.88 (d, J=8.2 Hz, 1H), 6.79-6.82 (m, 2H), 6.37 (d, J=11.2 Hz, 1H), 5.64 (s, 1H), 5.49-5.55 (m, 1H), 4.13 (q, J=7.2 Hz, 2H), 3.88 (s, 3H), 2.63-2.69 (m, 2H), 2.45 (t, J=8.0 Hz, 2H), 1.24 (t, J=7.2 Hz, 3H).

### Ethyl 5-(2,5-dimethoxyphenyl)pentanoate (27)

Starting material 25 (7.56 g, 16.57 mmol) was reacted with 2,5-dimethoxy benzaldehyde (2.5 g, 15.07 mmol) in presence of NaHMDS 1M in THF (18.05 mmol, 18.05 mL) according to Procedure A′. the crude was purified by column chromatography using 7-10% ethyl acetate in hexanes to give compound 27 (1.71 g, 43%). ¹H NMR (400 MHz, CDCl₃) δ ppm 6.82 (d, J=2.4 Hz, 1H), 6.74-6.79 (m, 2H), 6.52 (d, J=11.2 Hz, 1H), 5.65-5.72 (m, 1H), 4.12 (q, J=7.2 Hz, 2H), 3.78 (s, 3H), 3.77 (s, 3H), 2.55-2.60 (m, 2H), 2.41 (t, J=7.2 Hz, 2H), 1.23 (t, J=7.2 Hz, 3H).

### Procedure C′. Ethyl 5-(4-hydroxy-3-methoxyphenyl)pentanoate (5)

Intermediate 26 (1.0 g, 3.96 mmol) dissolved in ethanol (10 mL) in a round bottom flask and 10% Pd/C (0.10 g, 10 wt %) was added to it. The reaction flask was degassed and then stirred under hydrogen atmosphere for 2 h at room temperature. After the completion of reaction, the mixture was diluted with ethanol (20 mL) and passed through a short bed of celite. The organic layer was concentrated to afford compound 5(0.98 gm) in quantitative yield which was pure enough for the next step. ¹H NMR (400 MHz, CDCl₃) δ ppm 6.82 (d, J=8.0 Hz, 1H), 6.65-6.67 (m, 2H), 4.12 (q, J=7.2 Hz, 2H), 2.55 (t, J=7.2 Hz, 2H), 3.87 (s, 3H), 2.31 (t, J=7.2 Hz, 2H), 1.58-1.70 (m, 4H), 1.24 (t, J=7.2 Hz, 3H).

### Ethyl 5-(2,5-dimethoxyphenyl)pentanoate (29)

Intermediate 27 (1.0 g, mmol) dissolved in ethanol (10 mL) and reacted with 10% Pd/C (0.1 g, 10 wt %) according to Procedure C′ to afford compound 6 (0.96 g, 96%) which was used without further purification. ¹H NMR (400 MHz, CDCl₃) δ ppm 6.75 (d, J=8.8 Hz, 1H), 8.72 (d, J=3.2 Hz, 1H), 6.68 (dd, J=8.4, 2.4 Hz, 1H), 4.12 (q, J=7.2 Hz, 2H), 3.76 (s, 3H), 3.75 (s, 3H), 2.60 (t, J=7.4 Hz, 2H), 2.33 (t, J=7.2 Hz, 2H), 1.56-1.70 (m, 4H), 1.24 (t, J=7.2 Hz, 3H).

### Procedure D′. 5-(4-Hydroxy-3-methoxyphenyl)pentanal (30)

To a solution of compound 5 (0.50 g, 1.96 mmol) in dry toluene (10 mL) under argon was added DIBALH solution (1M in hexane, 2.16 mL, 2.16 mmol) at −78° C. The reaction mixture was stirred at −78 C for 2 h. Methanol (0.20 mL) was added to the reaction mixture and reaction allowed to come to 0° C. The reaction mixture was then added to a separating funnel containing HCl (1N, 10 ml) and extracted with ethyl acetate (3×30 mL). The combined organic layer was washed with brine, dried over sodium sulfate, and evaporated under reduced pressure. The crude was purified by column chromatography using 11-15% ethyl acetate in hexanes to yield compound 30 (0.196 g, 48%) as colorless oil. ¹H NMR (400 MHz, CDCl₃) δ ppm 9.75 (t, J=1.6 Hz, 1H), 6.82 (d, J=8.0 Hz, 1H), 6.65 (m, 2H), 3.87 (s, 3H), 2.56 (t, J=7.2 Hz, 2H), 2.44 (td, J₁=6.40 Hz, J₂=1.60 Hz 2H), 1.59-1.68 (m, 4H).

### 5-(2,5-dimethoxyphenyl)pentanal (31)

Intermediate 29 (0.6 g, 2.25 mmol) was reduced with DIBALH solution (1M in hexane, 2.47 mL, 2.47 mmol) in dry tolune (10 mL) using procedure D′. The crude was purified by column chromatography using 11-15% ethyl acetate in hexanes to give compound 8 (0.4 μm, 80%) as yellow oil. ¹H NMR (400 MHz, CDCl₃) δ ppm 9.74 (d, J=1.6 Hz, 1H), 6.76 (d, J=8.8 Hz, 1H), 6.67-6.71 (m, 2H), 3.76 (s, 3H), 3.75 (s, 3H), 2.60 (t, J=6.4 Hz, 2H), 2.44 (t, J=6.8 Hz, 2H), 1.58-1.37 (m, 4H).

### Procedure E′. 4-(5-((2-Amino-4,5,6,7-tetrahydrobenzo[d]thiazol-6-yl)(propyl)amino)pentyl)-2-methoxyphenol (32)

Into a stirring solution of (+) Pramipexole (015 g, 0.71 mmol) in DCM (8 mL) was added aldehyde 30 (0.148 g, 0.71 mmol) and the mixture stirred for 1 h. NaBH(OAc)₃ (0.27 g, 1.27 mmol) was then added portion-wise followed by MeOH (0.8 mL). The reaction was stirred for 36 h at room temperature. The reaction mixture was quenched with a saturated NaHCO₃ solution at 0° C. and extracted with DCM (3×25 mL). The combined organic layer was dried over sodium sulfate and solvent was removed under reduced pressure. The crude product was purified by column chromatography using 5-7% MeOH in DCM to give compound 32 (0.2 g, 69%). ¹H NMR (400 MHz, DMSO-d₆) δ ppm 6.64-6.72 (m, 2H), 6.58 (d, J=8.4 Hz, 1H), 3.82 (s, 3H), 3.05 (bs, 1H), 2.50-2.67 (m, 10H), 1.59-1.70 (m, 3H), 1.48-1.53 (m, 4H), 1.28-1.36 (m, 3H), 0.89 (t, J=7.2 Hz, 3H). The free base was converted into its corresponding hydrochloride salt. Mp 224-226° C. Anal. (C_{23.6}H_{37.8}N₃O_{2.8}S_{1.22}) C, H, N.

### (R)-4-(5-((2-Amino-4,5,6,7-tetrahydrobenzo[d]thiazol-6-yl)(propyl)amino)pentyl)-2-methoxyphenol (−32)

Aldehyde 30 (0.148 g, 0.71 mmol), (−) Pramipexole (0.15 g, 0.71 mmol), and NaBH(OAc)₃ (0.27 g, 1.27 mmol) in DCM (8 mL) and MeOH (0.8 mL) were reacted using procedure D′ and the resulting crude was purified by column chromatography using 5-7% MeOH in DCM to give compound (−32) (0.19 g, 66%). ¹H NMR (400 MHz, DMSO-d₆) δ ppm 6.64-6.72 (m, 2H), 6.58 (d, J=8.4 Hz, 1H), 3.82 (s, 3H), 3.05 (bs, 1H), 2.65-2.67 (m, 1H), 2.50-2.65 (m, 9H), 1.59-1.70 (m, 3H), 1.48-1.53 (m, 4H), 1.28-1.36 (m, 3H), 0.90 (t, J=7.2 Hz, 3H). The free base was converted into its corresponding hydrochloride salt. Mp 196-198° C. Anal. (C₂₂H₃₇N₃Cl₂O₃S) C, H, N.

### N⁶-(5-(2,5-dimethoxyphenyl)pentyl)-N⁶-propyl-4,5,6,7-tetrahydrobenzo[d]thiazole-2,6-diamine (33)

Aldehyde 31 (0.157 g, 0.706 mmol), (+) Pramipexole (0.15 g, 0.709 mmol), and NaBH(OAc)₃ (0.27 g, 1.27 mmol) in DCM (9 mL) and MeOH (0.8 mL) were reacted using procedure D′ and the resulting crude was purified by column chromatography using 5-7% MeOH in DCM to give compound (0.17 g, 57%). ¹H NMR (400 MHz, CDCl₃) δ ppm 6.75 (d, J=8.8 Hz, 1H), 6.70 (d, J=2.4 Hz, 1H), 6.66 (dd, J=8.8, 2.1 Hz, 1H), 3.75 (s, 3H), 3.74 (s, 3H), 3.07 (bs, 1H), 2.69 (dd, J=12.0, 4.04 Hz, 2H), 2.47-2.58 (m, 8H), 1.99-2.03 (m, 1H), 1.68-1.78 (m, 1H), 1.40-1.61 (m, 6H), 1.31-1.37 (m, 2H), 0.89 (t, J=7.2 Hz, 3H).

### (R)—N⁶-(5-(2,5-dimethoxyphenyl)pentyl)-N⁶-propyl-4,5,6,7-tetrahydrobenzo[d]thiazole-2,6-diamine (−33)

Aldehyde 31 (0.105 g, 0.473 mmol), (−) Pramipexole (0.10 g, 0.473 mmol), and NaBH(OAc)₃ (0.27 g, 0.849 mmol) in DCM (7 mL) and MeOH (0.6 mL) were reacted using procedure D′ and the resulting crude was purified by column chromatography using 5-7% MeOH in DCM to give compound (0.105 g, 53%). ¹H NMR (600 MHz, CDCl₃) δ ppm 6.76 (d, J=8.4 Hz, 1H), 6.71 (d, J=1.8 Hz, 1H), 6.67 (dd, J=8.4, 3.0 Hz, 1H), 4.71 (bs, 2H), 3.77 (s, 3H), 3.76 (s, 3H), 3.04 (bs, 1H), 2.69 (d, J=7.2 Hz, 2H), 2.4-2.6 (m, 8H), 2.0 (bs, 1H), 1.62-1.72 (m, 2H), 1.55-1.61 (m, 2H), 1.41-1.52 (m, 3H), 1.33-1.37 (m, 2H), 0.88 (t, J=7.2 Hz, 3H).

### Procedure F′. 4-(5-((2-Amino-4,5,6,7-tetrahydrobenzo[d]thiazol-6-yl)(propyl)amino)pentyl)benzene-1,2-diol (34)

BBr₃ 1M in DCM (2.66 mL, 2.66 mmol) was added to a solution of compound 32 (0.2 g, 0.532 mmol) in DCM (20 mL) at −78° C. and stirred for 2 h. The reaction mixture was allowed to come to room temperature and stirred for another 4 h. The reaction was then quenched by methanol (20 mL) at 0° C. and the solvent was concentrated in vacuo, and MeOH (20 mL) was added to the residue and again evaporated. This process was repeated three times. The residue obtained was purified by column chromatography using 10-15% methanol in DCM to give pure compound 34 (123 mg, 64%). ¹H NMR (400 MHz, CD₃OD) δ ppm 6.64 (d, J=8.0 Hz, 1H), 6.60 (d, J=2.4 Hz, 1H), 6.47 (dd, J=8.0, 2.4 Hz, 1H), 3.35-3.40 (m, 1H), 2.75-2.85 (m, 5H), 2.62-2.71 (m, 2H), 2.55-2.57 (m, 1H), 2.47 (t, J=7.2 Hz, 2H), 2.09-2.14 (m, 1H), 1.79-1.89 (m, 1H), 1.57-1.64 (m, 6H), 1.31-1.38 (m, 2H), 0.95 (t, J=7.6 Hz, 3H). The free base was converted into its corresponding hydrochloride salt. Mp 196-198° C. Anal. (C₂₁H₃₄N₃Cl₃O₂S) C, H, N.

### (R)-4-(5-((2-Amino-4,5,6,7-tetrahydrobenzo[d]thiazol-6-yl)(propyl)amino)pentyl)benzene-1,2-diol (−34)

BBr₃ 1M in DCM (1.61 mL, 1.61 mmol) was reacted with compound −32 (0.132 g, 0.327 mmol) in DCM (10 mL) according to Procedure F′ to afford crude which was purified by column chromatography using 10-15% MeOH in DCM to give compound −11 (0.09 g, 70%). ¹H NMR (600 MHz, CD₃OD) δ ppm 6.63 (d, J=7.8 Hz, 1H), 6.58 (d, J=1.8 Hz, 1H), 6.45 (dd, J=7.8, 1.8 Hz, 1H), 2.97-3.02 (m, 1H), 2.42-2.62 (m, 10H), 1.95-1.97 (m, 1H), 1.60-1.73 (m, 1H), 1.53-1.58 (quintet, J=7.8 Hz, 2H), 1.42-1.48 (septet, J=7.2 Hz, 4H), 1.26-1.31 (quintet, J=7.2 Hz, 2H), 0.88 (t, J=7.2 Hz, 3H). The free base was converted into its corresponding hydrochloride salt. Mp 211-212° C. Anal. (C₂₁H₃₅N₃Cl₂O₂S) C, H, N.

### 2-(5-((2-Amino-4,5,6,7-tetrahydrobenzo[d]thiazol-6-yl)(propyl)amino)pentyl)benzene-1,4-diol (35)

BBr₃ 1M in DCM (2.27 mL, 2.27 mmol) was reacted with compound 33 (0.16 g, 0.383 mmol) according to Procedure F′ to afford crude which was purified by column chromatography using 10-15% MeOH in DCM to afford compound 35 (0.12 g, 80%). ¹H NMR (600 MHz, CD₃OD) δ ppm 6.55 (d, J=8.4 Hz, 1H), 6.51 (d, J=3.0 Hz, 1H), 6.41 (dd, J=8.4, 3.0 Hz, 1H), 3.0 (m, 1H), 2.44-2.63 61 (m, 10H), 1.98 (d, J=10.2 Hz, 1H), 1.65-1.68 (m, 1H), 1.56-1.61 (m, 2H), 1.44-1.52 (m, 4H), 1.32-1.36 (m, 2H), 0.88 (t, J=7.2 Hz, 3H). The free base was converted into its corresponding hydrochloride salt. Mp 217-219° C. Anal. (C₂₁H_{35.8}N₃Cl_{32.9}S) C, H, N.

### (R)-2-(5-((2-Amino-4,5,6,7-tetrahydrobenzo[d]thiazol-6-yl)(propyl)amino)pentyl)benzene-1,4-diol (−35)

BBr₃ 1M in DCM (1.33 mL, 1.33 mmol) was reacted with compound (0.093 g, 0.222 mmol) in DCM (6 mL) according to Procedure F′ to afford crude which was purified by column chromatography using 10-15% MeOH in DCM to give compound −35 (62 g, 72%). ¹H NMR (600 MHz, CD₃OD) δ ppm 6.56 (dd, J=8.4, 1.8 Hz, 1H), 6.52 (d, J=2.4 Hz, 1H), 6.43 (dt, J=8.4, 1.80 Hz, 1H), 2.98-3.03 (m, 1H), 2.44-2.64 (m, 10H), 1.99-2.00 (m, 1H), 1.63-1.71 (m, 1H), 1.57-1.62 (m, 2H), 1.46-1.52 (m, 4H), 1.32-1.37 (m, 2H), 0.90 (t, J=7.2 Hz, 3H). The free base was converted into its corresponding hydrochloride salt. Mp 222-224° C. Anal. (C₂₁H₃₄N₃Cl₃O₂S) C, H, N.

### Procedure G′. (Z)-ethyl 5-(4-((tert-butyldimethylsilyl)oxy)-3-methoxyphenyl)pent-4-enoate (36)

Imidazole (0.411 g, 6.03 mmol) was added to a solution of intermediate 26 (0.61 g, 2.41 mmol) and tert-butyldimethylsilyl chloride (0.437 g, 2.89 mmol) in DMF (5 mL). The solution was stirred at room temperature for 2 h. After the completion of reaction, saturated aq NaHCO₃ (10 mL) was added and stirred for another 30 min. The reaction mixture was extracted with DCM (2×50 mL). The combined organic layer was dried over sodium sulfate and concentrated in vacuo. The crude thus obtained was purified by column chromatography using 5% ethyl acetate in hexanes to afford compound (0.88 g) in quantitative yield. ¹H NMR (400 MHz, CD₃OD) δ ppm 6.85 (s, 1H), 6.79 (d, J=8.0 Hz, 1H), 6.73 (d, J=8.4 Hz, 1H), 4.37 (d, J=11.2 Hz, 1H), 5.47-5.55 (m, 1H), 4.10 (m, J=7.2 Hz, 2H), 3.78 (s, 3H), 2.63 (q, J=7.2 Hz, 2H), 2.44 (t, J=7.2 Hz, 2H), 1.20 (t, J=7.2 Hz, 3H), 0.99 (s, 9H), 0.13 (s, 6H).

### Procedure H′. (Z)-5-(4-((tert-butyldimethylsilyl)oxy)-3-methoxyphenyl)pent-4-en-1-ol (37)

Into a stirring solution of compound (0.86 g, 2.34 mmol) in anhydrous THF (15 mL) was added DIBAL-H solution 1 M in THF (11.8 mL, 11.8 mmol) dropwise at −10° C. The reaction mixture was stirred at room temperature for 6 h and was quenched with methanol (1 mL). The mixture was acidified to neural with 1N HCl (15 mL) solution. The organic layer was separated and the aqueous layer was extracted with ethyl acetate (2×100 mL). The combined organic layer was dried over sodium sulfate and concentrated in vacuo. The crude product was purified by column chromatography using 11-15% ethyl acetate in hexanes to give compound (0.63 g, 87%). ¹H NMR (CDCl₃, 400 MHz) δ ppm 6.74-6.85 (m, 3H), 6.37 (d, J=11.2 Hz, 1H), 5.48-5.53 (m, 1H), 3.80 (s, 3H), 2.67 (q, J=7.2 Hz, 2H), 2.43 (t, J=7.2 Hz, 2H), 1.22 (t, J=7.2 Hz, 2H), 1.06 (s, 9H), 0.15 (s, 6H).

### Procedure I′. (Z)-5-(4-((tert-butyldimethylsilyl)oxy)-3-methoxyphenyl)pent-4-enal (38)

Alcohol (0.9 g, 2.46 mmol) was slowly added to a ice cooled suspension of PCC (0.707 g, 3.28 mmol) in DCM (90 mL) and the mixture was stirred at rt for 9 h. The reaction mixture was then filtered over celite and solvent was evaporated to get residue which was purified by column chromatography using 10-15% of ethyl acetate in hexanes to give compound 15 (0.358 g, 45%). ¹H NMR (CDCl₃, 400 MHz) δ ppm 9.78 (s, 1H), 6.74-6.85 (m, 3H), 6.37 (d, J=11.2 Hz, 1H), 5.49-5.52 (m, 1H), 3.80 (s, 3H), 2.56-2.68 (m, 4H), 0.99 (s, 9H), 0.16 (s, 6H).

### (Z)-4-(5-((2-amino-4,5,6,7-tetrahydrobenzo[d]thiazol-6-yl)(propyl)amino)pent-1-en-1-yl)-2-methoxyphenol (39)

Compound 38 (0.315 g, 0.982 mmol) was dissolved in THF (5 mL) and cooled to 0° C. TBAF 1M in THF (1.02 mL, 1.02 mmol) was added and reaction stirred in ice for 1.5 h. After reaction was complete, 10% NaHCO₃ solution (10 mL) was added and reaction mixture was extracted with DCM (3×50 mL) to obtain aldehyde which was immediately taken to the next step without further purification. Aldehyde (0.195 g, 0.945 mmol), (+) Pramipexole (0.20 g, 0.946 mmol), and NaBH(OAC)₃ (0.36 g, 1.69 mmol) in DCM (7 mL) and MeOH (1.0 mL) were reacted using procedure D′ and the resulting crude was purified by column chromatography using 5-7% MeOH in DCM to give compound 39 (0.148 g, 39%). ¹H NMR (400 MHz, CD₃OD) δ ppm 6.86 (d, J=8.0 Hz, 1H), 6.76-6.82 (m, 2H), 6.35 (d, J=12.4 Hz, 1H), 5.49-5.56 (m, 1H), 4.98 (bs, 1H), 3.86 (s, 3H), 3.1 (bs, 1H), 2.50-2.71 (m, 8H), 2.37 (q, J=7.2 Hz, 2H), 2.03 (bs, 1H), 1.44-1.70 (m, 5H), 0.88 (t, J=7.2 Hz, 3H). The free base was converted into its corresponding hydrochloride salt. Mp 212-214° C. Anal. (C₂₂H_{34.4}N₃C₁₃O_{2.2}S) C, H, N.

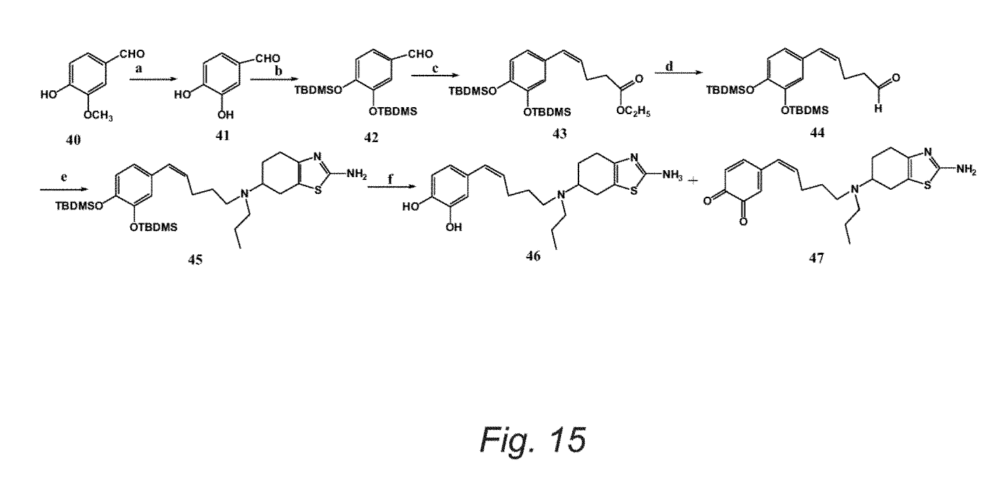

FIG. 15 provides a synthetic scheme for the compounds having formula II, specifically for compound 47. The reaction conditions for this scheme are: (a) BBr₃, DCM, −78° C. to rt, 6 h, 87%; (b) TBDMSCl, imidazole, DMF, rt, 2 h, 67%; (c) Phosphonium bromide 2, THF, −78° C. to rt, 48 h; 39%; (d) DIBALH, toluene, −78° C., 2 h, 45%; (e) Pramipexole, NaBH(OAc)₃, DCM, rt, 36 h, 50%; (f) TBAF, THF, 0° C., 1.5 h, 72%.

### 3,4-dihydroxybenzaldehyde (41)

Vanillin 40 (3.0 g, 18.0 mmol) was reacted with BBr₃ 1M in DCM (36.1 mL, 36.1 mmol) in DCM (20 mL) following Procedure F′. The crude obtained was purified by column chromatography using 20-25% ethyl acetate in hexanes to afford compound 41 (2.18 g, 87%). ¹H NMR (400 MHz, CDCl₃) δ ppm 9.67 (s, 1H, CHO), 7.29 (m, 2H), 6.89 (d, J=9.2 Hz, 1H).

### 3,4-bis((tert-butyldimethylsilyl)oxy)benzaldehyde (42)

Imidazole (5.69 g, 83.5 mmol) was added to a solution of intermediate 41 (2.31 g, 16.7 mmol) and tert-butyldimethylsilyl chloride (6.30 g, 41.8 mmol) in DMF (20 mL). The solution was reacted using Procedure G′. The crude thus obtained was purified by column chromatography using 5% ethyl acetate in hexanes to afford compound 42 (4.10 g, 67%). ¹H NMR (400 MHz, CDCl₃) δ ppm 9.80 (s, 1H, CHO), 7.36 (m, 2H), 6.93 (d, J=8.8 Hz, 1H), 0.99 (s, 9H), 0.98 (s, 9H), 0.24 (s, 6H), 0.22 (s, 6H).

### (Z)-ethyl 5-(3,4-bis((tert-butyldimethylsilyl)oxyphenyl)pent-4-enoate (43)

Starting material 25 (5.48 g, 11.98 mmol) was reacted with compound 42 (4.0 g, 10.90 mmol) in presence of NaHMDS 1M in THF (13.07 mmol, 13.07 mL) according to Procedure A′. The crude was purified by column chromatography using 5% ethyl acetate in hexanes to give compound 20 (2.0 g, 39%). ¹H NMR (400 MHz, CDCl₃) δ ppm 6.81 (m, 2H), 6.73 (d, J=8.4 Hz, 1H), 6.30 (d, J=11.4 Hz, 1H), 5.46-5.50 (m, 1H), 4.12 (q, J=7.3 Hz, 2H), 2.64 (q, J=7.2 Hz, 2H), 2.41 (t, J=7.2 Hz, 2H), 1.24 (t, J=7.2 Hz, 3H), 0.97 (s, 18H), 0.19 (s, 6H), 0.18 (s, 6H).

### (Z)-ethyl 5-(3,4-bis((tert-butyldimethylsilyl)oxyphenyl)pent-4-enal (44)

Intermediate 43 (1.0 g, 2.15 mmol) was reduced with DIBALH solution 1M in hexane (2.16 mL, 2.16 mmol) in dry tolune (15 mL) using procedure D′. The crude was purified by column chromatography using 10% ethyl acetate in hexane to give compound 44 (0.41 gm, 45%) as yellow oil. ¹H NMR (600 MHz, CDCl₃) δ ppm 9.76 (s, 1H), 6.77-6.79 (m, 2H), 6.73 (td, J=7.2, 2.4 Hz, 1H), 6.33 (d, J=11.4 Hz, 1H), 5.45-5.50 (m, 1H), 2.65 (q, J=7.2 Hz, 2H), 2.56 (t, J=7.2 Hz, 2H), 0.98 (s, 18H), 0.20 (s, 12H).

### (Z)—N⁶-(5-(3,4-bis((tert-butyldimethylsilyl)oxy)phenyl)pent-4-en-1-yl)-N⁶-propyl-4,5,6,7-tetrahydrobenzo[d]thiazole-2,6-diamine (45)

Aldehyde 44 (0.3 g, 0.71 mmol), (+) Pramipexole (0.15 g, 0.71 mmol), and NaBH(OAc)₃ (0.27 g, 1.27 mmol) in DCM (10 mL) and MeOH (0.8 mL) were reacted using procedure E′ and the resulting crude was purified by column chromatography using 5% MeOH in DCM to give compound 45 (0.22 g, 50%). ¹H NMR (600 MHz, CD₃OD) δ ppm 6.79 (m, 2H), 6.73 (d, J=7.8 Hz, 1H), 6.29 (d, J=11.4 Hz, 1H), 5.52 (m, 1H), 3.09 (bs, 1H), 2.44-2.66 (m, 8H), 2.34-2.37 (m, 2H), 1.96-1.99 (m, 1H), 1.60-1.72 (m, 3H), 1.44-1.51 (m, 2H), 0.97 (s, 18H), 0.87 (t, J=7.2 Hz, 3H), 0.18 (s, 12H).

### Procedure J′. (Z)-4-(5-((2-amino-4,5,6,7-tetrahydrobenzo[d]thiazol-6-yl)(propyl)amino)pent-1-en-1-yl)benzene-1,2-diol and 4-(5-((2-amino-4,5,6,7-tetrahydrobenzo[d]thiazol-6-yl)(propyl)amino)pent-1-enyl)-[1,2]benzoquinone (mixture of 46 and 47)

Compound (0.21 g, 0.34 mmol) was dissolved in THF (5 mL) and cooled to 0° C. TBAF 1M in THF (1.02 mL, 1.02 mmol) was added and reaction stirred in ice for 1.5 h. After reaction was complete, 10% NaHCO₃ solution (10 mL) was added and reaction mixture was extracted with DCM (3×50 mL). The combined organic layer was dried over sodium sulfate and concentrated in vacuo. The crude was purified by column chromatography using 15-20% MeOH in DCM (0.095 g, 72%). ¹H NMR (600 MHz, CD₃OD) δ ppm 6.79 (s, 1H), 6.73 (s, 1H), 6.70 (d, J=7.8 Hz, 1H), 6.63 (m, 2H), 6.59 (d, J=8.4 Hz, 1H), 6.20-6.25 (m, 2H), 6.59 (dt, J=15.6, 7.2 Hz, 1H), 5.46 (m, 1H), 3.0 (bs, 2H), 2.40-2.63 (m, 16H), 2.32 (m, 2H), 2.16 (m, 2H), 1.95 (m, 2H), 1.56-1.70 (m, 6H), 1.41-1.51 (m, 4H), 0.85-0.90 (m, 6H). The free base was converted into its corresponding hydrochloride salt. Mp 212-214° C. Anal. (C₂₂H_{34.4}N₃Cl_{32.2}S) C, H, N.

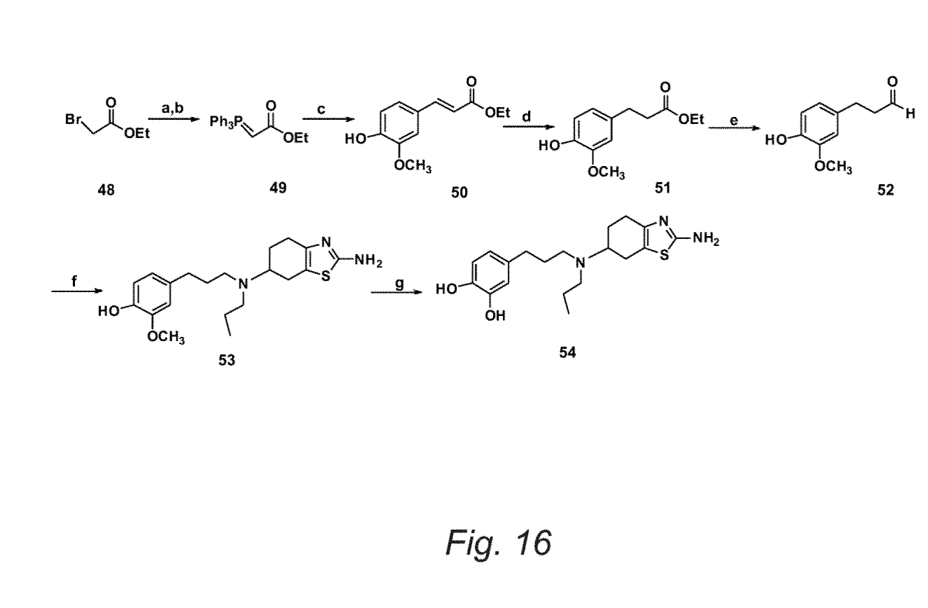

FIG. 16 provides a synthetic scheme for the compounds having formula II, specifically for compound 54. The reaction conditions for this scheme are: (a) TPP, toluene, reflux, 16 h, 87%; (b) 1 M NaOH aq., 15 min, 88%; (c) Vanillin, DCM, reflux, 5 h, 85% (d) 10% pd/C, H₂, ethanol, rt, 2 h, quant. yield; (d) DIBALH, toluene, −78° C., 2 h, 45%; (e) Pramipexole, NaBH(OAc)₃, DCM, rt, 36 h, 46%; (f) BBr₃, DCM 0° C.-rt, 6 h, 63%.

### (ethyl 2-(triphenylphosphoranylidene)acetate (49)

Ethyl bromoacetate (1.98 mL, 17.90 mmol) was slowly added to a stirring solution of triphenylphosphine (5.64 g, 21.50 mmol) in toluene (20 mL). The reaction mixture was refluxed for 16 h and filtered. The filter cake was washed with ethyl acetate (3×20 mL) to give phosphonium bromide (6.69 g, 87%). Into a solution of phosphonium bromide (6 g, 13.98 mmol) in DCM (50 mL) was added an aqueous solution of NaOH (1.0 M, 50 mL). The mixture was vigorously stirred for 15 min and the layers were separated. The aqueous layer was extracted with DCM (3×15 mL). The combined organic phase was dried over Na₂SO₄ and concentrated to give compound 49 (4.28 g, 88%). ¹H NMR (CDCl₃, 400 MHz): δ (0.65, minor+1.21, major) (t, J=7.2 Hz, 3H), (2.70, minor+2.92, major) (d, J=22 Hz, 1H), (3.80, minor+4.01, major) (q, J=7.2 Hz, 2H), 7.40-7.77 (m, 15H).

### Ethyl 3-(4-hydroxy-3-methoxyphenyl)acrylate (50)

Intermediate 49 (3.10 g, 7.23 mmol) was reacted with NaHMDS (1M in THF, 7.88 mL, 7.88 mmol) and Vanillin (1.0 g, 6.57 mmol) in anhydrous THF (8 mL) according to Procedure B′ The crude thus obtained was purified by column chromatography using 7-10% ethyl acetate in hexanes to yield compound 50 (1.25 g, 85%) as colorless oil with a preferential Z:E ratio of >20:1: ¹H NMR (400 MHz, CDCl₃) δ ppm 7.03 (m, ArH, 2H), 6.91 (d, J=8.0 Hz, ArH, 1H), 6.29 (d, J=15.2 Hz, 1H), 5.83 (m, 1H), 4.25 (q, J=7.2 Hz, OCH₂, 2H), 3.92 (s, OCH₃, 3H), 1.33 (t, J=7.6 Hz, 3H).

### Ethyl 3-(4-hydroxy-3-methoxyphenyl)propanoate (51)

Intermediate 50 (1.0 g, 4.45 mmol) was dissolved in ethanol (10 mL) in a round bottom flask and 10% Pd/C (0.10 g, 10 wt %) was added to it. The reaction was continued using Procedure C′. After the completion of reaction, the mixture was diluted with ethanol (20 mL) and passed through a short bed of celite. The organic layer was concentrated to afford compound 51 (0.95 gm) as colorless oil in quantitative yield which was pure enough for the next step. ¹H NMR (400 MHz, CDCl₃) δ ppm 6.82 (d, J=7.2 Hz, 1H), 6.68 (m, 2H), 4.12 (q, J=7.2 Hz, 2H), 3.84 (s, 3H), 2.87 (t, J=7.6 Hz, 2H), 2.58 (t, J=8.0 Hz, 2H), 1.23 (t, J=7.2 Hz, 3H).

### 3-(4-Hydroxy-3-methoxyphenyl)propanal (52)

Intermediate 51 (0.60 g, 2.67 mmol) was reduced with DIBALH solution (1M in hexane, 2.93 mL, 2.93 mmol) in dry tolune (10 mL) using procedure D′. The crude was purified by column chromatography using 12-15% ethyl acetate in hexanes to give compound 52 (0.22 gm, 45%) as yellow oil. ¹H NMR (400 MHz, CDCl₃) δ ppm 9.81 (t, J=1.6 Hz, 1H), 6.83 (d, J=7.2 Hz, 1H), 6.68 (m, 2H), 3.87 (s, 3H), 2.89 (t, J=7.6 Hz, 2H), 2.75 (t, J=8.0 Hz, 2H).

### 4-(3-((2-Amino-4,5,6,7-tetrahydrobenzo[d]thiazol-6-yl)(propyl)amino)propyl)-2-methoxyphenol (53)

Aldehyde 52 (0.30 g, 1.66 mmol), (+) Pramipexole (0.369 g, 1.74 mmol), and NaBH(OAc)₃ (0.633 g, 2.98 mmol) in DCM (10 mL) and MeOH (1 mL) were reacted using procedure E′ and the resulting crude was purified by column chromatography using 5-7% MeOH in DCM to give compound 53 (0.2 g, 46%). ¹H NMR (400 MHz, CD₃OD) δ ppm 6.79 (s, 1H), 6.71 (d, J=8.0 Hz, 1H), 6.64 (d, J=8.0 Hz, 1H), 3.82 (s, 3H), 2.73-2.89 (m, 5H), 2.57-2.67 (m, 6H), 2.05-2.12 (m, 1H), 1.81-1.92 (m, 3H), 1.59 (m, 2H), 0.95 (t, J=7.2 Hz, 3H). The free base was converted into its corresponding hydrochloride salt. Mp 220-222° C. Anal. (C₂₀H₃₂Cl₂N₃O_{2.5}S) C, H, N.

### 4-(3-((2-Amino-4,5,6,7-tetrahydrobenzo[d]thiazol-6-yl)(propyl)amino)propyl)-benzene-1,4-diol (54)

bbr₃1m in dcm (2.66 ml, 2.66 mmol) was reacted with compound 53 (0.2 g, 0.532 mmol) according to Procedure F′ to afford crude which was purified by column chromatography using 15-20% MeOH in DCM to provide compound 54 (0.12 g, 63%)¹H NMR (400 MHz, CD₃OD) δ ppm 6.57 (d, J=8.4 Hz, 1H), 6.53 (s, 1H), 6.41 (d, J=8.4 Hz, 1H), 3.13 (bs, 1H), 2.39-2.66 (m, 10H), 1.95 (m, 1H), 1.61-1.75 (m, 3H), 1.41-1.47 (m, 2H), 0.82 (t, J=7.2 Hz, 3H). The free base was converted into its corresponding hydrochloride salt. Mp 192-194° C. Anal. (C₁₉H_{30.6}N₃Cl_{32.3}S) C, H, N.

Elemental Analysis Report of Final Compounds

| | Calculated | | | Found | | |
|---|---|---|---|---|---|---|
| Compound | C | H | N | C | H | N |
| (9). | 4.47 | 7.71 | 8.07 | 4.86 | 7.31 | 7.60 |
| 2HCl•0.58C₂H₅OH•0.22CH₃SOCH₃ | | | | | | |
| (D-548) | | | | | | |
| (−9). 2HCl•H₂O | 53.43 | 7.34 | 8.75 | 51.97 | 7.25 | 4.41 |
| (11). 3HCl | 50.55 | 6.87 | 8.42 | 50.33 | 7.01 | 8.53 |
| (D-585) | | | | | | |
| (−11). 2HCl•H₂O | 52.49 | 7.34 | 8.75 | 51.97 | 7.25 | 8.41 |
| (12). 3HCl•0.9H₂O | 48.96 | 7.00 | 8.16 | 49.35 | 6.76 | 7.75 |
| (D-594) | | | | | | |
| (−12). 3HCl | 50.55 | 6.87 | 8.42 | 50.26 | 7.14 | 8.23 |
| (D-601) | | | | | | |
| (16). 3HCl•0.2H₂O | 51.35 | 6.74 | 8.17 | 51.65 | 7.16 | 7.63 |
| (D-567) | | | | | | |
| (23). 3HCl | 50.76 | 6.49 | 8.46 | 50.07 | 7.33 | 7.97 |
| (30). 2HCl•0.5H₂O | 52.51 | 7.05 | 9.19 | 52.12 | 7.19 | 8.83 |
| (D-547) | | | | | | |
| (31). 3HCl•0.3H2O | 47.91 | 6.48 | 8.82 | 47.86 | 6.87 | 9.23 |
| (D-583) | | | | | | |

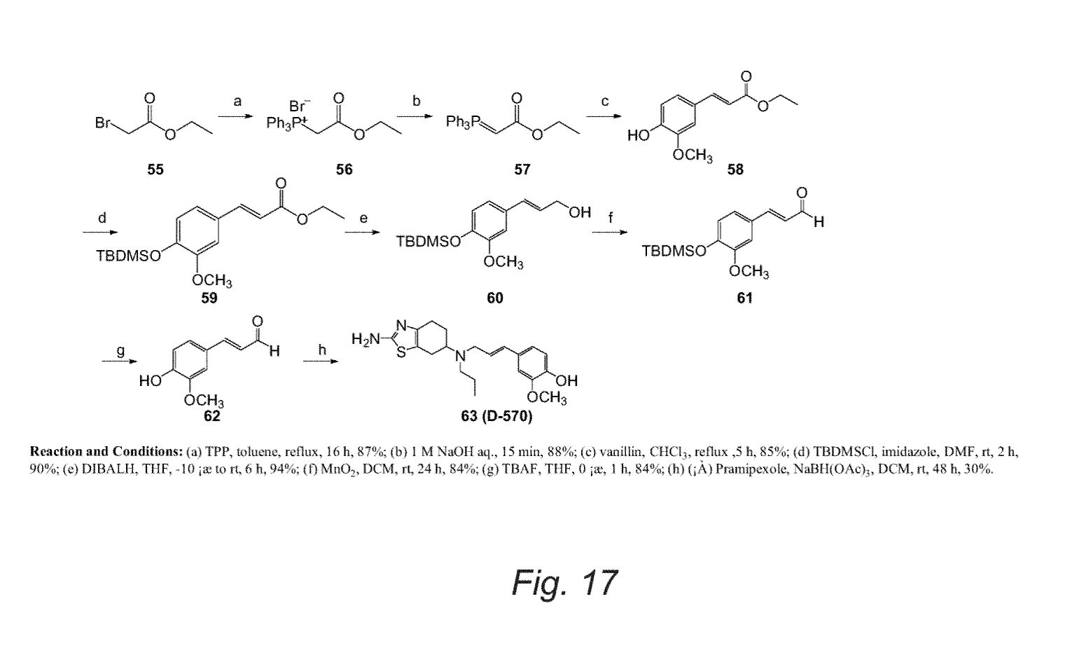

FIG. 17 provides a synthetic scheme for the compounds having formula II, specifically for compound 63. The conditions for this scheme are: (a) TPP, toluene, reflux, 16 h, 87%; (b) 1 M NaOH aq., 15 min, 88%; (c) vanillin, CHCl₃, reflux, 5 h, 85%; (d) TBDMSCl, imidazole, DMF, rt, 2 h, 90%; (e) DIBALH, THF, −10° C. to rt, 6 h, 94%; (f) MnO₂, DCM, rt, 24 h, 84%; (g) TBAF, THF, 0° C., 1 h, 84%; (h) (+) Pramipexole, NaBH(OAc)₃, DCM, rt, 48 h, 30%.

### (2-ethoxy-2-oxoethyl)triphenylphosphonium bromide (56)

Ethyl bromoacetate (1.98 mL, 17.90 mmol) was slowly added to a stirring solution of triphenylphosphine (5.64 g, 21.50 mmol) in toluene (20 mL). The reaction mixture was refluxed for 16 h and filtered. The filter cake was washed with ethyl acetate (3×20 mL) to give compound 2 (6.69 g, 87%). ¹H NMR (CDCl₃, 400 MHz): δ 1.04 (t, J=7.2 Hz, 3H), 4.01 (q, J=7.2 Hz, 2H), 5.54 (d, J=13.6 Hz, 2H), 7.64-7.91 (m, 15H).

### ethyl 2-(triphenylphosphoranylidene)acetate (57)

Into a solution of phosphonium bromide 54 (6 g, 13.98 mmol) in DCM (50 mL) was added an aqueous solution of NaOH (1.0 M, 50 mL). The mixture was vigorously stirred for 15 min and the layers were separated. The aqueous layer was extracted with DCM (3×15 mL). The combined organic phase was dried over Na₂SO₄ and concentrated to give compound 57 (4.28 g, 88%). ¹H NMR (CDCl₃, 400 MHz): δ (0.65, minor+1.21, major) (t, J=7.2 Hz, 3H), (2.70, minor+2.92, major) (d, J=22 Hz, 1H), (3.80, minor+4.01, major) (q, J=7.2 Hz, 2H), 7.40-7.77 (m, 15H).

### (E)-ethyl 3-(4-hydroxy-3-methoxyphenyl)acrylate (58)

A mixture of vanillin (1.65 g, 10.84 mmol) and phosphonium ylide 57 (4.08 g, 11.71 mmol) was refluxed in chloroform (30 mL) for 5 h. The solvent was removed and the crude product was purified by column chromatography (17% ethyl acetate in Hexane) to give compound 58 (2.05 g, 85%). ¹H NMR (CDCl₃, 400 MHz): δ 1.19 (t, J=7.2 Hz, 3H), 3.70 (s, 3H), 4.13 (q, J=7.0 Hz, 2H), 6.17 (d, J=15.7 Hz, 1H), 6.75-6.92 (m, 3H), 7.49 (d, J=15.9 Hz, 1H).

### (E)-ethyl 3-(4-((tert-butyldimethylsilyl)oxy)-3-methoxyphenyl)acrylate (59)

A solution of compound 58 (1.78 g, 8.01 mmol), tert-butyldimethylsilyl chloride (1.45 g, 9.62 mmol), and Imidazole (1.36 g, 19.98 mmol) in DMF (6 mL) was stirred at room temperature for 2 h. Then the mixture was extracted with water and diethyl ether. The organic phase was washed with brine and dried over Na₂SO₄. Then it was concentrated to give compound 5 (2.42 g, 90%). ¹H NMR (CDCl₃, 400 MHz): δ 0.17 (s, 6H), 0.99 (s, 9H), 1.33 (t, J=7.2 Hz, 3H), 3.83 (s, 3H), 4.25 (q, J=7.1 Hz, 2H), 6.30 (d, J=16.0 Hz, 1H), 6.84 (d, J=8.8 Hz, 1H), 6.99-7.03 (m, 2H), 7.62 (d, J=15.2 Hz, 1H).

### (E)-3-(4-((tert-butyldimethylsilyl)oxy)-3-methoxyphenyl)prop-2-en-1-ol (60)

Into a stirring solution of compound 59 (2.4 g, 7.13 mmol) in anhydrous THF (30 mL) was added DIBAL-H solution (1 M in THF, 35.7 mL, 35.7 mmol) dropwise at −10° C. The reaction mixture was stirred at room temperature for 6 h and was quenched with methanol (1 mL). The mixture was acidified to neural with 1N HCl solution. The organic layer was separated and the aqueous layer was extracted with ethyl acetate (2×20 mL). The combined organic phase was dried over Na₂SO₄ and concentrated. The crude product was purified by column chromatography (25% ethyl acetate in Hexane) to give compound 60 (1.97 g, 94%). ¹H NMR (CDCl₃, 400 MHz): δ 0.15 (s, 6H), 0.99 (s, 9H), 3.81 (s, 3H), 4.28 (d, J=5.6 Hz, 2H), 6.22 (dt, J=16.0, 6.0 Hz, 1H), 6.52 (d, J=15.6 Hz, 1H), 6.79 (d, J=8.0 Hz, 1H), 6.84 (dd, J=8.0, 1.6 Hz, 1H), 6.90 (d, J=1.6 Hz, 1H).

### (E)-3-(4-((tert-butyldimethylsilyl)oxy)-3-methoxyphenyl)acrylaldehyde (61)

MnO₂ (3.54 g, 40.72 mmol) was added into a solution of compound 60 (0.8 g, 2.72 mmol) in DCM (15 mL). The mixture was stirred at room temperature for 24 h and was filtered through celite. The filter cake was washed with ethyl acetate and the filtrate was concentrated to give compound 61 (0.67 g, 84%). ¹H NMR (CDCl₃, 400 MHz): δ 0.18 (s, 6H), 1.00 (s, 9H), 3.85 (s, 3H), 6.60 (dd, J=15.6, 7.6 Hz, 1H), 6.88 (d, J=7.2 Hz, 1H), 7.05-7.08 (m, 2H), 7.40 (d, J=16.0 Hz, 1H), 9.65 (d, J=7.2 Hz, 1H).

### (E)-3-(4-hydroxy-3-methoxyphenyl)acrylaldehyde (62)

Into a stirring solution of compound 7 (0.67 g, 2.29 mmol) in THF (10 mL) at 0° C., TBAF solution (1 M in THF, 2.52 mL, 2.52 mmol) was added dropwise. The mixture was stirred for 1 h and was quenched with a saturated solution of NaHCO₃ (stirred for 30 min). The organic layer was separated and the aqueous layer was extracted with DCM (3×15 mL). The organic layer was dried over Na₂SO₄ and concentrated. The crude product was purified by column chromatography (35% ethyl acetate in Hexane) to give compound 62 (0.343 g, 84%). It was immediately taken to the next step.

### (E)-4-(3-((2-amino-4,5,6,7-tetrahydrobenzo[d]thiazol-6-yl)(propyl)amino)prop-1-en-1-yl)-2-methoxyphenol (63)

A mixture of aldehyde 62 (0.334 g, 1.87 mmol) and (+) pramipexole (0.395 g, 1.87 mmol) in DCM (10 mL) was stirred for 1 h, and followed by the portionwise addition of NaBH(OAc)₃ (0.710 g, 3.35 mmol). Then the mixture was kept for stirring for 48 h at room temperature and was quenched with a saturated solution of NaHCO₃ afterwards at 0° C. The organic layer was separated and the aqueous layer was extracted with ethyl acetate (3×10 mL). The combined organic phase was dried over Na₂SO₄. The concentrated crude product was purified by column chromatography (15% methanol in DCM) to give compound 63 (0.21 g, 30%). ¹H NMR (CD₃OD, 400 MHz): δ 1.03 (t, J=7.2 Hz, 3H), 1.72 (q, J=7.1 Hz, 2H), 1.81-2.00 (m, 2H), 2.23-2.33 (m, 1H), 2.58-2.67 (m, 1H), 2.79-2.90 (m, 3H), 2.99 (t, J=7.1 Hz, 2H), 3.25 (dd, J=15.6, 5.2 Hz, 1H), 3.46 (s, 1H), 3.89 (s, 3H), 6.81 (d, J=8.4 Hz, 1H), 6.92 (dd, J=15.2, 7.6 Hz, 1H), 7.10 (d, J=8.0 Hz, 1H), 7.20 (s, 1H), 7.36 (d, J=16.4 Hz, 1H). The free base was converted into its corresponding hydrochloride salt. Mp 205-207° C. Anal. (C₂₀H₃₂Cl₃N₃O₃S) C, H, N.

| | Calculated | | | Found | | |
|---|---|---|---|---|---|---|
| Compound | C | H | N | C | H | N |
| 9. 3HCl•1H₂O | 47.96 | 6.44 | 8.39 | 48.24 | 6.35 | 8.29 |

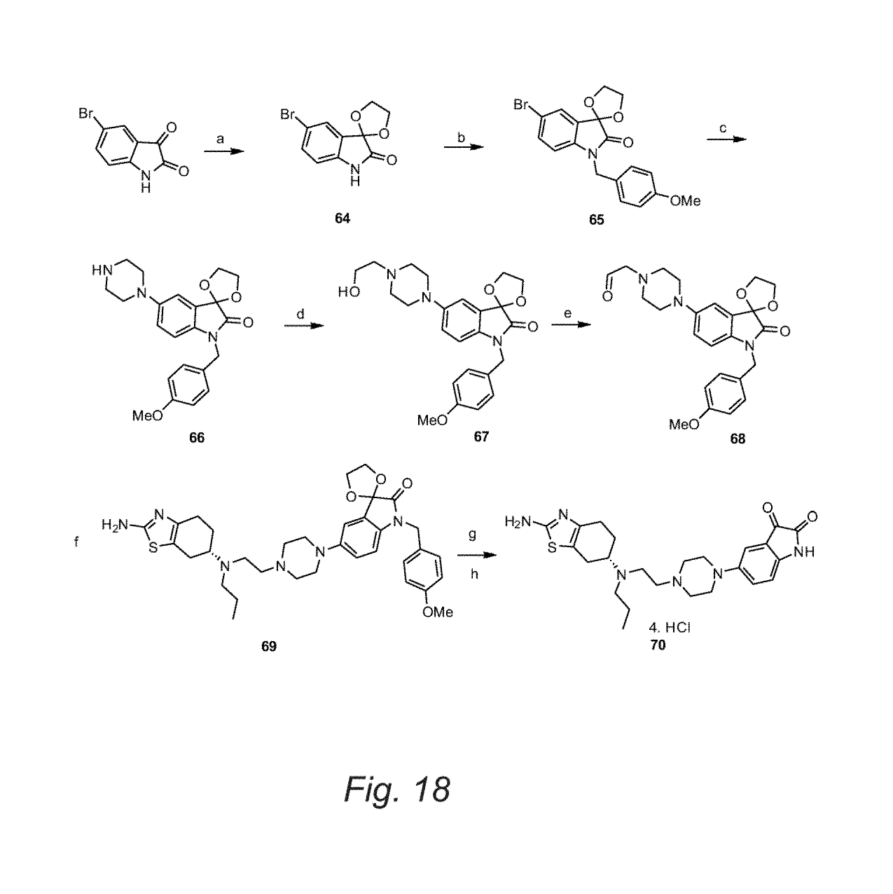

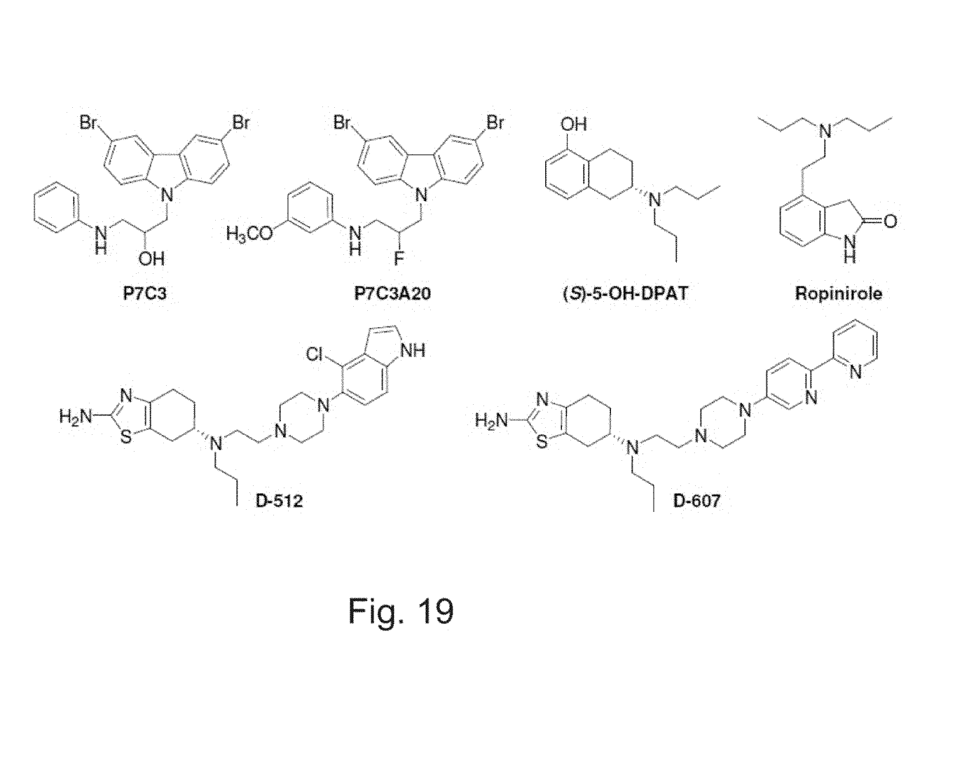

FIG. 18 provides a synthetic scheme for the compounds having formula I, specifically for compound 70.

Reagent and condition: (a) Ethylene glycol, Toluene, PTSA, reflux, 12 h; (b) 4-Methoxybenzylbromide, K₂CO₃, DMF, TBAI, rt, 12 h; (c) Piperazine, Pd(OAc)₂, BINAP, Cs₂CO₃, Toluene, reflux; (d) Bromoethanol, K₂CO₃, CH₃CN, reflux, 12 h; (e) (COCl)₂, DMSO, Et₃N, CH₂Cl₂, −78° C. to rt; (f) (+) (−) Pramipexole, Na(OAc)₃BH, CH₂Cl₂, 48 h; (g) 1:1 Conc. HCl, MeOH, sealed tube reflux, 12 h; (h) 2M HCl in ether, CH₂Cl₂.

### 5′-bromospiro[[1,3]dioxolane-2,3′-indolin]-2′-one (64)

To a solution of 5-bromoisatin (1 g, 4.42 mmol) in toluene (40 mL), ethylene glycol (4.9 mL, 84.48 mmol) and p-toluenesulphonic acid (38.1 mg, 0.22 mmol) were added. The reaction mixture was refluxed for 5 h and then evaporated to dryness. The residue was diluted with dichlomethane and washed with saturated sodium bicarbonate solution. The aqueous layer was extracted with dichloromethane three times. The combined organic extracts were dried over anhydrous sodium sulfate, filtered, and evaporated. The crude mixture was purified using column chromatography to yield 64. Yield: 99%. (C. H. Wang, A. R. White, S. N. Schwartz, S. Alluri, T. M. Cattabiani, L. K. Zhang, T. M. Chan, A. V. Buevich, A. K. Ganguly Tetrahedron 2012, 68, 9750-9762.)

### 5′-bromo-1′-(4-methoxybenzyl)spiro[[1,3]dioxolane-2,3′-indolin]-2′-one (65)

A mixture of compound 55 (1.75 g, 6.38 mmol), (4-Methoxybenzylbromide) (1.3 mL, 9.5 mmol), pinch of tetrabutylammonium iodide and K₂CO₃ (2.64 μg, 19.1 mmol) in DMF (15 mL) was stirred for 12 h. Quench the reaction mixture with ice cold water and extract with ethylacetate, dried over sodium sulfate and evaporated the crude material was purified by silica gel column chromatography (EtOAc/hexane, 3:1) to give compound 65 (2.25 g, 90%)¹H NMR (400 MHz, CDCl₃): δ 7.47 (d, J=1.6 Hz, 1H), 7.35 (dd, J₁=8.4 Hz, J₂=1.6 Hz, 1H), 7.18 (d, J=8.8 Hz, 2H), 7.35 (dd, J₁=8.4 Hz, J₂=1.6 Hz, 1H), 6.54 (d, J=8.0 Hz, 1H), 4.74 (s, 2H), 4.62-4.59 (m, 2H), 4.35-4.32 (m, 2H), 3.77 (s, 3H); ); ¹³C NMR (100 MHz, CDCl₃): δ 172.8, 159.2, 142.8, 134.2, 128.5 (2C), 128.1, 126.7, 115.8, 114.3 (2C), 111.2, 66.0, 55.2, 43.0.

### 1′-(4-methoxybenzyl)-5′-(piperazin-1-yl)spiro[[1,3]dioxolane-2,3′-indolin]-2′-one (66)

A mixture of 2 (1.2 g, 3.0 mmol), Piparazine (0.78 g, 9.1 mmol), Pd(OAc)₂ (0.034 g, 0.15 mmol), BINAP (0.142 g, 0.22 mmol) and Cs₂CO₃ (2.96 g, 9.2 mmol) in toluene (12 mL) was heated at 110° C. for 12 h. The reaction mixture was filtered through Celite, wash with dicholoromethane and concentrated in vacuo. The crude residue was dissolved in ethylacetate and washed with water and evaporated to afford compound 66 (0.755 g, 62%). The crude residue was used for next step without purification. ¹H NMR (400 MHz, CDCl₃): δ 7.19 (d, J=8.4 Hz, 2H), 7.01 (d, J=2.4 Hz, 1H), 6.81 (d, J=8.4 Hz, 2H), 6.77 (dd, J₁=8 Hz, J₂=2.4 Hz, 1H), 6.55 (d, J=8.8 Hz, 1H), 4.70 (s, 2H), 4.63-4.60 (m, 2H), 4.33-4.30 (m, 2H), 3.75 (s, 3H), 3.01 (d, J=5.6 Hz, 8H); 13C NMR (100 MHz, CDCl₃): δ 173.2, 159.0, 148.7, 136.8, 128.5 (2C), 127.4, 124.6, 119.4, 114.5 (2C), 110.1, 102.5, 65.8, 55.2, 51.1 (2C), 45.8 (2C), 42.9.

### 5′-(4-(2-hydroxyethyl)piperazin-1-yl)-1′-(4-methoxybenzyl)spiro[[1,3]dioxolane-2,3′-indolin]-2′-one (67)

A mixture of compound 57 (0.74 g, 1.86 mmol), (2-bromoethanol (0.26 mL, 3.7 mmol), and K₂CO₃ (0.76 g, 5.55 mmol) in CH₃CN (15 mL) was refluxed for 12 h. After filtration, acetonitrile was evaporated under reduced pressure and the crude material was purified by silica gel column chromatography (EtOAc/hexane, 3:1) to give compound 67 (0.631 g, 77%). ¹H NMR (400 MHz, CDCl₃): δ 7.19 (d, J=9.2 Hz, 2H), 7.01 (d, J=2.4 Hz, 1H), 6.82 (d, J=9.2 Hz, 2H), 6.77 (dd, J₁=8 Hz, J₂=2.4 Hz, 1H), 6.56 (d, J=8 Hz, 1H), 4.71 (s, 2H), 4.63-4.60 (m, 2H), 4.34-4.31 (m, 2H), 3.75 (s, 3H), 3.76 (t, J=5.6 Hz, 2H), 3.53 (bs, 1H), 3.11 (t, J=4.8 Hz, 4H), 2.69 (t, J=4.8 Hz, 4H), 2.62 (t, J=5.6 Hz, 2H); 13C NMR (100 MHz, CDCl₃): δ 173.2, 159.0, 148.0, 136.9, 128.5 (2C), 127.3, 124.7, 119.3, 114.5, 114.1 (2C), 110.2, 102.5, 65.8 (2C), 59.3, 57.6, 55.2, 52.8 (2C), 49.9 (2C), 42.9.

### 2-(4-(1′-(4-methoxybenzyl)-2′-oxospiro[[1,3]dioxolane-2,3′-indolin]-5′-yl)piperazin-1-yl)acetaldehyde (68)

Into a stirring solution of oxalyl chloride (0.094 mL, 1.1 mmol) in CH₂Cl₂ (7 mL) at −78° C. was added DMSO (0.141 mL, 1.99 mmol). The reaction mixture was stirred for 0.5 h, followed by addition of compound 67 (0.25 g, 0.55 mmol, in 5 mL of CH₂Cl₂. The reaction mixture was stirred at the same temperature for 0.5 h, followed by addition of Et₃N (0.615 mL, 4.4 mmol), and stirring was continued for 1.5 h while allowing the reaction mixture to reach room temperature. The reaction mixture was quenched by addition of a saturated solution of NaHCO₃ and extracted with CH₂Cl₂ (3×100 mL). The combined organic layer was dried using Na₂SO₄, and the solvent was removed under reduced pressure. The crude product was purified by silica gel column chromatography (EtOAc/MeOH, 20:1) to give compound 68 (0.24 g, 99%). ¹H NMR (600 MHz, CDCl₃): δ 9.71 (d, J=1.2 Hz, 1H), 7.19 (d, J=9.0 Hz, 2H), 7.01 (d, J=2.4 Hz, 1H), 6.81 (dd, J₁=6.6 Hz, J₂=1.8 Hz, 2H), 6.77 (dd, J₁=8.4 Hz, J₂=2.4 Hz, 1H), 6.55 (d, J=8.4 Hz, 1H), 4.71 (s, 2H), 4.63-4.61 (m, 2H), 4.33-4.31 (m, 2H), 3.75 (s, 3H), 3.22 (d, J=1.2 Hz, 2H), 3.13 (t, J=4.8 Hz, 4H), 2.65 (t, J=4.8 Hz, 4H).

### 5′-(4-(2-((2-amino-4,5,6,7-tetrahydrobenzo[d]thiazol-6-yl)(propyl)amino)ethyl)piperazin-1-yl)-1′-(4-methoxybenzyl)spiro[[1,3]dioxolane-2,3′-indolin]-2′-one (69)

Into a stirring solution of Pramipexole (0.112 g, 5.3 mmol) in CH₂Cl₂ (50 mL) was added aldehyde 68 (0.24 g, 5.3 mmol). After the mixture was stirred for 1 h, NaBH(OAc)₃ (0.23 g, 1.1 mmol) was added portion wise and the mixture was stirred for 48 h at room temperature. The reaction mixture was quenched with a saturated solution of NaHCO₃ at 0° C. and extracted with dicholoromethane (3×100 mL). The combined organic layer was dried over Na₂SO₄, and the solvent was removed under reduced pressure. Crude product was purified by column chromatography (EtOAc/MeOH, 20:1) to give compound 69 (0.234 g, 66%). ¹H NMR (400 MHz, CDCl₃): δ 7.20 (d, J=8.4 Hz, 2H), 7.01 (d, J=2.4 Hz, 1H), 6.82 (d, J=8 Hz, 2H), 6.76 (dd, J₁=8 Hz, J₂=2.4 Hz, 1H), 6.55 (d, J=8.0 Hz, 1H), 4.81 (bs, —NH₂, 2H), 4.71 (s, 2H), 4.62 (t, J₁=6.4 Hz, 2H), 4.32 (t, J₁=6.8 Hz, 2H), 3.75 (s, 3H), 3.08-3.06 (m, 5H), 2.69-2.44 (m, 14H), 1.99-1.96 (m, 1H), 1.72-1.68 (m, 1H), 1.48-1.43 (m, 2H), 0.87 (t, J=7.2 Hz, 3H); ¹³C NMR (100 MHz, CDCl₃): δ 173.2, 165.5, 159.0, 148.2, 144.9, 136.6, 128.5, 127.4, 124.6, 119.0, 117.3, 114.2, 114.1, 110.1, 102.6, 65.8, 58.5, 58.0, 55.2, 53.6, 53.5, 49.9, 48.3, 42.9, 26.5, 25.7, 25.0, 22.2, 11.8.

### (S)-5-(4-(4-(2-((2-amino-4,5,6,7-tetrahydrobenzo[d]thiazol-6-yl)(propyl)amino)ethyl)piperazin-1-yl)phenyl)indoline-2,3-dione (70)

To a solution of 69 (0.05 g, 0.079 mmol) in 1:1 mixture of Conc. HCl and methanol (6 mL) was taken in a sealed tube. After that, the mixture was heated to 110° C. for 12 h. The reaction mixture was quenched with a saturated solution of NaHCO₃ at 0° C. and extracted with dicholoromethane (3×100 mL). The combined organic layer was dried over Na₂SO₄, and the solvent was removed under reduced pressure. Crude product was purified by column chromatography (CH₂Cl₂/MeOH, 20:1) to give compound 70 (0.03 g, 69%). The pure amine was dissolved in 20:1 mixture of CH₂Cl₂:MeOH (0.5 mL), then added 2 M HCl (5 mL) at room temperature and stirred for 10 min. Evaporate the solvent using N₂ and then wash with ether followed by vaccume dry to form HCl salt of 70. Free amine: ¹H NMR (400 MHz, CDCl₃): δ 8.56 (s, —NH, 1H), 7.16-7.12 (m, 2H), 6.80 (d, J=8.8 Hz, 1H), 4.99 (bs, —NH₂, 2H), 3.13-3.10 (m, 4H), 3.04-3.01 (m, 1H), 2.72-2.44 (m, 14H), 1.99-1.96 (m, 1H), 1.73-1.69 (m, 1H), 1.49-1.43 (m, 2H), 0.88 (t, J=7.2 Hz, 3H); ¹³C NMR (100 MHz, CDCl₃): δ 183.7, 165.9, 148.5, 144.7, 142.5, 135.4, 126.6, 118.5, 117.1, 113.0, 112.7, 58.5, 58.0, 53.46, 49.6, 48.3, 41.3, 29.6, 26.4, 25.8, 25.0, 22.3, 11.8. LRMS (ESI) m/z [M+H]+: calcd. for C₂₄H₃₃N₆O₂S: 469.2386, found: 469.4826. The product was converted into corresponding hydrochloride salt. Mp: 312-314° C.; Anal. (C₂₄H₃₂N₆O₂S.4HCl.2H₂O.

The following table provides inhibition constants for displacing [3H]spiperone binding to the cloned D2L and D₃ receptors expressed in HEK cells. Results are means±SEM for 3 experiments each performed in triplicate. NR=Not repeated.

TABLE 2
Inhibition constants

| | Ki, (nM), D2L | Ki, (nM), D3 |
|---|---|---|
| Compound | [³H]Spiperone | [³H]Spiperone |
| 22 (D-547) | 2,404 ± 29 | 15.8 ± 2.0 |
| 23 (D-548) | 175 ± 8 | 3.62 (4) ± 0.78 |
| (−)-23 (D-591) | 68.3 ± 12.0 | 1.57 ± 0.41 |
| 34 (D-575) | 116 ± 8 | 2.59 ± 0.31 |
| (−)-34 (D-593) | 65.7 ± 11.7 | 1.42 ± 0.14 |
| 35 (D-584) | 117 ± 17 | 1.01 ± 0.28 |
| (−)-36 (D-601) | 112 ± 8 | 0.78 ± 0.2 |
| 39 (D-567) | 233 ± 8 | 23.3 ± 4.0 |
| 46, 47 (D-592) | 121 ± 21 | 25.0 ± 1.96 |
| 54 (D-573) | 499 ± 16 | 11.9 ± 1.9 |
| 63 (D-570) | 4,634 ± 325 | 39.1 ± 5.4 |
| 70 (D-588) | 162 ± 7 | 14.2 ± 3.8 |

Compounds Having Carbazole Functionality

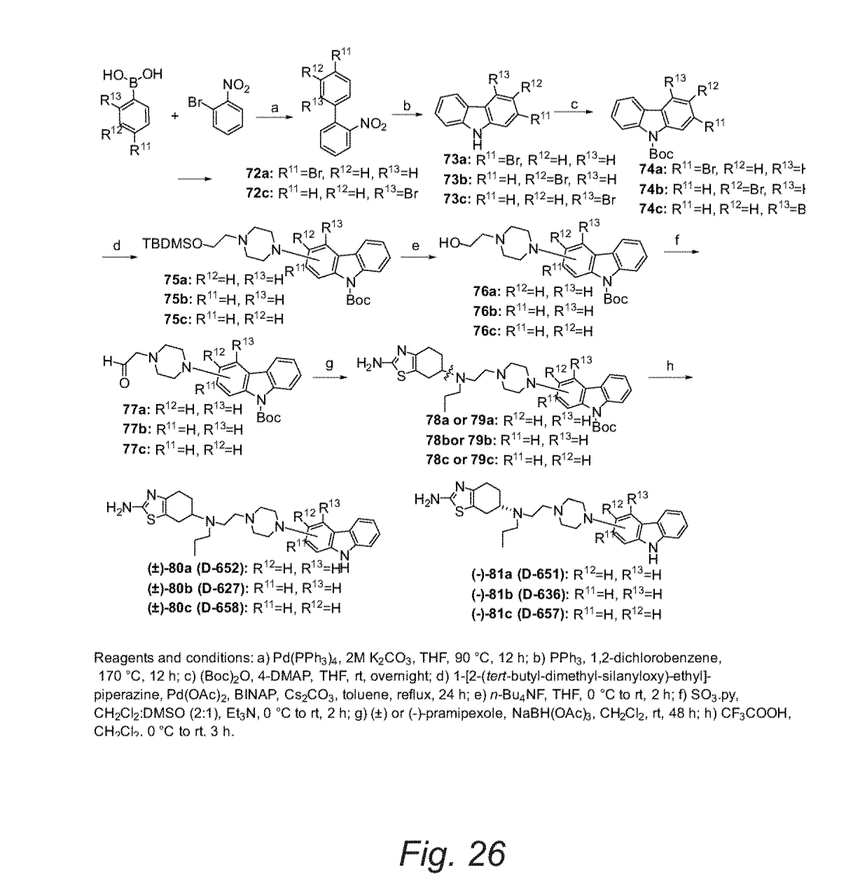

A series of compounds were synthesized by incorporating the aminotetralin or bioisosteric equivalent agonist head group with carbazole functionality via ethylpiperazine linker (schemes of FIGS. 26, 27, 28). The scheme of FIG. 26 describes the syntheses of final compounds (+)-80a, (+)-80b, (+)-80c and (−)-81a, (−)-81b, (−)-81c. A palladium catalyzed coupling of (4-bromophenyl)boronic acid and 1-bromo-2-nitrobenzene afforded 4′-bromo-2-nitro-1,1′-biphenyl (72a). This compound was then cyclized in the presence of PPh₃ to produce 2-bromo-9H-carbazole (73a) which then underwent N-protection using di-tert-butyl dicarbonate in the presence of 4-dimethylaminopyridine (4-DMAP) to yield 74a. Palladium-catalyzed mediated cross coupling of 74a with 1-(2-((tert-butyldimethylsilyl)oxy)-ethyl)piperazine under refluxing conditions in the presence of Cs₂CO₃ and BINAP in toluene produced intermediate 75a. Treatment with n-Bu₄NF (TBAF) in THF removed the silyl protecting group of compound 75a to produce the alcohol 76a, which in the presence of pyridine-sulfur trioxide was oxidized to yield the corresponding aldehyde 77a. Reductive amination of the aldehyde with either (±) or (−)-pramipexole in the presence of NaBH(OAc)₃ afforded compounds 78a and 79a, respectively. In the final step, the amine protecting t-Boc groups were removed by treatment with trifluoroacetic acid to produce the final compounds (+)-80a and (−)-81a as TFA salts. The other final compounds (+)-80b, (+)-80c, (−)-81b and (−)-81c were also synthesized in the similar fashion as described above, where 3-Bromo-9H-carbazole and 4-Bromo-9H-carbazole were used as the starting materials, respectively.

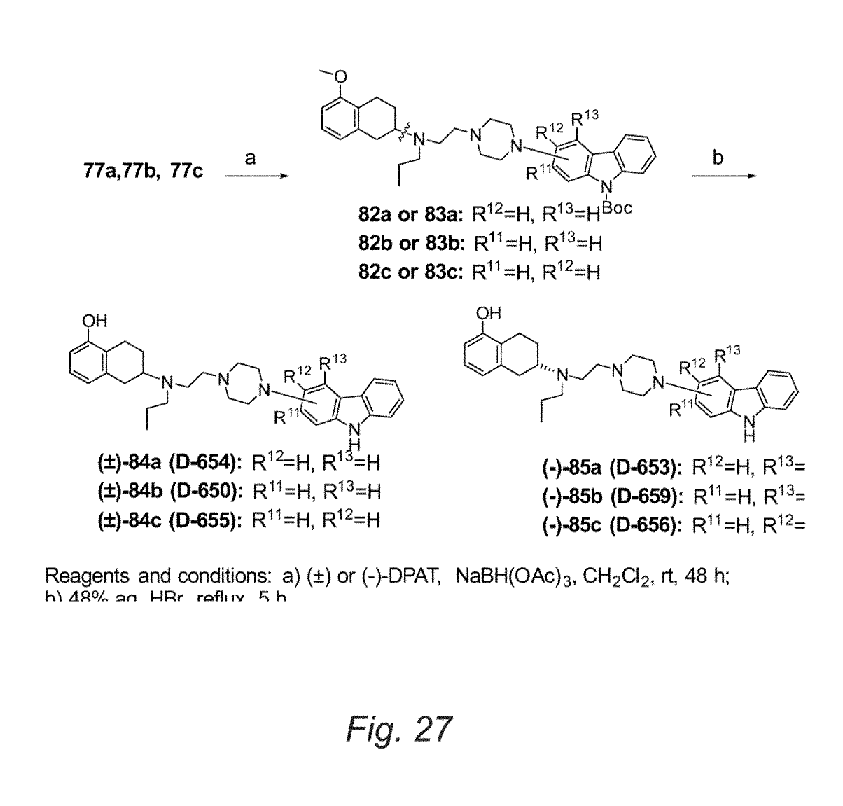

The syntheses of the final compounds (±)-84a, (±)-84b, (±)-84c and (−)-85a, (−)-85b, (−)-85c is shown in the scheme of FIG. 27. To prepare these compounds, we employed (I) and (−)-(5-methoxy-1,2,3,4-tetrahydro-naphthalen-2-yl)-propyl-amine and reductively alkylated with intermediate aldehydes 77a-77c in the presence of NaBH(OAc)₃ to produce compounds (±)-82a, (±)-82b, (±)-82c, (−)-83a, (−)-83b, and (−)-83c. Finally, demethylation and removal of the protecting t-Boc groups were carried out in one step by refluxing with aq. HBr to give the final compounds (±)-84a, (±)-84b, (±)-84c, (−)-85a, (−)-85b, and (−)-85c as HBr salts.

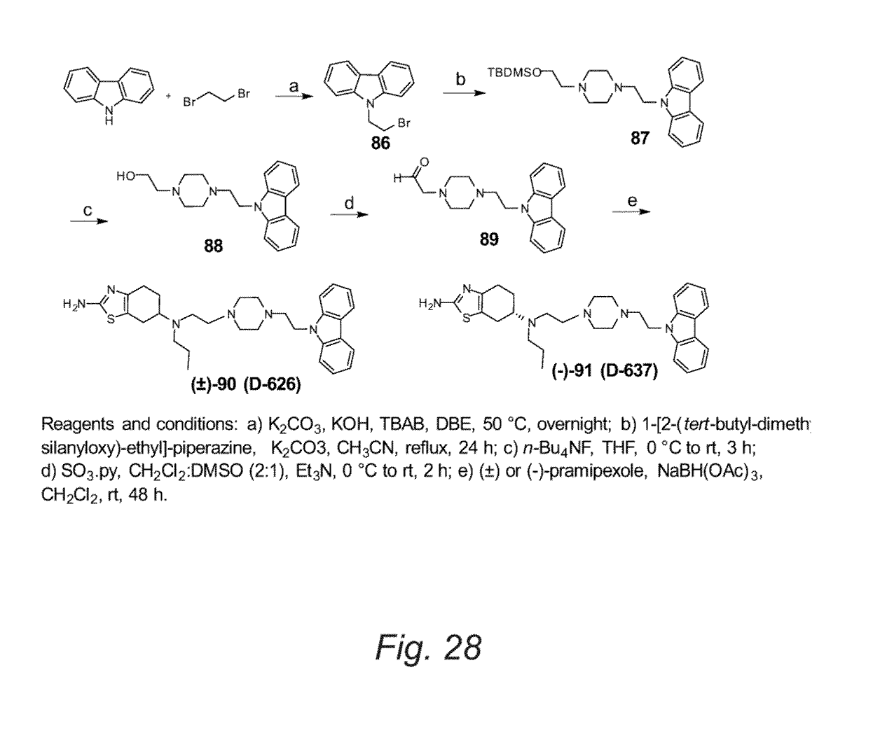

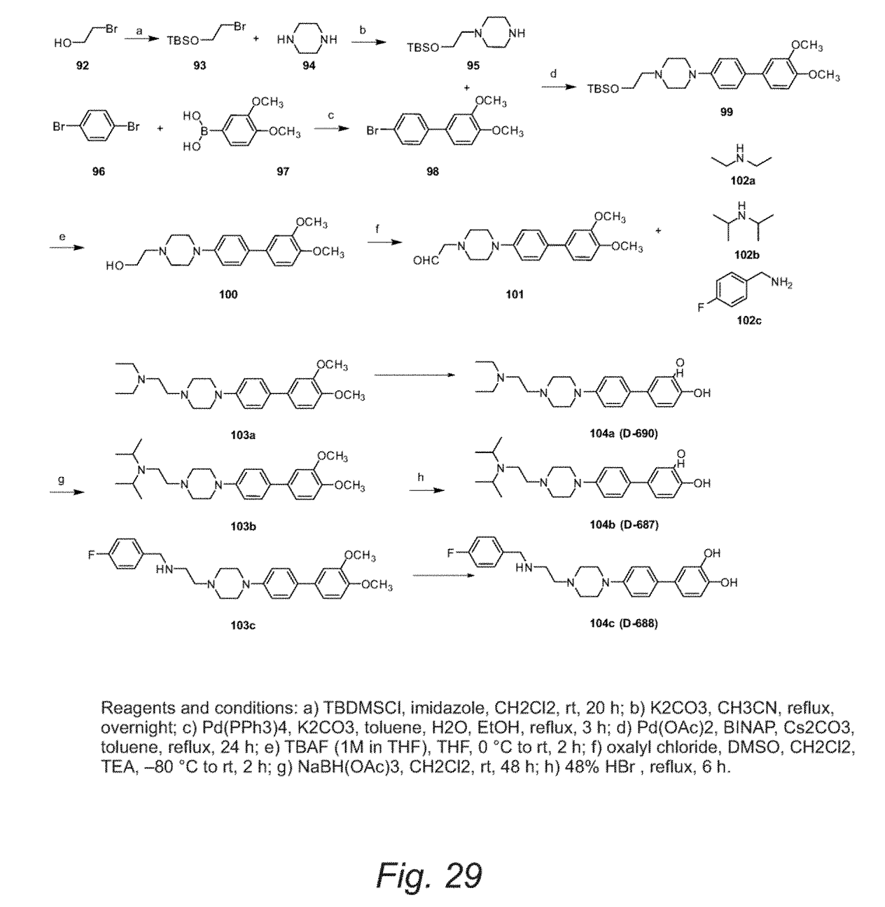

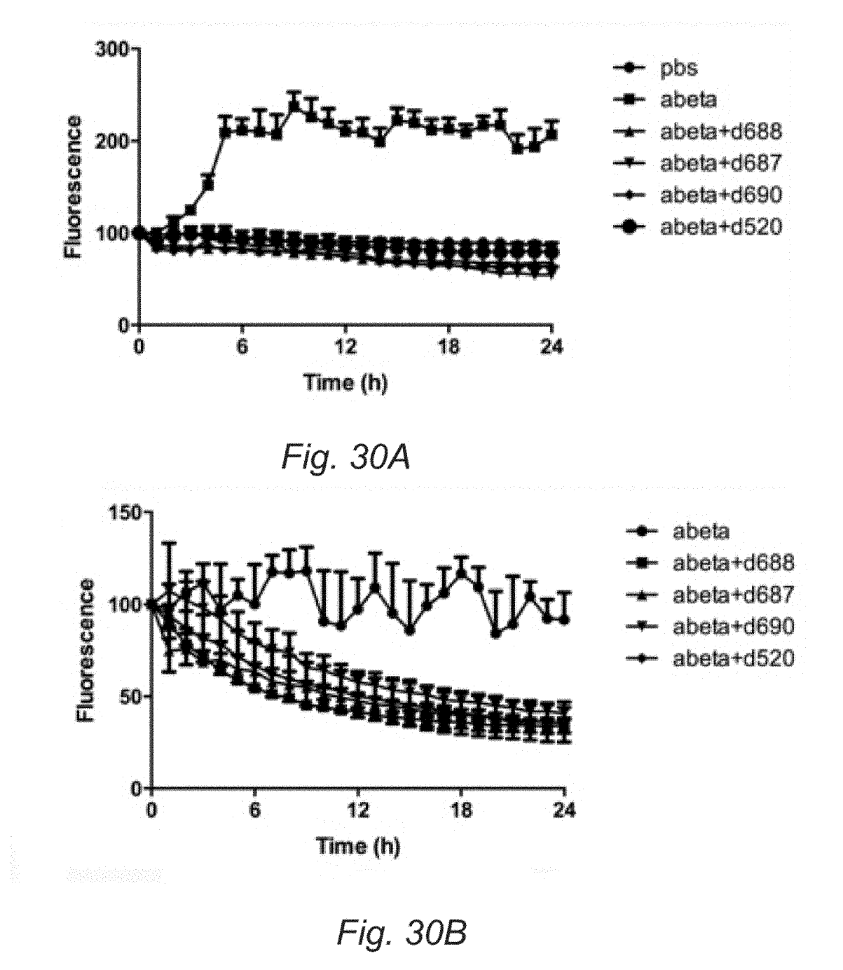

The syntheses of two more target compounds (+)-90 and (−)-91 are illustrated in the scheme of FIG. 28. N-alkylation was first performed by refluxing carbazole with dibromoethane in presence of a mixture of K₂CO₃, KOH and TBAB to afford 9-(2-bromoethyl)-9H-carbazole 86. Base-catalyzed condensation of 1-(2-((tert-butyldimethylsilyl)-oxy)ethyl)piperazine with intermediate 86 yielded compound 87, which on TBDMS deprotection in the presence of TBAF in THF afforded alcohol 88 in excellent yield. Pyridine-sulfur trioxide was used to oxidize alcohol 88 next to yield the corresponding aldehyde 99, which was then underwent reductive amination with either (±) or (−)-pramipexole in the presence of NaBH(OAc)₃ to produce the final compounds (±)-90 and (−)-91. These two molecules were converted to their corresponding HCl salts by treatment with ethereal HCl. All the final compounds were characterized by ¹H and ¹³C NMR as well as elemental analysis.

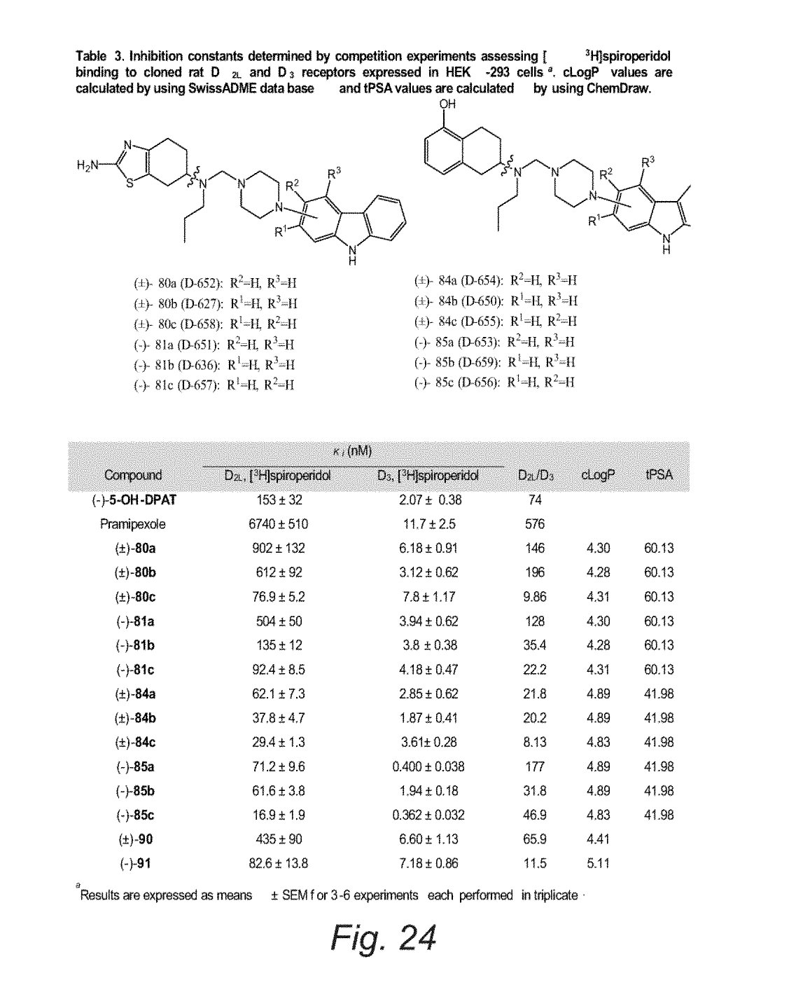

A well established radioligand competition assay was carried out to evaluate binding affinity of the test compounds and were compared with that of the reference agent (S)-5-OH-DPAT (FIG. 24, Table 3). Binding affinity to rat DA D₂ and D₃ receptors expressed in HEK-293 cells was determined as described by us previously. Table 3 lists the binding data of new compounds. Compounds (±)-80a-c, which incorporate racemic 2-aminothiazole head group and a piperazine ring connected to the different positions of the carbazole ring, exhibited high affinity for D₃ and low to moderate affinity for D₂ receptors. When the positions of attachment are at carbon 2 and 3 of the carbazole moiety for compounds 80a and 80b, respectively, both the compounds displayed low affinity for D₂ and high affinity for the D₃ receptors with high selectivity (Kᵢ, D₂=902 nM, D₃=6.18 nM, D₂/D₃=146 and D₂=612 nM, D₃=3.12 nM, D₂/D₃=196 for 80a and 80b, respectively). Interestingly, covalent attachment at position 4 of the carbazole ring dramatically improved the affinity for D₂ while that for D₃ receptor remained the same (Kᵢ, D₂=76.9 nM, D₃=7.8 nM, D₂/D₃=9.86 for 80c). This indicated highest tolerance of the 4-substituted carbazole derivative for interaction with the D2 and D3 receptors. As expected, we observed a 2-4-fold improvement in binding affinity when enantiomerically pure aminothiazole moiety was attached to the carbazole as in (−)-81a and (−)-81b compared to their racemic counterparts (Kᵢ, D₂=504 nM, D₃=3.94 nM, D₂/D₃=128 and D₂=135 nM, D₃=3.80 nM, D₂/D₃=35 for (−)-81a and (−)-81b respectively). However, for (−)-81c we did not observe much difference from its racemic version.

Next, the effect of bioisosteric replacement of the aminothiazole moiety with aminotetraline functionality on the receptor binding of target compounds was evaluated. In corroboration with our previous results, aminotetraline substituted compounds (+)-84a-c and (−)-85a-c exhibited high affinity at both D₂ and D₃ receptors. For instance, the aminotetraline analogue (−)-85a has been found to have very high affinity for D₂ while displaying subnanomolar affinity for D₃ receptor compared to the corresponding thiazolidium counterpart (−)-81a (Kᵢ, D₂=71.2 nM, D₃=0.40 nM, D₂/D₃=177 for (−)-85a vs D₂=504 nM, D₃=3.94 nM, D₂/D₃=128 for (−)-81a). Among the three enantiomerically pure isomers (−)-85a-c, which differ only in the substitution positions at the carbazole moiety, positions 2, 3 and 4, showed variable binding affinity at both D₂ and D₃ receptors (Kᵢ, D₂=71.2 nM, D₃=0.40 nM for (−)-85a (; D₂=61.6 nM, D₃=1.94 nM for (−)-85b and D₂=16.9 nM, D₃=0.36 nM for (−)-85c). As discussed before, substitution at the 4-position of the carbazole aromatic ring resulted in compounds 80c, (−)-81c, 84c and (−)-85c) with better D₂/D₃ binding affinities in comparison to other isomeric analogues with compound (−)-85c exhibiting the highest affinity among all the molecules, underscoring the importance of positional attachment to the carbazole ring. Finally, the binding affinities were evaluated for another series of compounds in which the piperazine ring of the agonist fragment was appended directly to the carbazole nitrogen atom through a methylene linker. As shown in Table 3, enantiomeric compound (−)-91 displayed relatively higher binding affinity at D₂ and comparable affinity at D₃ receptor with moderate selectivity compared to the racemic compound (±)-90 (Kᵢ, D₂=435 nM, D₃=6.60 nM, D₂/D₃=65.9 and D₂=82.6 nM, D₃=7.18 nM, D₂/D₃=12 for 90 and (−)-91, respectively). This structural modification suggests no significant differences in DA receptor interaction between compounds where the carbazole moiety is attached either at the ⅔ positions of the aromatic ring or through the nitrogen atom; however, a prominent difference exists for compounds where the carbazole nitrogen is sterically free to probably participate in additional receptor interaction (e.g. (−)-85c vs (−)-85b and (−)-91).

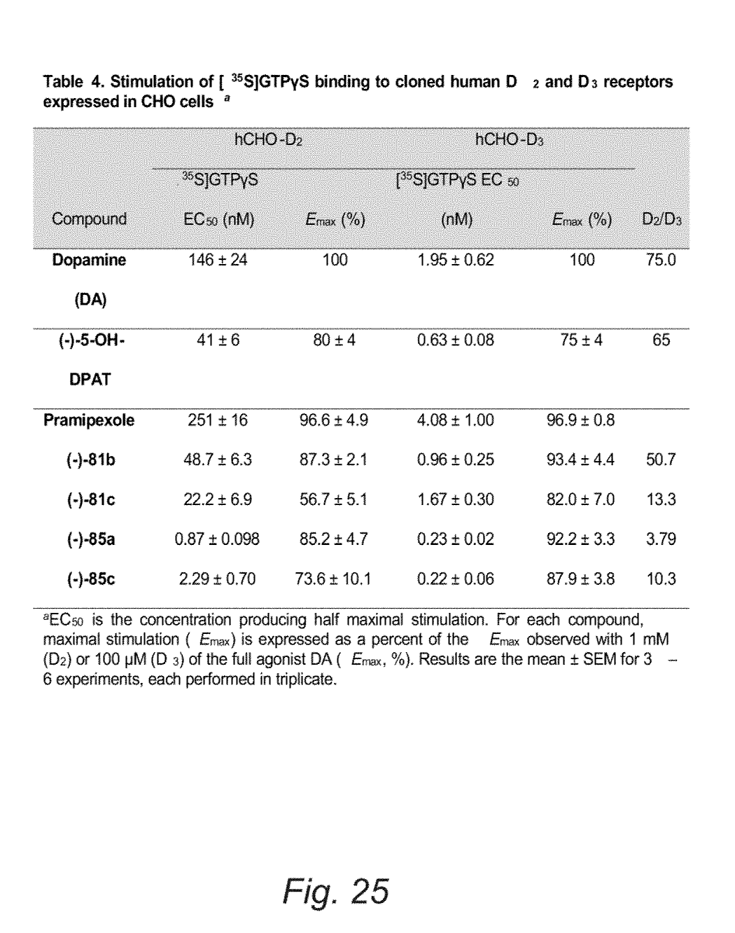

Based on binding data, functional activities of the selected optically active lead compounds for human DA D₂ and D₃ receptors expressed in CHO cells were measured by using [³⁵S]GTPγS binding assay. Agonist or partial agonist activity was judged by comparing with the maximum stimulation (Eₘₐₓ), produced by the full agonist DA. As shown in Table 4 (FIG. 25), aminothiazole containing compounds (−)-81b and (−)-81c demonstrated moderate potency at both D₂ and D₃ receptors (EC₅₀ (GTPγS); D₂=48.7, D₃=0.96 nM and D₂=22.2, D₃=1.67 nM, respectively), correlating well with binding data. While (−)-81b showed full agonist activity at both D₂ and D₃ receptors (Eₘₐₓ=87-93%), compound (−)-81c revealed partial agonist activity at D₂ and full agonism at D₃ receptor (Eₘₐₓ=57% vs 82%, respectively for D₂ and D₃). On the other hand, aminotetraline compound (−)-85a displayed very high functional potency (EC₅₀ (GTPγS); D₂=0.87 and D₃=0.23 nM) and full agonism (Eₘₐₓ=85-92%) at both the receptors. Compound (−)-85c was also found to be highly potent and efficacious in stimulating both receptors (EC₅₀ (GTPγS); D₂=2.29 and D₃=0.22 nM; Eₘₐₓ=74-88%). Neither compounds exhibited appreciable selectivity for D₃ over D₂ (Table 4) and their selectivity for D₃ receptor was much less when compared to the binding data (Table 3). The values for the ClogP and tPSA for all the compounds were calculated as shown in Table 3. In general, these values predict that these compounds should cross the blood brain barrier to produce in vivo CNS efficacy which we observed in case of (−)-81b, (−)-85a and (−)-85c (see below). Therefore, our SAR results of a series of carbazole compounds show that the affinity and selectivity for the D₂/D₃ receptors are governed by the nature of covalent attachment to the carbazole moiety and the structure of agonist binding head group in the hybrid molecule.

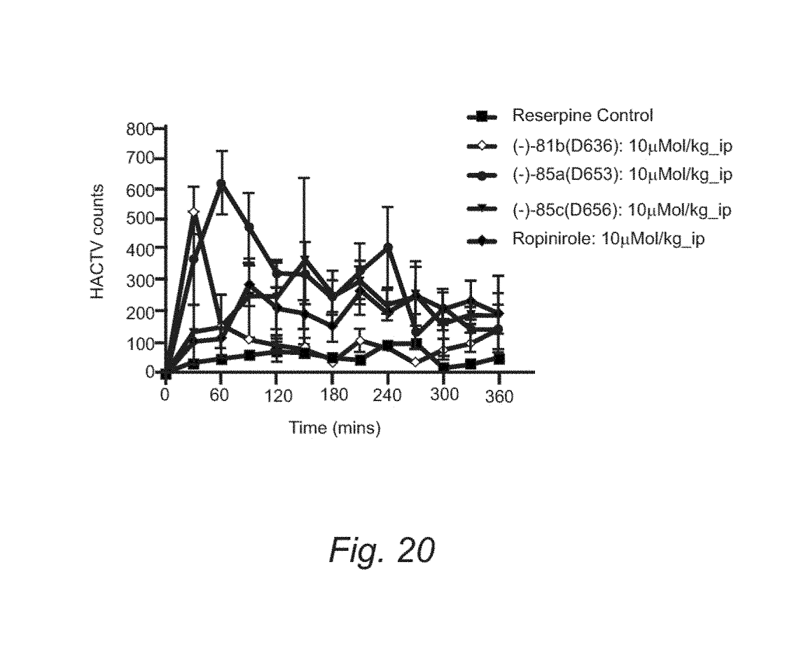

In vivo evaluation of the compounds (−)-81b, (−)-85a and (−)-85c in a reserpine-based PD animal model was performed next. In this model depletion of catecholamines by reserpine results in catalepsy in rats. This is a well-established animal model for PD. Reserpine produced a significant inhibition of locomotion in rats 18 h after the administration of the drug (5 mg/kg, sc), which indicated the development of akinesia (FIG. 20). Compounds (−)-81b, (−)-85a and (−)-85c at the dose of 10 μMol/kg, ip, significantly reversed akinesia in rats compared to reserprine treatment alone. The compounds also demonstrated significant enhancement of locomotion for the duration of the study of 6 h. Among the molecules tested, (−)-85a was found to have the highest in vivo activity and this finding correlates with in vitro functional assay where the compound exhibited subnanomolar potency for stimulation of D₂/D₃ receptors along with full agonist property. On the other hand, the reference drug ropinirole at 10 μMol/kg, ip, produced a fast onset of activation of locomotion compared to the control but had a shorter duration of action compared to the test compounds. It has been shown that the locomotor stimulation in this reserpine model is mediated by postsynaptic D₂/D₃ receptor activation by the compounds.

Antioxidant Activity

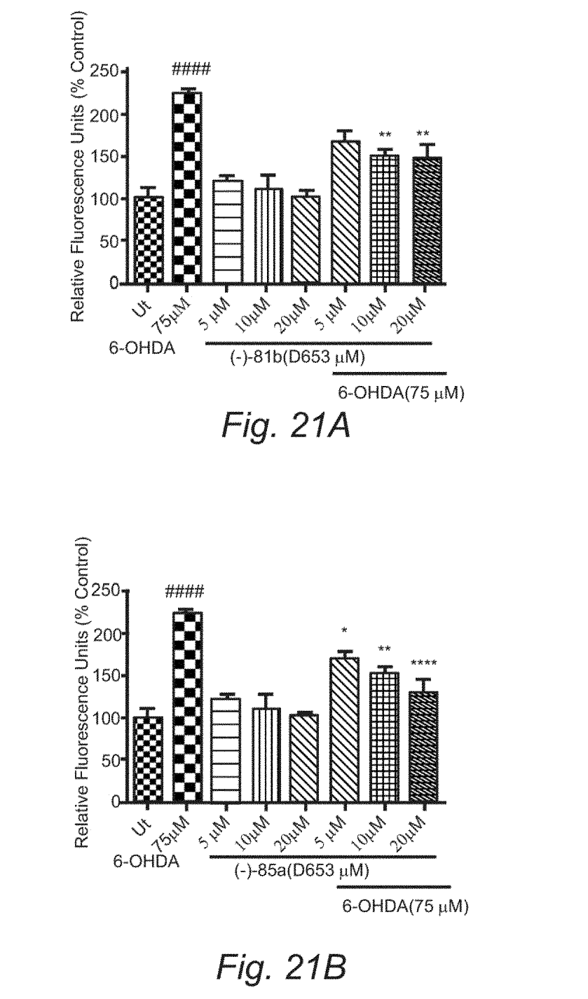

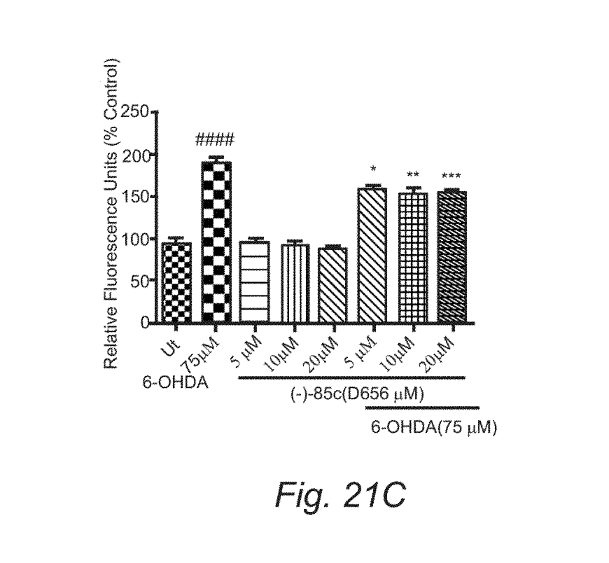

Experimental detection of reactive oxygen species (ROS) produced by 6-OHDA was carried out by DCFDA assay. 6-OHDA, a well known neurotoxin, produces oxidative stress similar to what is observed in PD and the mechanism of neurotoxicity of 6-OHDA takes place via its auto-oxidation and subsequent generation of hydrogen peroxide. DCFDA is a non-fluorogenic dye but in the presence of ROS, it is oxidized to produce DCF which produces florescence. We have previously shown that 6-OHDA causes cell death in a dose-dependent manner. We chose 75 μM 6-OHDA as we have shown that this dose induces 40-50% cell death. To examine whether our compounds (−)-81b, (−)-85a and (−)-85c can protect PC12 cells from the ROS produced by the exposure to 75 μM 6-OHDA, the PC12 cells were treated with 6-OHDA after pretreatment with various concentrations of drugs (5, 10, 20 μM) for 24 h, and compared with 6-OHDA alone treated cells. As shown in FIG. 21, a well over two-fold increase in ROS was observed in cells treated with 6-OHDA (75 μM) alone compared to the control untreated cells. However, the test compounds could dose dependently decrease the production of ROS induced by 6-OHDA (75 μM) in PC12 cells. In this regard, highest dose of all the three compounds was the most efficacious in producing significant antioxidant effect. Thus at 20 μM, a reduction in 76%, 93% and 36% ROS were induced by (−)-81b, (−)-85a and (−)-85c, respectively (FIG. 21A-C). Thus, (−)-85a was found to be the most potent antioxidant (FIG. 21B).

Cellular Neuroprotection Activity

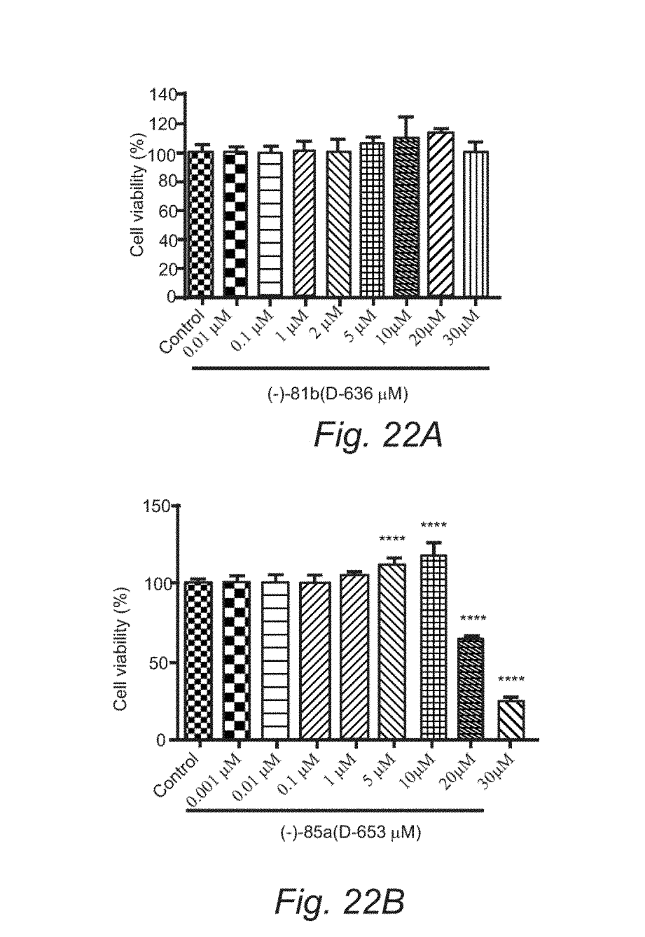

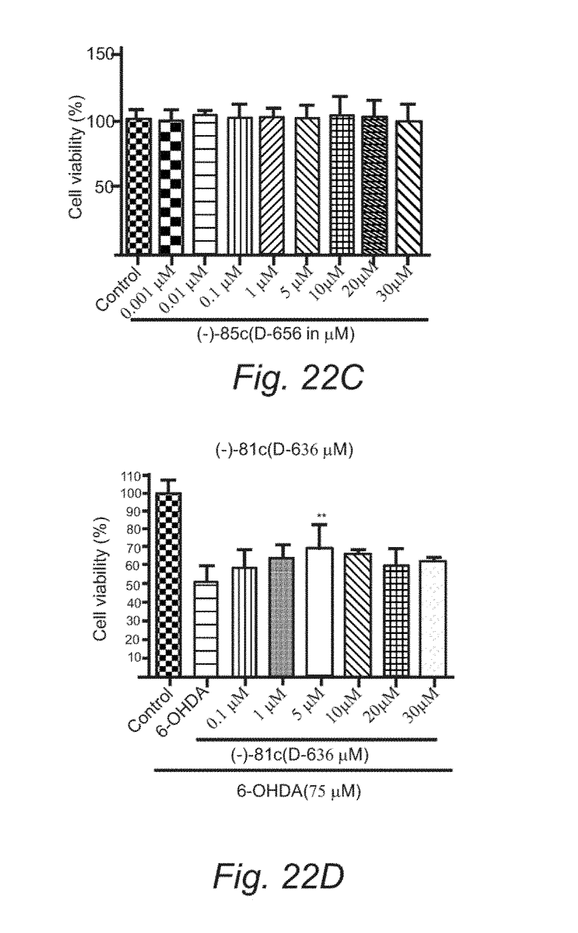

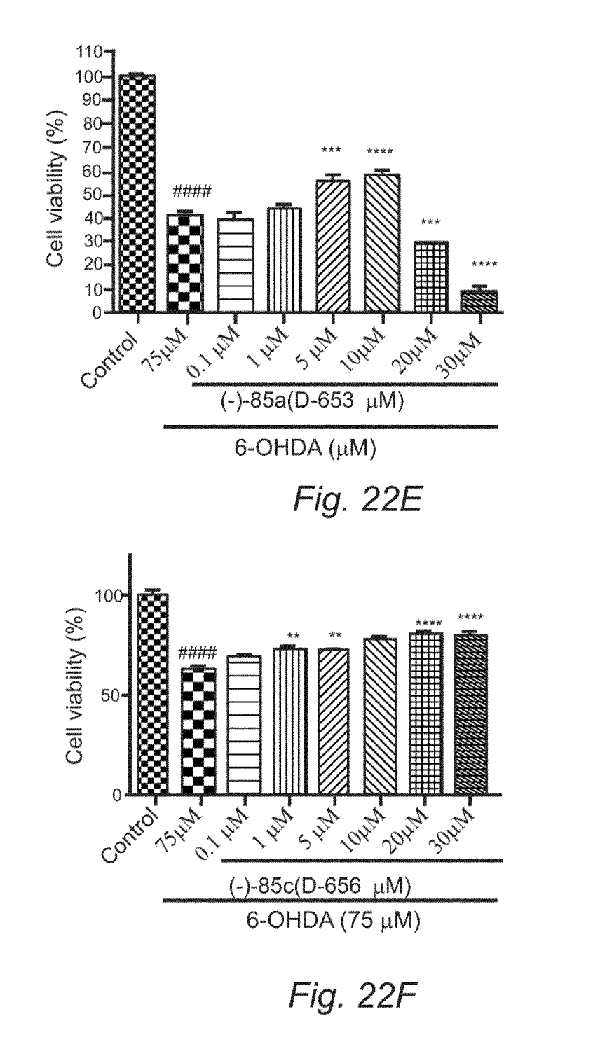

To investigate the neuroprotection property of the selected target molecules (−)-81b, (−)-85a and (−)-85c against 6-OHDA-induced cytotoxicity, we used dopaminergic rat adrenal Pheochromocytoma PC12 cells. As reported above, treatment of PC12 cells with 6-OHDA at 75 μM for 24 h produced ˜50% loss in cells viability and this concentration was used in subsequent experiments. In contrast, it was observed that the cells treated with increasing concentrations of either (−)-81b or (−)-85c alone (0.01-30 μM) showed no cells loss compared to untreated controls (FIGS. 22A and 22C, respectively), indicating no toxicity of the compounds at the doses tested. However, (−)-85a produced some incremental toxicity starting at 20 μM dose (FIG. 22B). The potential neuroprotective effects of (−)-81b, (−)-85a and (−)-85c against 6-OHDA-induced toxicity were evaluated next. Thus, pre-treatment of the cells with the test compounds for 24 h followed by exposure to 6-OHDA 75 μM for another 24 h produced dose-dependent protection of the cells from the neurotoxic insult and the greatest protective effect was observed at 5 μM for (−)-81b and 20 μM for (−)-85c. At those concentration both drugs increased cell survival by ˜20% compared to 6-OHDA (75 μM) treated alone (FIGS. 22D and 22F), indicating neuroprotective effect of these drugs. Compound (−)-85a also revealed dose-dependent neuroprotection and the highest effect was observed at the dose of 10 μM (15%) but the effect was not observed at the higher doses (FIG. 22E). This could be due to the fact that the compound by itself produced toxicity to the cells at doses ≥20 μM and the presence of 6-OHDA enhanced the toxicity (FIG. 22B).

Effect of (−)-81b (D636) and (−)-85a (D653) on the Aggregation of α-Syn with 0.5% Seeding.

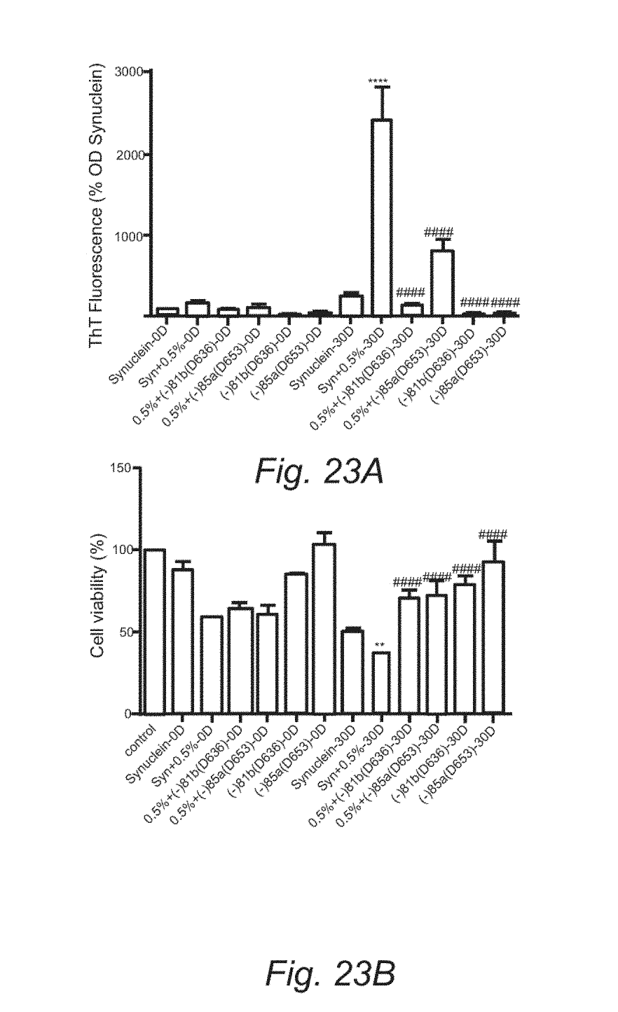

The α-syn assay was carried out by following the procedures developed by us earlier. The compounds (−)-81b (D636) and (−)-85a (D653) were incubated with α-syn seeded with 0.5% PFFs for a period of 30D at 37° C. without agitation. Seeding of monomeric α-syn with 0.5% PFFs leads to a significant increase in aggregation characterized by a 15-fold increase in ThT fluorescence at 30D. Both (−)-81b and (−)-85a lead to a significant decrease in the ThT fluorescence (p<0.0001) when compared to α-syn seeded with 0.5% PFFs alone at 30D (FIG. 23A). (−)-81b and (−)-85a showed a 16 fold and 3 fold decrease in ThT fluorescence respectively, when compared to seeded α-syn at 30D.

When PC12 cells were treated with the aggregates formed by 0.5% PFFs seeding at 30D, it was observed that the viability went down to 37% when compared to control. However, the seeded samples formed in the presence of (−)-81b and (−)-85a showed significant less toxicity when compared to seeded α-syn without compounds. The viability values of PC12 cells when treated with α-syn seeded samples in the presence of (−)-81b and (−)-85a were 71% and 72% at 30D when compared to control (FIG. 23B). We have previously shown that the parent compounds to our hybrid molecules, Pramipexole and 5-OHDPAT were far less effective in decreasing ThT fluorescence and reducing toxicities when compared to our multifunctional hybrid agonist.

In conclusion, the present study represents the development of a novel series of carbazole-based multifunctional DA D₂/D₃ receptor agonists. Compounds (−)-81b, (−)-85a and (−)-85c exhibited high binding affinity and full agonist activity at the both D₂ and D₃ receptors. The lead molecules were highly efficacious in a PD animal model by reversing hypo-locomotion activity with a long duration of action, indicating their potential in relieving motor dysfunction in PD. In a cellular model, the lead compounds exhibited potent anti-oxidant activity produced by neurotoxin 6-OHDA. To gain further insight into their possible multifunctional property, the data presented here also demonstrates that all three compounds (−)-81b, (−)-85a and (−)-85c exhibited neuroprotective property in an in vitro cellular model of PD by protecting neuronal PC12 cells from neurotoxin 6-OHDA. Furthermore, in vitro assays with recombinant α-syn protein demonstrated potent modulatory effect of (−)-91b and (−)-85a on aggregation and toxicity of α-syn protein. In summary, we are able to demonstrate that multifunctional drugs like (−)-91b, (−)-95a and (−)-95c have the potential not only to alleviate motor dysfunction in PD patients, but also to modify disease progression. More in depth future studies to establish the disease-modifying effects of the compounds will shed more light into their potential therapeutic application.

### Experimental Section

Reagents and solvents were obtained from commercial suppliers and used as received unless otherwise indicated. Dry solvent was obtained according to the standard procedure. All reactions were performed under N₂ atmosphere unless otherwise indicated. Analytical silica gel 60 F254-coated TLC plates were purchased from EMD Chemicals, Inc. and were visualized with UV light or by treatment with phosphomolybdic acid (PMA), or ninhydrin. Flash column chromatographic purification was done using Whatman Purasil 60A silica gel 230-400 mesh. Proton nuclear magnetic resonance (¹H NMR) spectra and carbon nuclear magnetic resonance (¹³C NMR) spectra were measured on Varian 400 and 600 MHz NMR spectrometer with tetramethylsilane (TMS) as an internal standard. The NMR solvent used was either CDCl₃ or CD₃OD unless otherwise indicated. Autopol III automatic polarimeter (Rudolph Research Analytical) was used to record the optical rotations. MEL-TEMP II (Laboratory Devices Inc., U.S.) capillary melting point apparatus was used to record the melting points. Purity of the compounds was determined by elemental analysis and was within ±0.4% of the theoretical value (≥95% purity). Elemental analyses were performed by Atlantic Microlab, Inc., GA, USA.

### Procedure A. 4′-Bromo-2-nitro-1,1′-biphenyl (72a)

To a stirring solution of 1-bromo-2-nitrobenzene (2.0 g, 9.9 mmol) and (4-bromophenyl)boronic acid (2.19 g, 10.89 mmol) in THF (25 mL) were added Pd(PPh₃)₄ (0.572 g, 0.50 mmol) followed by 2M K₂CO₃ (5.53 g in 20 mL water) at room temperature. The reaction mixture was stirred at 90° C. for 12 h after which it was cooled and extracted with CH₂Cl₂. The combined organic layer was dried over Na₂SO₄, filtered, and concentrated in vacuo. The crude material was purified by column chromatography over silica gel using hexane:ethyl acetate (9:1) as solvent to give compound 72a (2.7 g, 98%). ¹H NMR (600 MHz, CDCl₃): δ 7.88 (d, J=8.4 Hz, 1H), 7.64-7.61 (m, 1H), 7.59-7.55 (m, 2H), 7.53-7.50 (m, 1H), 7.42-7.40 (m, 1H), 7.20-7.18 (m, 2H).

### 2-Bromo-2′-nitro-1,1′-biphenyl (72c)

To a stirring solution of 1-bromo-2-nitrobenzene (2.50 g, 12.37 mmol) and (2-bromophenyl)boronic acid (2.73 g, 13.61 mmol) in THF (30 mL) were added Pd(PPh₃)₄ (0.715 g, 0.61 mmol) followed by 2M K₂CO₃ (5.53 g in 20 mL water) according to procedure A to give compound 72c (2.18 g, 94%). ¹H NMR (600 MHz, CDCl₃): δ 7.87 (d, J=8.4 Hz, 1H), 7.44 (t, J=7.2 Hz, 1H), 7.40 (d, J=9 Hz, 1H), 7.30 (d, J=7.8 Hz, 1H), 7.14 (t, J=7.2 Hz, 1H), 7.10 (d, J=7.2 Hz, 1H), 7.03 (d, J=7.2 Hz, 2H).

### Procedure B. 2-Bromo-9H-carbazole (73a)

Compound 72a and PPh₃ were dissolved in 1,2-dichlorobenzene and the resulting solution was stirred at 170° C. for 12 h after which it was cooled and extracted with CH₂Cl₂/H₂O. The combined organic layer was dried over Na₂SO₄, filtered, and concentrated in vacuo. The crude material was purified by column chromatography over silica gel using hexane:ethyl acetate (9:1) to yield compound 73a (1.57 g, 89%). ¹H NMR (600 MHz, CDCl₃): δ 8.04 (d, J=8.4 Hz, 1H), 7.92 (d, J=8.4 Hz, 1H), 7.62-7.58 (m, 2H), 7.52-7.47 (m, 2H), 7.39 (d, J=8.4 Hz, 1H).

### 4-Bromo-9H-carbazole (73c)

Compound 72c and PPh₃ were reacted in 1,2-dichlorobenzene according to procedure B to yield compound 73c (1.25 g, 87%). ¹H NMR (600 MHz, CDCl₃): δ 8.81 (d, J=7.8 Hz, 1H), 7.97 (s, 1H), 7.50 (t, J=7.2 Hz, 1H), 7.44 (d, J=7.2 Hz, 1H), 7.35 (t, J=7.8 Hz, 1H), 7.30 (d, J=7.8 Hz, 1H), 7.25-7.20 (m, 1H).

### Procedure C. tert-Butyl 2-bromo-9H-carbazole-9-carboxylate (74a)

To a stirring solution of compound 73a (1.5 g, 6.09 mmol) in THF (20 mL) were added (Boc)₂O (1.46 g, 6.7 mmol) and DMAP (0.819 g, 6.7 mmol) in THF (20 mL) at room temperature. The reaction mixture was stirred at the same temperature for 12 h. The crude mixture was evaporated under reduced pressure, followed by extraction with EtOAc (3×20 mL) in water. The combined organic layer was dried over Na₂SO₄, filtered, and concentrated in vacuo. The crude material was purified by silica gel column chromatography (hexane:EtOAc=19:1) to yield compound 74a (1.78 g, 84%). ¹H NMR (600 MHz, CDCl₃): δ 8.48 (d, J=8.4 Hz, 1H), 8.07 (d, J=8.4 Hz, 1H), 7.24 (t, J=7.8 Hz, 1H), 7.18 (d, J=7.8 Hz, 1H), 7.11 (t, J=7.2 Hz, 1H), 6.97 (t, J=8.10 Hz, 1H), 1.53 (s, 9H).

### tert-Butyl 3-bromo-9H-carbazole-9-carboxylate (74b)

Commercially available 3-Bromo-9H-carbazole (73b) (2.0 g, 8.13 mmol) was reacted with (Boc)₂O (1.95 g, 8.94 mmol) and DMAP (1.09 g, 8.94 mmol) in THF (20 mL) according to procedure C. The crude material was purified by column chromatography over silica gel using hexane:ethyl acetate (19:1) as solvent to give compound 74b (2.8 g, −100%). ¹H NMR (600 MHz, CDCl₃): δ 8.27 (d, J=8.4 Hz, 1H), 8.19 (d, J=8.4 Hz, 1H), 8.08 (d, J=2.4 Hz, 1H), 7.92 (qd, J=7.8, 0.6 Hz, 1H), 7.55 (dd, J=6.6, 2.4 Hz, 1H), 7.49 (td, J=8.4, 1.2 Hz, 1H), 7.36 (td, J=8.4, 1.2 Hz, 1H), 1.76 (s, 9H).

### tert-Butyl 4-bromo-9H-carbazole-9-carboxylate (74c)

Compound 73c (1.5 g, 6.09 mmol) was reacted with (Boc)₂O (1.46 g, 6.7 mmol) and DMAP (0.819 g, 6.7 mmol) in THF (20 mL) according to procedure C. The crude material was purified by silica gel column chromatography (hexane:EtOAc=19:1) to yield compound 74c (1.78 g, 84%). ¹H NMR (600 MHz, CDCl₃): δ 8.53 (s, 1H), 8.26 (d, J=8.4 Hz, 1H), 7.94 (d, J=7.8 Hz, 1H), 7.82 (d, J=8.4 Hz, 1H), 7.49-7.46 (m, 2H), 7.36 (t, J=7.2 Hz, 1H), 1.77 (s, 9H).

### Procedure D. tert-Butyl 2-(4-(2-((tert-butyldimethylsilyl)oxy)ethyl)piperazin-1-yl)-9H-carbazole-9-carboxylate (75a)

To a mixture of compounds 74a (0.8 g, 2.31 mmol), 1-(2-((tert-butyldimethylsilyl)oxy)-ethyl)piperazine (1.13 g, 4.62 mmol), BINAP (0.144 g, 0.23 mmol) and Cs₂CO₃ (2.26 g, 6.93 mmol), toluene (15 mL) was added under N₂ atmosphere. The reaction mixture was degassed by bubbling N₂ for 5 min and then Pd(OAc)₂ (0.039 g, 0.17 mmol) was added quickly. The system was degassed again and refluxed for 24 h under inert condition. The reaction mixture was cooled to room temperature, filtered through a pad of celite, washed with dichloromethane and concentrated in vacuum. The crude residue was purified by column chromatography (hexane:EtOAc=4:1) to afford compound 75a (0.97 g, 82%). ¹H NMR (600 MHz, CDCl₃): δ 8.23 (d, J=7.8 Hz, 1H), 7.92 (s, 1H), 7.84 (d, J=7.2 Hz, 1H), 7.81 (d, J=9.0 Hz, 1H), 7.35 (t, J=7.2 Hz, 1H), 7.29 (t, J=7.2 Hz, 1H), 7.00 (dd, J=9.0, 1.8 Hz, 1H), 3.82 (t, J=6.6 Hz, 2H), 3.32 (t, J=4.8 Hz, 4H), 2.74 (t, J=4.8 Hz, 4H), 2.62 (t, J=6.6 Hz, 2H), 1.75 (s, 9H), 0.91 (s, 9H), 0.09 (s, 6H).

### tert-Butyl 3-(4-(2-((tert-butyldimethylsilyl)oxy)ethyl)piperazin-1-yl)-9H-carbazole-9-carboxylate (75b)

A mixture of compound 74b (1.2 g, 3.47 mmol), 1-(2-((tert-butyldimethylsilyl)oxy)ethyl)piperazine (1.27 g, 5.20 mmol), Pd(OAc)₂ (0.058 g, 0.26 mmol), BINAP (0.216 g, 0.35 mmol) and Cs₂CO₃ (3.39 g, 10.4 mmol) in toluene (25 mL) was heated at 110° C. for 24 h according to procedure D. The crude material was purified by silica gel column chromatography (hexane:EtOAc=4:1) to give compound 75b (1.4 g, 79%). ¹H NMR (600 MHz, CDCl₃): δ 8.27 (d, J=8.4 Hz, 1H), 8.16 (d, J=8.4 Hz, 1H), 7.93 (d, J=7.8 Hz, 1H), 7.48 (d, J=2.4 Hz, 1H), 7.44 (t, J=7.8 Hz, 1H), 7.32 (t, J=7.8 Hz, 1H), 7.13 (dd, J=6.6, 2.4 Hz, 1H), 3.83 (t, J=6.6 Hz, 2H), 3.28 (t, J=4.8 Hz, 4H), 2.77 (t, J=4.8 Hz, 4H), 2.63 (t, J=6.6 Hz, 2H), 1.75 (s, 9H), 0.92 (s, 9H), 0.09 (s, 6H).

### tert-Butyl 4-(4-(2-((tert-butyldimethylsilyl)oxy)ethyl)piperazin-1-yl)-9H-carbazole-9-carboxylate (75c)

A mixture of 74c (2.70 g, 7.80 mmol), 1-(2-((tert-butyldimethylsilyl)oxy)-ethyl)piperazine (3.24 g, 13.25 mmol), Pd(OAc)₂ (0.0.13 g, 0.59 mmol), BINAP (0.49 g, 0.78 mmol) and Cs₂CO₃ (7.62 g, 23.4 mmol) in toluene (30 mL) was heated at 110° C. for 24 h according to procedure D. The crude residue was purified by column chromatography (hexane:EtOAc=4:1) to afford compound 75c (3.24 g, 82%). ¹H NMR (600 MHz, CDCl₃): δ 8.34 (dd, J=8.1, 4.8 Hz, 2H), 8.08 (d, J=8.4 Hz, 1H), 7.45 (t, J=7.2 Hz, 1H), 7.41-7.36 (m, 2H), 7.04 (d, J=8.4 Hz, 1H), 3.85 (t, J=6.6 Hz, 2H), 3.33 (d, J=5.4 Hz, 2H), 2.99 (t, J=5.4 Hz, 2H), 2.71 (t, J=6.6 Hz, 4H), 1.75 (s, 9H), 0.91 (s, 9H), 0.10 (s, 6H).

### Procedure E. tert-Butyl 2-(4-(2-hydroxyethyl)piperazin-1-yl)-9H-carbazole-9-carboxylate (76a)

Into a stirring solution of compound 75a (0.95 g, 1.86 mmol) in THF (10 mL) was added n-tetrabutylammonium fluoride (2.8 mL, 2.8 mmol, 1.0 M solution in THF) at 0° C. The reaction mixture was then stirred at room temperature for 2 h. THF was evaporated in vacuo, and the residue was diluted with CH₂Cl₂ (25 mL) and washed with a saturated solution of NaHCO₃. The water layer was extracted with CH₂Cl₂ (3×50 mL). The combined organic layer was washed with brine, dried over Na₂SO₄, and evaporated under reduced pressure. The crude product was purified by silica gel column chromatography (EtOAc:MeOH=9:1) to give compound 76a (0.595 g, 81%). ¹H NMR (600 MHz, CDCl₃): δ 8.23 (d, J=7.8 Hz, 1H), 7.94 (s, 1H), 7.85 (d, J=7.8 Hz, 1H), 7.82 (d, J=9.0 Hz, 1H), 7.36 (t, J=7.2 Hz, 1H), 7.30 (t, J=7.2 Hz, 1H), 7.00 (dd, J=6.6, 1.8 Hz, 1H), 3.68 (t, J=5.4 Hz, 2H), 3.34 (t, J=4.8 Hz, 4H), 2.73 (t, J=4.8 Hz, 4H), 2.64 (t, J=5.4 Hz, 2H), 1.76 (s, 9H).

### tert-Butyl 3-(4-(2-hydroxyethyl)piperazin-1-yl)-9H-carbazole-9-carboxylate (76b)

Compound 75b (1.2 g, 2.35 mmol) in THF (15 mL) was reacted with n-tetrabutylammonium fluoride (4.71 mL, 4.71 mmol, 1.0 M solution in THF) according to procedure E. The crude product was purified by silica gel column chromatography (EtOAc:MeOH=9:1) to yield compound 76b (0.78 g, 84%). ¹H NMR (600 MHz, CDCl₃): δ 8.28 (d, J=8.4 Hz, 1H), 8.18 (d, J=8.4 Hz, 1H), 7.93 (d, J=7.8 Hz, 1H), 7.49 (d, J=2.4 Hz, 1H), 7.44 (t, J=7.8 Hz, 1H), 7.33 (t, J=7.8 Hz, 1H), 7.13 (dd, J=6.6, 2.4 Hz, 1H), 3.69 (t, J=5.4 Hz, 2H), 3.29 (t, J=4.8 Hz, 4H), 2.76 (t, J=4.8 Hz, 4H), 2.66 (t, J=5.4 Hz, 2H), 1.75 (s, 9H).

### tert-Butyl 4-(4-(2-hydroxyethyl)piperazin-1-yl)-9H-carbazole-9-carboxylate (76c)

Compound 75c (3.30 g, 6.47 mmol) was reacted with n-tetrabutylammonium fluoride (9.70 mL, 9.70 mmol, 1.0 M solution in THF) in THF (30 mL) according to procedure E. The crude product was purified by silica gel column chromatography (EtOAc:MeOH=9:1) to give compound 76c (2.01 g, 80%). ¹H NMR (600 MHz, CDCl₃): δ 8.27 (d, J=6.6 Hz, 1H), 8.17 (d, J=7.8 Hz, 1H), 7.92 (d, J=7.8 Hz, 1H), 7.47 (s, 1H), 7.43 (t, J=7.2 Hz, 1H), 7.31 (t, J=7.2 Hz, 1H), 7.11 (d, J=9.0 Hz, 1H), 3.69 (t, J=5.4 Hz, 2H), 3.26 (s, 4H), 2.76 (s, 4H), 2.62 (t, J=5.4 Hz, 2H), 1.73 (s, 9H).

### Procedure F. tert-Butyl 2-(4-(2-oxoethyl)piperazin-1-yl)-9H-carbazole-9-carboxylate (77a)

Into a stirring solution of compound 76a (0.30 g, 0.76 mmol) in CH₂Cl₂ (6 mL) and DMSO (3 mL), was added Et₃N (0.74 mL, 5.31 mmol) at 0° C. The reaction mixture was stirred for 5 min followed by addition of SO₃.py complex (0.604 g, 3.79 mmol) at 0° C. Ice bath was removed and the reaction mixture was stirred at room temperature for 2 h. The reaction mixture was quenched by addition of water and extracted with CH₂Cl₂ (3×30 mL). The combined organic layer was dried using Na₂SO₄, and the solvent was removed under reduced pressure. The crude product was purified by silica gel column chromatography (hexane:EtOAc=3:7) to give aldehyde 77a (0.25 g, 84%). The purified aldehyde was used immediately for next step. ¹H NMR (600 MHz, CDCl₃): δ 9.74 (s, 1H), 8.23 (d, J=8.4 Hz, 1H), 7.94 (s, 1H), 7.84 (d, J=7.8 Hz, 1H), 7.80 (d, J=8.4 Hz, 1H), 7.36 (td, J=7.2, 1.2 Hz, 1H), 7.29 (td, J=7.2, 1.2 Hz, 1H), 6.97 (dd, J=6.6, 1.8 Hz, 1H), 3.36 (t, J=4.8 Hz, 4H), 3.24 (t, J=1.2 Hz, 2H), 2.73 (t, J=4.8 Hz, 4H), 1.75 (s, 9H).

### tert-Butyl 3-(4-(2-oxoethyl)piperazin-1-yl)-9H-carbazole-9-carboxylate (77b)

Compound 76b (0.45 g, 1.14 mmol) in CH₂Cl₂ (10 mL) and DMSO (5 mL), was oxidized using SO₃.py complex (0.905 g, 5.69 mmol) and Et₃N (1.11 mL, 7.96 mmol) according to procedure F. The crude product was purified by silica gel column chromatography (EtOAc) to yield compound 77b (0.35 g, 78%). ¹H NMR (600 MHz, CDCl₃): δ 9.76 (s, 1H), 8.28 (d, J=8.4 Hz, 1H), 8.18 (d, J=8.4 Hz, 1H), 7.93 (d, J=7.8 Hz, 1H), 7.48 (d, J=2.4 Hz, 1H), 7.44 (t, J=7.2 Hz, 1H), 7.32 (t, J=7.2 Hz, 1H), 7.12 (dd, J=6.6, 2.4 Hz, 1H), 3.31 (t, J=4.2 Hz, 4H), 3.25 (t, J=1.2 Hz, 2H), 2.74 (t, J=4.2 Hz, 4H), 1.74 (s, 9H).

### tert-Butyl 4-(4-(2-oxoethyl)piperazin-1-yl)-9H-carbazole-9-carboxylate (77c)

Alcohol 76c (0.35 g, 0.88 mmol) was oxidized using SO₃.py complex (0.704 g, 4.425 mmol), DMSO (9 mL) and Et₃N (0.86 mL, 6.19 mmol) in CH₂Cl₂ (6 mL) according to procedure F. The crude product was purified by silica gel column chromatography (hexane:EtOAc=3:7) to give aldehyde 77c (0.31 g, 89%). The purified aldehyde was used immediately for next step. ¹H NMR (600 MHz, CDCl₃): δ 9.78 (s, 1H), 8.33 (d, J=7.8 Hz, 1H), 8.27 (d, J=7.8 Hz, 1H), 7.84 (d, J=7.8 Hz, 1H), 8.09 (d, J=8.4 Hz, 1H), 7.44-7.40 (m, 2H), 7.35 (t, J=7.2 Hz, 1H), 7.06 (dd, J=7.8, 1.8 Hz, 1H), 3.36-3.34 (m, 4H), 3.06 (t, J=11.1 Hz, 2H), 2.73 (d, J=11.4 Hz, 2H), 2.71 (d, J=10.2 Hz, 2H), 1.75 (s, 9H).

### Procedure G. tert-Butyl 2-(4-(2-((2-amino-4,5,6,7-tetrahydrobenzo[d]thiazol-6-yl)(propyl)amino)-ethyl)piperazin-1-yl)-9H-carbazole-9-carboxylate (78a)

Into a stirring solution of racemic N⁶-propyl-4,5,6,7-tetrahydrobenzo[d]thiazole-2,6-diamine (0.058 g, 0.27 mmol) in CH₂Cl₂ (7 mL) was added aldehyde 77a (0.12 g, 0.31 mmol). After the mixture was stirred for 1.5 h, NaBH(OAc)₃ (0.13 g, 0.61 mmol) was added and the mixture was stirred for another 46 h at room temperature. The reaction mixture was quenched with a saturated solution of NaHCO₃ at 0° C. and extracted with CH₂Cl₂ (3×50 mL). The combined organic layer was dried over Na₂SO₄, and the solvent was removed under reduced pressure. The crude product was purified by silica gel column chromatography (CH₂Cl₂:MeOH=19:1) to afford compound 78a (0.06 g, 38%). ¹H NMR (600 MHz, CDCl₃): δ 8.23 (d, J=8.4 Hz, 1H), 7.92 (s, 1H), 7.85 (d, J=7.8 Hz, 1H), 7.81 (d, J=8.4 Hz, 1H), 7.35 (td, J=7.2, 1.2 Hz, 1H), 7.29 (td, J=7.2, 1.2 Hz, 1H), 7.00 (dd, J=6.0, 2.4 Hz, 1H), 4.77 (bs, 2H), 3.33 (t, J=4.8 Hz, 4H), 3.07 (m, 1H), 2.78-2.68 (m, 8H), 2.60-2.47 (m, 6H), 2.02-2.00 (m, 1H), 1.75 (s, 10H), 1.51-1.47 (m, 2H), 0.90 (t, J=7.2 Hz, 3H).

### tert-Butyl 3-(4-(2-((2-amino-4,5,6,7-tetrahydrobenzo[d]thiazol-6-yl)(propyl)amino)ethyl)piperazin-1-yl)-9H-carbazole-9-carboxylate (78b)

Aldehyde 77b (0.15 g, 0.38 mmol) in CH₂Cl₂ (10 mL) was reacted with racemic N⁶-propyl-4,5,6,7-tetrahydrobenzo[d]thiazole-2,6-diamine (0.073 g, 0.34 mmol) and NaBH(OAc)₃ (0.162 g, 0.76 mmol) according to procedure G. Crude product was purified by column chromatography (EtOAc:MeOH=9:1) to afford compound 78b (0.065 g, 32%). ¹H NMR (400 MHz, CDCl₃): δ 8.27 (d, J=8.0 Hz, 1H), 8.16 (d, J=9.2 Hz, 1H), 7.93 (d, J=7.6 Hz, 1H), 7.48 (d, J=2.4 Hz, 1H), 7.44 (t, J=7.2 Hz, 1H), 7.32 (t, J=7.2 Hz, 1H), 7.12 (dd, J=7.2, 2.4 Hz, 1H), 5.12 (bs, 2H), 3.28 (t, J=4.8 Hz, 4H), 3.07-3.02 (m, 1H), 2.78-2.67 (m, 8H), 2.58-2.45 (m, 6H), 2.00-1.98 (m, 1H), 1.74 (s, 10H), 1.52-1.46 (m, 2H), 0.90 (t, J=7.2 Hz, 3H).

### tert-Butyl 4-(4-(2-((2-amino-4,5,6,7-tetrahydrobenzo[d]thiazol-6-yl)(propyl)amino)ethyl)piperazin-1-yl)-9H-carbazole-9-carboxylate (78c)

Aldehyde 77c (0.15 g, 0.38 mmol) was reacted with racemic N⁶-propyl-4,5,6,7-tetrahydrobenzo[d]thiazole-2,6-diamine (0.073 g, 0.34 mmol) and NaBH(OAc)₃ (0.162 g, 0.76 mmol) in CH₂Cl₂ (10 mL) according to procedure G. The crude product was purified by silica gel column chromatography (CH₂Cl₂:MeOH=19:1) to afford compound 78c (0.91 g, 41%). ¹H NMR (600 MHz, CDCl₃): δ 8.23 (d, J=6.6 Hz, 1H), 8.28 (d, J=6.6 Hz, 1H), 8.06 (d, J=6.6 Hz, 1H), 7.43 (d, J=5.4 Hz, 1H), 7.37 (d, J=5.4 Hz, 2H), 7.02 (d, J=6.0 Hz, 1H), 5.03 (s, 2H), 3.10-3.05 (m, 3H), 3.07-2.98 (m, 2H), 2.76-2.69 (m, 4H), 2.62-2.60 (m, 8H), 2.01-2.00 (m, 1H), 1.73 (s, 10H), 1.50-1.46 (m, 2H), 0.90 (t, J=7.2 Hz, 3H).

### (S)-tert-Butyl 2-(4-(2-((2-amino-4,5,6,7-tetrahydrobenzo[d]thiazol-6-yl)(propyl)amino)ethyl)piperazin-1-yl)-9H-carbazole-9-carboxylate (79a)

Aldehyde 77a (0.125 g, 0.32 mmol) was reacted with (−)-pramipexole (0.06 g, 0.29 mmol) and NaBH(OAc)₃ (0.135 g, 0.64 mmol) in CH₂Cl₂ (8 mL) according to procedure G. The crude product was purified by silica gel column chromatography (CH₂Cl₂:MeOH=19:1) to afford compound 79a (0.067 g, 40%). ¹H NMR (600 MHz, CDCl₃): δ 8.23 (d, J=8.4 Hz, 1H), 7.92 (s, 1H), 7.85 (d, J=7.2 Hz, 1H), 7.81 (d, J=8.4 Hz, 1H), 7.35 (td, J=7.2, 1.2 Hz, 1H), 7.29 (td, J=7.2, 1.2 Hz, 1H), 7.00 (dd, J=6.6, 2.4 Hz, 1H), 4.76 (bs, 2H), 3.33 (t, J=4.8 Hz, 4H), 3.07 (m, 1H), 2.79-2.68 (m, 8H), 2.61-2.48 (m, 6H), 2.02-2.00 (m, 1H), 1.75 (s, 10H), 1.51-1.48 (m, 2H), 0.90 (t, J=7.2 Hz, 3H); [α]_{D}²⁵=−44.0 (c=1.0 in CH₂Cl₂).

### (S)-tert-Butyl 3-(4-(2-((2-amino-4,5,6,7-tetrahydrobenzo[d]thiazol-6-yl)(propyl)amino)ethyl)piperazin-1-yl)-9H-carbazole-9-carboxylate (79b)

Aldehyde 77b (0.30 g, 0.76 mmol) was reacted with (−)-pramipexole (0.145 g, 0.69 mmol) and NaBH(OAc)₃ (0.323 g, 1.52 mmol) in CH₂Cl₂ (15 mL) according to procedure G. The crude product was purified by silica gel column chromatography (EtOAc:MeOH=9:1) to afford compound 79b (0.158 g, 39%). ¹H NMR (600 MHz, CDCl₃): δ 8.27 (d, J=7.2 Hz, 1H), 8.16 (d, J=8.4 Hz, 1H), 7.93 (d, J=7.8 Hz, 1H), 7.48 (d, J=2.4 Hz, 1H), 7.43 (t, J=7.2 Hz, 1H), 7.32 (t, J=7.2 Hz, 1H), 7.12 (dd, J=6.6, 2.4 Hz, 1H), 5.21 (bs, 2H), 3.28 (t, J=4.8 Hz, 4H), 3.07-3.02 (m, 1H), 2.78-2.66 (m, 8H), 2.59-2.46 (m, 6H), 2.01-1.99 (m, 1H), 1.74 (s, 10H), 1.52-1.46 (m, 2H), 0.90 (t, J=7.2 Hz, 3H).

### (S)-tert-Butyl 4-(4-(2-((2-amino-4,5,6,7-tetrahydrobenzo[d]thiazol-6-yl)(propyl)amino)ethyl)piperazin-1-yl)-9H-carbazole-9-carboxylate (79c)

Aldehyde 77c (0.150 g, 0.38 mmol) was reacted with (−)-pramipexole (0.73 g, 0.54 mmol) and NaBH(OAc)₃ (0.162 g, 0.76 mmol) in CH₂Cl₂ (10 mL) according to procedure G. The crude product was purified by silica gel column chromatography (CH₂Cl₂:MeOH=19:1) to afford compound 79c (0.097 g, 43%). ¹H NMR (600 MHz, CDCl₃): δ 8.32 (d, J=7.2 Hz, 1H), 8.28 (d, J=7.8 Hz, 1H), 8.06 (d, J=7.8 Hz, 1H), 7.43 (d, J=7.2 Hz, 1H), 7.40-7.35 (m, 2H), 7.30 (d, J=7.2 Hz, 1H), 4.83 (bs, 2H), 3.33 (d, J=8.4 Hz, 2H), 3.06 (m, 3H), 2.99 (m, 2H), 2.76-2.70 (m, 4H), 2.60 (m, 7H), 1.74 (s, 10H), 1.51-1.48 (m, 2H), 0.90 (t, J=6.6 Hz, 3H); [α]_{D}²⁵=−27.20 (c=1.0 in CH₂Cl₂).

### Procedure H. N⁶-(2-(4-(9H-carbazol-2-yl)piperazin-1-yl)ethyl)-N⁶-propyl-4,5,6,7-tetrahydrobenzo-[d]thiazole-2,6-diamine (80a) (D-652)

To a stirred solution of 78a (0.055 g, 0.09 mmol) in CH₂Cl₂ (3 mL) at 0° C., trifluoroacetic acid (3 mL) was added slowly and the reaction mixture was stirred for 3 h at room temperature. Unreacted TFA and solvent were removed under reduced pressure and the obtained TFA salt was washed with ether for several times followed by drying to yield 80a (0.079 g, 90%). ¹H NMR (600 MHz, CD₃OD): δ 7.99 (d, J=8.4 Hz, 1H), 7.96 (d, J=7.8 Hz, 1H), 7.42 (d, J=7.8 Hz, 1H), 7.32 (t, J=7.2 Hz, 1H), 7.25 (s, 1H), 7.13 (t, J=7.2 Hz, 1H), 7.03 (d, J=8.4 Hz, 1H), 3.84 (m, 1H), 3.64-3.55 (m, 5H), 3.48-3.42 (m, 3H), 3.36 (s, 4H), 3.23-3.14 (m, 2H), 3.01-2.99 (m, 1H), 2.84-2.79 (m, 1H), 2.71-2.65 (m, 2H), 2.32-2.31 (m, 1H), 2.02-1.95 (m, 1H), 1.83-1.79 (m, 2H), 1.03 (t, J=7.2 Hz, 3H); ¹³C NMR (150 MHz, CD₃OD): δ 170.28, 160.68, 145.32, 140.57, 132.69, 125.56, 124.53, 122.50, 121.20, 120.14, 119.84, 119.33, 118.79, 118.29, 117.08, 115.15, 111.37, 110.77, 109.73, 59.75, 52.49, 51.56, 50.08, 22.29, 21.88, 21.39, 18.01, 10.19; Anal. Calcd for C₂₈H₃₆N₆S.4CF₃COOH: C, 45.77; H, 4.27; N, 8.90. Found: C, 45.71; H, 4.64; N, 8.87.

### N⁶-(2-(4-(9H-carbazol-3-yl)piperazin-1-yl)ethyl)-N⁶-propyl-4,5,6,7-tetrahydrobenzo[d]thiazole-2,6-diamine (80b) (D-627)

Compound 78b (0.05 g, 0.09 mmol) in CH₂Cl₂ (2 mL) at 0° C., was treated with trifluoroacetic acid (2 mL) according to procedure H to obtain the TFA salt of compound 80b (0.052 g, 84%). ¹H NMR (400 MHz, CD₃OD): δ 8.01 (d, J=8.0 Hz, 1H), 7.66 (d, J=1.6 Hz, 1H), 7.41-7.36 (m, 2H), 7.32 (t, J=7.2 Hz, 1H), 7.16-7.14 (m, 1H), 7.11 (t, J=7.2 Hz, 1H), 3.34 (s, 1H), 3.19 (s, 4H), 2.98-2.91 (m, 2H), 2.80-2.51 (m, 12H), 2.07-2.04 (m, 1H), 1.81-1.77 (m, 1H), 1.63-1.53 (m, 2H), 0.95 (t, J=7.2 Hz, 3H); ¹³C NMR (100 MHz, CD₃OD): δ 168.63, 144.25, 143.69, 143.37, 140.69, 135.84, 125.07, 123.21, 122.92, 119.50, 118.21, 118.00, 113.45, 110.87, 110.41, 107.90, 58.85, 55.41, 53.24, 53.12, 51.07, 25.37, 24.70, 23.79, 20.35, 10.43; Anal. Calcd for C₂₈H₃₆N₆S.2CF₃COOH: C, 53.62; H, 5.34; N, 11.73. Found: C, 53.64; H, 5.83; N, 11.34.

### N⁶-(2-(4-(9H-carbazol-4-yl)piperazin-1-yl)ethyl)-N⁶-propyl-4,5,6,7-tetrahydrobenzo[d]thiazole-2,6-diamine (80c) (D-658)

Compound 78c (0.082 g, 0.14 mmol) was treated with trifluoroacetic acid (3 mL) in CH₂Cl₂ (3 mL) according to procedure H to furnish the TFA salt of 80c (0.091 g, 90%). ¹H NMR (600 MHz, CD₃OD): δ 8.08 (d, J=7.8 Hz, 1H), 7.45 (d, J=7.8 Hz, 1H), 7.36 (t, J=7.8 Hz, 1H), 7.31 (t, J=7.8 Hz, 1H), 7.22 (d, J=7.8 Hz, 1H), 7.19 (t, J=7.2 Hz, 1H), 6.82 (d, J=7.8 Hz, 1H), 3.94-3.89 (m, 1H), 3.83 (d, J=7.2 Hz, 2H), 3.77-3.59 (m, 8H), 3.29 (d, J=1.2 Hz, 1H), 3.24-3.20 (m, 3H), 3.06-3.04 (m, 1H), 2.92-2.88 (m, 1H), 2.78-2.71 (m, 2H), 2.37-2.36 (m, 1H), 2.11-2.04 (m, 1H), 1.86-1.82 (m, 2H), 1.04 (t, J=7.2 Hz, 3H); ¹³C NMR (150 MHz, CD₃OD): δ 170.32, 161.19, 146.42, 141.50, 139.78, 132.81, 125.91, 124.74, 121.78, 121.34, 118.62, 115.64, 111.66, 110.23, 107.39, 59.17, 53.36, 53.25, 52.80, 50.81, 48.60, 48.01, 44.95, 22.51, 22.03, 21.49, 18.34, 9.79; Anal. Calcd for C₂₈H₃₆N₆S, 3CF₃COOH, CH₃OH, H₂O: C, 47.73; H, 5.15; N, 9.54. Found: C, 47.63; H, 4.67; N, 9.23.

### (S)—N⁶-(2-(4-(9H-carbazol-2-yl)piperazin-1-yl)ethyl)-N⁶-propyl-4,5,6,7-tetrahydrobenzo[d]thiazole-2,6-diamine (81a) (D-651)

Compound 79a (0.065 g, 0.11 mmol) was treated with trifluoroacetic acid (3 mL) in CH₂Cl₂ (3 mL) according to procedure H to furnish the TFA salt of 81a (0.085 g, 93%). ¹H NMR (600 MHz, CD₃OD): δ 7.99 (d, J=8.4 Hz, 1H), 7.96 (d, J=7.8 Hz, 1H), 7.42 (d, J=7.8 Hz, 1H), 7.33 (t, J=7.2 Hz, 1H), 7.26 (s, 1H), 7.13 (t, J=7.2 Hz, 1H), 7.04 (d, J=8.4 Hz, 1H), 3.86 (m, 1H), 3.65-3.56 (m, 5H), 3.49-3.42 (m, 3H), 3.37 (s, 4H), 3.25-3.16 (m, 2H), 3.02-3.00 (m, 1H), 2.85-2.81 (m, 1H), 2.73-2.66 (m, 2H), 2.34-2.32 (m, 1H), 2.04-1.97 (m, 1H), 1.83-1.80 (m, 2H), 1.03 (t, J=7.2 Hz, 3H); ¹³C NMR (150 MHz, CD₃OD): δ 170.29, 160.41, 145.25, 140.58, 132.67, 125.55, 124.55, 122.49, 121.21, 120.15, 119.82, 119.32, 118.82, 118.24, 116.97, 115.06, 111.39, 110.76, 109.70, 59.75, 52.50, 51.55, 50.17, 22.31, 21.89, 21.40, 17.98, 10.18; [α]_{D}²⁵=−24.8 (c=1.0 in CH₃OH); Anal. Calcd for C₂₈H₃₆N₆S.3CF₃COOH.2H₂O: C, 47.11; H, 5.00; N, 9.70. Found: C, 47.63; H, 4.67; N, 9.23.

### (S)—N⁶-(2-(4-(9H-carbazol-3-yl)piperazin-1-yl)ethyl)-N⁶-propyl-4,5,6,7-tetrahydrobenzo[d]thiazole-2,6-diamine (81b) (D-636)

Compound 79b (0.15 g, 0.25 mmol) was treated with trifluoroacetic acid (8 mL) in CH₂Cl₂ (8 mL) according to procedure H to furnish the TFA salt of 81b (0.20 g, 96%). ¹H NMR (600 MHz, CD₃OD): δ 8.27 (s, 1H), 8.11 (d, J=7.2 Hz, 1H), 7.59-7.54 (m, 2H), 7.49 (d, J=8.4 Hz, 1H), 7.44 (t, J=7.2 Hz, 1H), 7.21 (t, J=7.2 Hz, 1H), 3.96 (m, 1H), 3.80 (s, 4H), 3.57-3.45 (m, 2H), 3.34-3.27 (m, 2H), 3.20-3.10 (m, 7H), 2.97-2.92 (m, 1H), 2.83-2.75 (m, 2H), 2.44-2.42 (m, 1H), 2.17-2.10 (m, 1H), 1.90-1.84 (m, 2H), 1.07 (t, J=7.2 Hz, 3H); ¹³C NMR (150 MHz, CD₃OD): δ 170.35, 160.54, 141.04, 139.17, 135.08, 132.80, 126.39, 123.44, 122.29, 119.95, 119.05, 117.60, 111.67, 111.55, 111.45, 110.90, 59.31, 54.18, 53.61, 51.21, 50.61, 22.42, 22.09, 21.50, 17.98, 9.82; [α]_{D}²⁵=−17.4 (c=1.0 in CH₃OH); Anal. Calcd for C₂₈H₃₆N₆S.3CF₃COOH.H₂O: C, 48.11; H, 4.87; N, 9.90. Found: C, 48.25; H, 4.99; N, 9.58.

### (S)—N⁶-(2-(4-(9H-carbazol-4-yl)piperazin-1-yl)ethyl)-N⁶-propyl-4,5,6,7-tetrahydrobenzo[d]thiazole-2,6-diamine (81c) (D-657)

Compound 79c (0.83 g, 0.14 mmol) was treated with trifluoroacetic acid (7 mL) in CH₂Cl₂ (7 mL) according to procedure H to furnish the TFA salt of 81c (0.085 g, 93%). ¹H NMR (600 MHz, CD₃OD): δ 8.07 (d, J=7.2 Hz, 1H), 7.44 (d, J=7.8 Hz, 1H), 7.36 (d, J=7.8 Hz, 1H), 7.31 (t, J=7.8 Hz, 1H), 7.22 (d, J=7.8 Hz, 1H), 7.19 (t, J=7.2 Hz, 1H), 6.81 (d, J=8.4 Hz, 1H), 3.90 (s, 1H) 3.84 (d, J=6.6 Hz, 2H), 3.78-3.71 (m, 4H), 3.56-3.43 (m, 4H), 3.29 (s, 2H), 3.26-3.21 (m, 3H), 3.06-3.04 (m, 1H), 2.92-2.88 (m, 1H), 2.78-2.71 (m, 2H), 2.36 (bs, 1H), 2.09-2.04 (m, 1H), 1.86-1.82 (m, 2H), 1.04 (t, J=7.2 Hz, 3H); 13C NMR (150 MHz, CD₃OD): δ 170.32, 161.06, 146.42, 139.77, 132.80, 126.38, 124.29, 122.26, 121.30, 119.075, 118.18, 115.62, 111.61, 110.74, 109.72, 59.72, 58.72, 53.27, 52.76, 50.70, 44.91, 22.99, 22.34, 21.44, 18.30, 10.07, 9.46; [α]_{D}²⁵=−25.0 (c=1.0 in CH₃OH); Anal. Calcd for C₂₈H₃₆N₆S.3CF₃COOH: C, 49.16; H, 4.73; N, 10.12. Found: C, 48.93; H, 4.84; N, 9.77.

### tert-Butyl 2-(4-(2-((5-methoxy-1,2,3,4-tetrahydronaphthalen-2-yl)(propyl)amino)ethyl)-piperazin-1-yl)-9H-carbazole-9-carboxylate (82a)

Compound 77a (0.10 g, 0.25 mmol) was reacted with (+)-(5-methoxy-1,2,3,4-tetrahydro-naphthalen-2-yl)-propyl-amine (0.056 g, 0.25 mmol) and NaBH(OAc)₃ (0.108 g, 0.51 mmol) in CH₂Cl₂ (7 mL) according to procedure G. The crude product was purified by silica gel column chromatography (hexane:EtOAc=1:3) to afford compound 82a (0.115 g, 77%). ¹H NMR (600 MHz, CDCl₃): δ 8.23 (d, J=7.8 Hz, 1H), 7.91 (s, 1H), 7.83 (d, J=7.8 Hz, 1H), 7.79 (d, J=8.4 Hz, 1H), 7.34 (t, J=7.2, Hz, 1H), 7.28 (t, J=7.2 Hz, 1H), 7.08 (t, J=7.8 Hz, 1H), 6.98 (dd, J=6.6, 1.8 Hz, 1H), 6.72 (d, J=7.2, Hz, 1H), 6.64 (d, J=7.8, Hz, 1H), 3.79 (s, 3H), 3.32 (t, J=4.8 Hz, 4H), 3.02-2.93 (m, 2H), 2.88-2.85 (m, 1H), 2.79-2.75 (m, 3H), 2.70 (t, J=4.8 Hz, 4H), 2.56-2.50 (m, 5H), 2.09-2.06 (m, 1H), 1.74 (s, 9H), 1.62-1.55 (m, 1H), 1.53-1.47 (m, 2H), 0.91 (t, J=7.2 Hz, 3H).

### tert-Butyl 3-(4-(2-((5-methoxy-1,2,3,4-tetrahydronaphthalen-2-yl)(propyl)amino)ethyl)piperazin-1-yl)-9H-carbazole-9-carboxylate (82b)

Compound 77b (0.170 g, 0.43 mmol) was reacted with (+)-(5-methoxy-1,2,3,4-tetrahydro-naphthalen-2-yl)-propyl-amine (0.095 g, 0.43 mmol) and NaBH(OAc)₃ (0.183 g, 0.86 mmol) in CH₂Cl₂ (10 mL) according to procedure G. The crude product was purified by silica gel column chromatography with EtOAc to afford compound 82b (0.11 g, 43%). ¹H NMR (600 MHz, CDCl₃): δ 8.27 (d, J=7.8 Hz, 1H), 8.16 (d, J=8.4 Hz, 1H), 7.92 (d, J=7.8 Hz, 1H), 7.47 (d, J=2.4 Hz, 1H), 7.43 (t, J=7.2, Hz, 1H), 7.31 (t, J=7.2 Hz, 1H), 7.12 (dd, J=6.6, 2.4 Hz, 1H), 7.08 (t, J=8.4 Hz, 1H), 6.72 (d, J=7.2, Hz, 1H), 6.64 (d, J=8.4 Hz, 1H), 3.79 (s, 3H), 3.27 (t, J=4.8 Hz, 4H), 3.03-2.98 (m, 2H), 2.90-2.87 (m, 1H), 2.81-2.77 (m, 3H), 2.73 (t, J=4.8 Hz, 4H), 2.59-2.51 (m, 5H), 2.11-2.08 (m, 1H), 1.74 (s, 9H), 1.63-1.57 (m, 1H), 1.55-1.49 (m, 2H), 0.91 (t, J=7.2 Hz, 3H).

### tert-Butyl 4-(4-(2-((5-methoxy-1,2,3,4-tetrahydronaphthalen-2-yl)(propyl)amino)ethyl)piperazin-1-yl)-9H-carbazole-9-carboxylate (82c)

Compound 77c (0.10 g, 0.25 mmol) was reacted with (+)-(5-methoxy-1,2,3,4-tetrahydro-naphthalen-2-yl)-propyl-amine (0.055 g, 0.25 mmol) and NaBH(OAc)₃ (0.108 g, 0.50 mmol) in CH₂Cl₂ (8 mL) according to procedure G. The crude product was purified by silica gel column chromatography (hexane:EtOAc=1:3) to afford compound 82c (0.112 g, 74%). ¹H NMR (600 MHz, CDCl₃): δ 8.23 (d, J=8.4 Hz, 1H), 8.16 (d, J=8.4, Hz, 1H), 8.09 (d, J=7.2 Hz, 1H), 7.47 (d, J=7.8 Hz, 1H), 7.39 (m, 2H), 7.13 (t, J=7.8 Hz, 1H), 7.00 (d, J=7.8 Hz, 1H), 6.70 (q, J=7.8 Hz, 2H), 3.92 (bs, 2H), 3.79 (s, 3H), 3.58-3.48 (m, 4H), 2.29 (m, 2H), 3.06-3.04 (m, 5H), 2.62, (m, 1H), 2.37 (bs, 1H), 2.09-2.06 (m, 1H), 1.74 (s, 9H), 1.62-1.55 (m, 1H), 1.53-1.47 (m, 2H), 0.92 (t, J=7.2 Hz, 3H).

### (S)-tert-Butyl 2-(4-(2-((5-methoxy-1,2,3,4-tetrahydronaphthalen-2-yl)(propyl)amino)ethyl)piperazin-1-yl)-9H-carbazole-9-carboxylate (83a)

Compound 77a (0.10 g, 0.25 mmol) was reacted with (−)-(5-methoxy-1,2,3,4-tetrahydro-naphthalen-2-yl)-propyl-amine (0.056 g, 0.25 mmol) and NaBH(OAc)₃ (0.108 g, 0.51 mmol) in CH₂Cl₂ (7 mL) according to procedure G. The crude product was purified by silica gel column chromatography (hexane:EtOAc=1:3) to afford compound 83a (0.11 g, 74%). ¹H NMR (600 MHz, CDCl₃): δ 8.23 (d, J=8.4 Hz, 1H), 7.91 (s, 1H), 7.82 (d, J=7.8 Hz, 1H), 7.78 (d, J=8.4 Hz, 1H), 7.34 (t, J=7.2, Hz, 1H), 7.28 (t, J=7.2 Hz, 1H), 7.08 (t, J=7.8 Hz, 1H), 6.97 (dd, J=6.6, 1.8 Hz, 1H), 6.71 (d, J=7.2, Hz, 1H), 6.63 (d, J=7.8, Hz, 1H), 3.78 (s, 3H), 3.32 (t, J=4.8 Hz, 4H), 3.02-2.94 (m, 2H), 2.88-2.85 (m, 1H), 2.79-2.74 (m, 3H), 2.70 (t, J=4.8 Hz, 4H), 2.56-2.50 (m, 5H), 2.08-2.06 (m, 1H), 1.74 (s, 9H), 1.61-1.54 (m, 1H), 1.52-1.47 (m, 2H), 0.91 (t, J=7.2 Hz, 3H); [α]_{D}²⁵=−28.6 (c=1.0 in CH₂Cl₂).

### (S)-tert-Butyl 3-(4-(2-((5-methoxy-1,2,3,4-tetrahydronaphthalen-2-yl)(propyl)amino)ethyl)piperazin-1-yl)-9H-carbazole-9-carboxylate (83b)

Compound 77b (0.140 g, 0.456 mmol) was reacted with (−)-(5-methoxy-1,2,3,4-tetrahydro-naphthalen-2-yl)-propyl-amine (0.070 g, 0.32 mmol) and NaBH(OAc)₃ (0.151 g, 0.71 mmol) in CH₂Cl₂ (10 mL) according to procedure G. The crude product was purified by silica gel column chromatography with EtOAc to afford compound 83b (0.07 g, 33%). ¹H NMR (600 MHz, CDCl₃): δ 8.18 (d, J=6 Hz, 1H), 8.09 (m, 1H), 7.83 (d, J=7.2 Hz, 1H), 7.38-7.32 (m, 2H), 7.23 (q, J=9.6, 7.5 Hz, 1H), 7.03 (d, J=9 Hz, 1H), 6.99 (t, J=7.8 Hz, 1H), 6.63 (d, J=6.6, Hz, 1H), 6.55 (d, J=7.8, Hz, 1H), 3.70 (s, 3H), 3.18 (t, J=4.2 Hz, 4H), 3.27-3.26 (m, 1H), 3.12-3.10 (m, 2H), 2.93-2.90 (m, 2H), 2.81-2.78 (m, 1H), 2.69-2.64 (m, 5H), 2.48-2.43 (m, 4H), 2.03-2.00 (m, 1H), 1.65 (s, 9H), 1.54-1.47 (m, 1H), 1.45-1.37 (m, 2H), 0.82 (t, J=7.2 Hz, 3H).

### (S)-tert-Butyl 4-(4-(2-((5-methoxy-1,2,3,4-tetrahydronaphthalen-2-yl)(propyl)amino)ethyl)piperazin-1-yl)-9H-carbazole-9-carboxylate (83c)

Compound 77c (0.10 g, 0.25 mmol) was reacted with (−)-(5-methoxy-1,2,3,4-tetrahydro-naphthalen-2-yl)-propyl-amine (0.055 g, 0.25 mmol) and NaBH(OAc)₃ (0.108 g, 0.50 mmol) in CH₂Cl₂ (8 mL) according to procedure G. The crude product was purified by silica gel column chromatography (hexane:EtOAc=1:3) to afford compound 83c (0.124 g, 82%). ¹H NMR (600 MHz, CDCl₃): δ 8.35-8.30 (m, 2H), 7.07 (d, J=7.8 Hz, 1H), 7.44-7.41 (m, 1H), 7.41-7.39 (m, 2H), 7.08 (d, J=7.8 Hz, 1H), 7.04 (d, J=7.8 Hz, 1H), 6.73 (d, J=7.2, Hz, 1H), 6.64 (d, J=8.4 Hz, 1H), 3.79 (s, 3H), 3.53-3.48 (m, 4H), 3.29 (m, 2H), 3.16-3.04 (m, 5H), 2.67-2.60 (m, 1H), 2.37 (bs, 1H), 2.03-2.01 (m, 2H), 1.85-1.84 (m, 3H), 1.75 (s, 9H), 1.26-1.24 (m, 3H), 0.92 (t, J=7.2 Hz, 3H); [α]_{D}²⁵=−20.3 (c=1.0 in CH₂Cl₂).

### Procedure I. 6-((2-(4-(9H-carbazol-2-yl)piperazin-1-yl)ethyl)(propyl)amino)-5,6,7,8-tetrahydronaphthalen-1-ol (84a) (D-654)

A mixture of compound 82a (0.10 g, 0.17 mmol) and 48% aqueous HBr (8 mL) was refluxed at 130° C. for 4 h. The reaction mixture was evaporated to dryness, washed with ether followed by vacuo drying to yield HBr salt of 84a (0.095 g, 78%). ¹H NMR (600 MHz, CD₃OD): δ 8.11 (d, J=8.4 Hz, 1H), 8.03 (d, J=7.8 Hz, 1H), 7.67-7.62 (m, 1H), 7.46 (d, J=7.8 Hz, 1H), 7.38 (t, J=7.2 Hz, 1H), 7.34-7.29 (m, 1H), 7.17 (t, J=7.2 Hz, 1H), 6.96 (t, J=7.8 Hz, 1H), 6.67 (d, J=7.8, Hz, 1H), 6.62 (d, J=7.8, Hz, 1H), 3.99-3.86 (m, 5H), 3.84-3.72 (m, 5H), 3.68-3.63 (m, 2H), 3.39 (m, 1H), 3.33-3.29 (m, 2H), 3.16-3.08 (m, 2H), 2.71-2.66 (m, 1H), 2.63 (m, 1H), 2.49-2.47 (m, 1H), 2.00-1.93 (m, 3H), 1.07 (t, J=7.2 Hz, 3H); ¹³C NMR (150 MHz, CD₃OD): δ 154.74, 140.88, 140.12, 133.27, 129.28, 126.63, 125.85, 122.06, 121.59, 121.12, 120.99, 119.93, 119.78, 119.11, 117.61, 112.08, 110.61, 110.26, 61.26, 52.93, 51.47, 50.73, 50.47, 50.32, 45.16, 29.29, 23.55, 22.35, 18.26, 9.92, 8.31; Anal. Calcd for C₃₁H₃₈N₄O.3HBr: C, 51.33; H, 5.70; N, 7.72. Found: C, 51.50; H, 5.92; N, 7.46.

### 6-((2-(4-(9H-carbazol-3-yl)piperazin-1-yl)ethyl)(propyl)amino)-5,6,7,8-tetrahydro-naphthalen-1-ol (84b) (D-650)

A mixture of compound 82b (0.08 g, 0.13 mmol) and 48% aqueous HBr (7 mL) was reflexed according to procedure I to yield HBr salt of 84b (0.095 g, 98%). ¹H NMR (600 MHz, CD₃OD): δ 8.58 (s, 1H), 8.14 (d, J=7.8 Hz, 1H), 7.76 (d, J=8.4 Hz, 1H), 7.61 (d, J=9.0 Hz, 1H), 7.50 (d, J=7.8 Hz, 1H), 7.44 (t, J=7.2 Hz, 1H), 7.21 (t, J=7.2 Hz, 1H), 6.94 (t, J=7.8 Hz, 1H), 6.66 (d, J=7.8 Hz, 1H), 6.61 (d, J=7.8, Hz, 1H), 4.30 (s, 4H), 4.11-4.04 (m, 6H), 3.93-3.89 (m, 2H), 3.78 (s, 1H), 3.46-3.39 (m, 1H), 3.33-3.29 (m, 2H), 3.22-3.16 (m, 1H), 3.09-3.05 (m, 1H), 2.72-2.69 (m, 1H), 2.52-2.47 (m, 1H), 2.02-1.94 (m, 3H), 1.07 (t, J=7.2 Hz, 3H); ¹³C NMR (150 MHz, CD₃OD): δ 154.67, 141.05, 139.88, 133.32, 132.51, 127.22, 126.31, 123.45, 122.11, 121.60, 120.77, 120.15, 119.76, 117.91, 116.92, 112.40, 112.24, 111.96, 111.54, 111.40, 110.53, 60.95, 53.09, 50.47, 49.91, 29.34, 22.81, 22.37, 18.30, 10.26, 9.73; Anal. Calcd for C₃₁H₃₈N₄O.3HBr.2H₂O: C, 48.90; H, 5.96; N, 7.36. Found: C, 49.30; H, 5.86; N, 7.24.

### 6-((2-(4-(9H-carbazol-4-yl)piperazin-1-yl)ethyl)(propyl)amino)-5,6,7,8-tetrahydro-naphthalen-1-ol (84c) (D-655)

Compound 82c (0.11 g, 0.18 mmol) was refluxed with 48% aqueous HBr (8 mL) according to procedure I to yield HBr salt of 84c (0.097 g, 78%). ¹H NMR (600 MHz, CD₃OD): δ 8.11 (d, J=7.8 Hz, 1H), 8.07 (d, J=7.8 Hz, 1H), 7.46-7.43 (m, 1H), 7.37-7.32 (m, 1H), 7.31-7.27 (m, 1H), 7.25-7.19 (m, 1H), 6.95 (t, J=7.8 Hz, 1H), 6.87 (d, J=7.8 Hz, 1H), 6.79 (d, J=6.6, Hz, 1H), 6.61 (d, J=7.8, Hz, 1H), 4.02 (s, 3H), 3.90-3.85 (m, 5H), 3.58-3.57 (m, 3H), 3.39 (m, 2H), 3.33-3.29 (m, 2H), 3.29-3.17 (m, 2H), 3.11-3.08 (m, 1H), 2.72-2.70 (m, 1H), 2.48 (m, 1H), 1.99-1.94 (m, 3H), 1.07 (t, J=7.2 Hz, 3H); ¹³C NMR (150 MHz, CD₃OD): δ 154.71, 141.47, 139.70, 133.20, 126.61, 125.85, 124.74, 121.97, 121.81, 121.58, 121.25, 119.96, 118.76, 118.66, 115.57, 112.07, 110.24, 110.21, 107.23 61.40, 52.89, 50.25, 48.57, 47.09, 45.57, 43.81, 29.23, 23.57, 22.30, 18.45, 9.91; Anal. Calcd for C₃₁H₃₈N₄O.3HBr: C, 51.33; H, 5.70; N, 7.72. Found: C, 51.07; H, 5.91; N, 8.17.

### (S)-6-((2-(4-(9H-carbazol-2-yl)piperazin-1-yl)ethyl)(propyl)amino)-5,6,7,8-tetrahydronaphthalen-1-ol (85a) (D-653)

Compound 83a (0.10 g, 0.17 mmol) was refluxed with 48% aqueous HBr (8 mL) according to procedure I to yield HBr salt of 85a (0.10 g, 82%). ¹H NMR (600 MHz, CD₃OD): δ 8.11 (d, J=8.4 Hz, 1H), 8.03 (d, J=7.8 Hz, 1H), 7.62 (m, 1H), 7.46 (d, J=7.8 Hz, 1H), 7.38 (t, J=7.2 Hz, 1H), 7.34-7.28 (m, 1H), 7.17 (t, J=7.2 Hz, 1H), 6.96 (t, J=7.8 Hz, 1H), 6.68 (d, J=7.8, Hz, 1H), 6.62 (d, J=7.8, Hz, 1H), 3.99 (s, 3H), 3.93-3.85 (m, 2H), 3.82-3.63 (m, 7H), 3.39 (m, 1H), 3.33-3.29 (m, 2H), 3.17-3.08 (m, 2H), 2.70 (m, 1H), 2.63-2.62 (m, 1H), 2.49-2.47 (m, 1H), 2.01-1.94 (m, 3H), 1.07 (t, J=7.2 Hz, 3H); 13C NMR (150 MHz, CD₃OD): δ 154.73, 140.86, 140.14, 133.28, 129.27, 126.62, 125.82, 122.08, 121.60, 121.09, 120.97, 119.93, 119.76, 119.10, 117.58, 112.08, 110.60, 110.26, 72.29, 61.27, 52.94, 51.83, 51.45, 50.78, 50.49, 41.61, 29.31, 23.48, 22.35, 18.26, 9.92, 8.31; [α]_{D}²⁵=−21.5 (c=1.0 in CH₃OH); Anal. Calcd for C₃₁H₃₈N₄O.3HBr: C, 51.33; H, 5.70; N, 7.72. Found: C, 51.44; H, 5.93; N, 7.47.

### (S)-6-((2-(4-(9H-carbazol-3-yl)piperazin-1-yl)ethyl)(propyl)amino)-5,6,7,8-tetrahydronaphthalen-1-ol (85b) (D-659)

Compound 83b (0.07 g, 0.12 mmol) was refluxed with 48% aqueous HBr (7 mL) according to procedure I to yield HBr salt of 85b (0.085 g, 97%). ¹H NMR (600 MHz, CD₃OD): δ 8.58 (s, 1H), 8.15 (q, J=7.8 Hz, 2H), 7.76 (d, J=6.6 Hz, 1H), 7.61 (d, J=8.4 Hz, 1H), 7.57 (d, J=7.8 Hz, 1H), 7.43 (q, J=7.8 Hz, 1H), 7.21 (d, J=7.8 Hz, 1H), 6.94 (t, J=7.2 Hz, 1H), 6.66 (d, J=7.2 Hz, 1H), 6.61 (d, J=7.8, Hz, 1H), 4.17 (s, 4H), 3.91-3.85 (m, 6H), 3.87-3.85 (m, 2H), 3.57 (s, 1H), 3.50-3.49 (m, 1H), 3.32-3.28 (m, 2H), 3.17-3.14 (m, 1H), 3.08-3.05 (m, 1H), 2.69-2.67 (m, 1H), 2.51-2.46 (m, 1H), 1.96-1.93 (m, 3H), 1.48 (t, J=6.0 Hz, 3H); ¹³C NMR (150 MHz, CD₃OD): δ 157.19, 151.08, 147.936, 147.513.32, 138.93, 137.85, 133.55, 132.78, 129.08, 127.16, 126.92, 122.87, 122.74, 121.64, 119.43, 118.42, 117.40, 107.78, 106.90, 106.54, 57.16, 55.21, 51.85, 50.41, 48.10, 40.131, 29.71, 25.60, 23.87, 22.08, 11.92; [α]_{D}²⁵=−22.2 (c=1.0 in CH₃OH); Anal. Calcd for C₃₁H₃₈N₄O.4HBr: C, 46.18; H, 5.25; N, 6.95. Found: C, 46.13; H, 5.66; N, 7.59.

### (S)-6-((2-(4-(9H-carbazol-4-yl)piperazin-1-yl)ethyl)(propyl)amino)-5,6,7,8-tetrahydronaphthalen-1-ol (85c) (D-656)

Compound 83c (0.112 g, 0.18 mmol) was refluxed with 48% aqueous HBr (9 mL) according to procedure I to yield HBr salt of 85c (0.10 g, 82%). ¹H NMR (600 MHz, CD₃OD): δ 8.09 (d, J=7.2 Hz, 1H), 7.34 (d, J=8.4 Hz, 1H), 7.33 (t, J=7.2 Hz, 1H), 7.26 (dd, J=4.8, 2.4 Hz, 1H), 7.20 (d, J=8.4 Hz, 2H), 6.92 (t, J=7.8 Hz, 1H), 6.72 (d, J=7.2 Hz, 1H), 6.65 (d, J=7.2 Hz, 1H), 6.62 (d, J=7.8, Hz, 1H), 4.02 (s, 3H), 3.90-3.84 (m, 6H), 3.59-3.56 (m, 2H), 3.40 (m, 2H), 3.30-3.28 (m, 2H), 3.19-3.14 (m, 1H), 3.06-3.05 (m, 1H), 2.71-2.68 (m, 1H), 2.47 (bs, 1H), 1.96-1.92 (m, 3H), 1.05 (t, J=7.2 Hz, 3H); ¹³C NMR (150 MHz, CD₃OD): δ 154.69, 141.41, 139.73, 133.24, 127.24, 125.30, 124.28, 122.56, 121.57, 121.21, 120.28, 119.63, 119.28, 118.33, 115.52, 112.43, 111.75, 110.77, 109.71, 107.68, 61.27, 52.94, 51.83, 60.95, 52.82, 50.31, 44.47, 43.68, 22.53, 18.97, 9.92; [α]_{D}²⁵=−17.8 (c=1.0 in CH₃OH); Anal. Calcd for) C₃₁H₃₈N₄O.2HBr. CH₂Cl₂: C, 52.69; H, 5.80; N, 7.68. Found: C, 52.23; H, 6.18; N, 7.96.

### 9-(2-bromoethyl)-9H-carbazole (86)

A suspension of carbazole (1.0 g, 5.98 mmol), K₂CO₃ (1.82 g, 13.16 mmol), tetrabutylammonium bromide (0.039 g, 0.12 mmol) and KOH (2.25 g, 40.07 mmol) in dibromoethane (10 mL, 119.61 mmol) was stirred at 50° C. under N₂ overnight. The reaction mixture was filtered off and diluted with CH₂Cl₂. The organic layer was washed with water, dried over sodium sulfate, filtered and concentrated. The crude product was purified by silica gel column chromatography with petroleum ether to afford compound 86 (0.52 g, 32%). ¹H NMR (400 MHz, CDCl₃): δ 8.10 (d, J=7.6 Hz, 2H), 7.51-7.42 (m, 4H), 7.29-7.25 (m, 2H), 4.71 (t, J=7.2 Hz, 2H), 3.68 (t, J=7.2 Hz, 2H).

### 9-(2-(4-(2-((tert-butyldimethylsilyl)oxy)ethyl)piperazin-1-yl)ethyl)-9H-carbazazole (87)

A mixture of compound 86 (0.7 g, 2.55 mmol), 1-(2-((tert-butyldimethylsilyl)-oxy)ethyl)piperazine (0.75 g, 3.06 mmol), and K₂CO₃ (1.06 g, 7.66 mmol) in acetonitrile (20 mL) was refluxed for 24 h under inert condition. The reaction mixture was cooled to room temperature, filtered, washed with EtOAc and concentrated in vacuo. The crude material was purified by silica gel column chromatography (hexane:EtOAc=3:2) to give compound 87 (0.81 g, 73%). ¹H NMR (400 MHz, CDCl₃): δ 8.09 (d, J=7.2 Hz, 2H), 7.49-7.41 (m, 4H), 7.25-7.21 (m, 2H), 4.45 (t, J=7.2 Hz, 2H), 3.76 (t, J=6.8 Hz, 2H), 2.76 (t, J=7.2 Hz, 2H), 2.68-2.57 (m, 6H), 2.54 (t, J=6.4 Hz, 4H), 0.90 (s, 9H), 0.06 (s, 6H).

### 2-(4-(2-(9H-carbazol-9-yl)ethyl)piperazin-1-yl)ethanol (88)

Compound 97 (0.875 g, 2.0 mmol) was treated with n-tetrabutylammonium fluoride (4.0 mL, 4.0 mmol, 1.0 M solution in THF) in THF (12 mL) according to procedure E. The crude product was purified by silica gel column chromatography (EtOAc:MeOH=5:1) to give compound 88 (0.525 g, 81%). ¹H NMR (400 MHz, CDCl₃): δ 8.09 (d, J=7.2 Hz, 2H), 7.48-7.41 (m, 4H), 7.25-7.21 (m, 2H), 4.44 (t, J=7.2 Hz, 2H), 3.62 (t, J=5.4 Hz, 2H), 2.82-2.76 (m, 4H), 2.61-2.55 (m, 8H).

### 2-(4-(2-(9H-carbazol-9-yl)ethyl)piperazin-1-yl)acetaldehyde (89)

Alcohol 88 (0.30 g, 0.93 mmol) was oxidized using SO₃.py complex (0.74 g, 4.64 mmol), DMSO (3 mL) and Et₃N (0.90 mL, 6.49 mmol) in CH₂Cl₂ (6 mL) according to procedure F. The crude product was purified by silica gel column chromatography (EtOAc:MeOH=9:1) to give aldehyde 89 (0.23 g, 77%). The purified aldehyde was used immediately for next step. ¹H NMR (600 MHz, CDCl₃): δ 9.67 (s, 1H), 8.07 (d, J=7.8 Hz, 2H), 7.47-7.41 (m, 4H), 7.23-7.21 (m, 2H), 4.44-4.42 (m, 2H), 3.38-3.36 (m, 2H), 2.78-2.74 (m, 4H), 2.60-2.56 (m, 6H).

### N⁶-(2-(4-(2-(9H-carbazol-9-yl)ethyl)piperazin-1-yl)ethyl)-N⁶-propyl-4,5,6,7-tetrahydrobenzo[d]thiazole-2,6-diamine (90) (D-626)

Compound 89 (0.14 g, 0.44 mmol) was reacted with (+)-pramipexole (0.083 g, 0.39 mmol) and NaBH(OAc)₃ (0.185 g, 0.87 mmol) in CH₂Cl₂ (10 mL) according to procedure G. The crude product was purified by silica gel column chromatography (EtOAc:MeOH=4:1) to afford compound 90 (0.115 g, 57%). The compound was converted to HCl salt. ¹H NMR (400 MHz, CDCl₃): δ 8.07 (d, J=8.4 Hz, 2H), 7.47-7.39 (m, 4H), 7.23-7.20 (m, 2H), 5.20 (bs, 2H), 4.41 (t, J=7.2 Hz, 2H), 3.04-2.97 (m, 1H), 2.75 (t, J=7.2 Hz, 2H), 2.69-2.48 (m, 14H), 2.45 (t, J=7.2 Hz, 4H), 1.97-1.94 (m, 1H), 1.73-1.63 (m, 1H), 1.49-1.40 (m, 2H), 0.87 (t, J=7.2 Hz, 3H); ¹³C NMR (100 MHz, CDCl₃): δ 166.01, 144.77, 140.29, 125.70, 122.90, 120.39, 118.96, 116.88, 108.54, 58.52, 58.08, 56.12, 53.59, 53.48, 48.28, 40.94, 26.46, 25.82, 25.09, 22.33, 11.85; Anal. Calcd for C₃₀H₄₀N₆S.5HCl.CH₃OH: C, 50.25; H, 6.61; N, 11.72. Found: C, 50.81; H, 7.00; N, 11.33.

### (S)—N⁶-(2-(4-(2-(9H-carbazol-9-yl)ethyl)piperazin-1-yl)ethyl)-N⁶-propyl-4,5,6,7-tetrahydrobenzo[d]thiazole-2,6-diamine (91) (D-637)

Compound 89 (0.19 g, 0.59 mmol) was reacted with (−)-pramipexole (0.112 g, 0.53 mmol) and NaBH(OAc)₃ (0.25 g, 1.18 mmol) in CH₂Cl₂ (12 mL) according to procedure G. The crude product was purified by silica gel column chromatography (EtOAc:MeOH=4:1) to afford compound 91 (0.13 g, 47%). The compound was converted to HCl salt. ¹H NMR (600 MHz, CDCl₃): δ 8.07 (d, J=7.8 Hz, 2H), 7.45 (t, J=7.8 Hz, 2H), 7.40 (d, J=7.8 Hz, 2H), 7.22 (t, J=7.8 Hz, 2H), 5.11 (bs, 2H), 4.41 (t, J=7.2 Hz, 2H), 3.03-2.98 (m, 1H), 2.74 (t, J=7.2 Hz, 2H), 2.69-2.48 (m, 14H), 2.45 (t, J=7.2 Hz, 4H), 1.96-1.94 (m, 1H), 1.71-1.64 (m, 1H), 1.48-1.41 (m, 2H), 0.87 (t, J=7.2 Hz, 3H); ¹³C NMR (150 MHz, CDCl₃): δ 165.85, 144.91, 140.28, 125.68, 122.89, 120.39, 118.95, 117.00, 108.53, 58.58, 58.08, 56.13, 53.63, 53.52, 48.33, 40.95, 26.52, 25.84, 25.10, 22.35, 11.85; [α]_{D}²⁵=−19.8 (c=1.0 in CH₂Cl₂); Anal. Calcd for C₃₀H₄₀N₆S.4HCl.2CH₃OH: C, 52.89; H, 7.21; N, 11.57. Found: C, 53.16; H, 7.14; N, 11.68.

Evaluation of Binding Affinity and Functional Potencies at Dopamine D2 and D3 Receptors.

Binding affinity was evaluated by inhibition of [³H]spiroperidol (15.0 Ci/mmol, Perkin-Elmer) binding to DA rD₂ and rD₃ receptors expressed in HEK-293 cells in a buffer containing 0.9% NaCl. Functional activity of test compounds in activating dopamine hD₂ and hD₃ receptors expressed in CHO cells was measured by stimulation of [³⁵S]GTPγS (1250 Ci/mmol, Perkin-Elmer) binding in comparison to stimulation by the full agonist DA. All these procedures were described by us previously.

In Vivo Study of (−)-81b, (−)-85a and (−)-85c in Parkinsonian Rats

Drugs and Chemicals.

The following commercially available drug was used in the experiment: reserpine hydrochloride (Alfa Aesar). The TFA salt of (−)-81b (D-636) and HBr salts of (−)-85a (D-653) and (−)-85c (D-656) were dissolved in water. Reserpine was dissolved in 20 μL of glacial acetic acid and further diluted with 5.5% glucose solution. The compounds for this study were administered in a volume of 0.1-0.2 mL for subcutaneous administration and 0.5-0.7 ml for interaperitoneal administration into each rat.

Animals.

In rodent studies, animals were male and female Sprague-Dawley rats from Harlan (Indianapolis, Ind.) weighing 220-225 g unless otherwise specified. Animals were maintained in sawdust-lined cages in a temperature and humidity controlled environment at 22±1° C. and 60±5% humidity, respectively. A 12 h light/dark cycle was maintained, with lights on from 6:00 a.m. to 6:00 p.m. They were group-housed with unrestricted access to food and water. All experiments were performed during the light component. All animal use procedures were in compliance with the Wayne State University Animal Investigation Committee, consistent with AALAC guidelines.

Reversal of Reserpine-Induced Hypolocomotion in Rats.

The ability of compounds (−)-81b, (−)-85a and (−)-85c to reverse reserpine-induced hypolocomotion was investigated according to a reported procedure. Reserpine (5.0 mg/kg, sc) was administered 18 h before the injection of drug or vehicle. The rats were placed individually in the chambers for 1 h for acclimatization before administration of the test drugs or vehicle. Immediately after administration of drug or vehicle, animals were individually placed in Opto-Varimex 4 animal activity monitor chamber (Columbus Instruments, Ohio, USA) to start measuring locomotor activity. Locomotion was monitored for 6 h. Consecutive interruption of two infrared beams, situated 50 cm apart and 4 cm above the cage floor, in the monitor chamber recorded movement. The data were presented as horizontal activity (HACTV). The effect of individual doses of drugs on locomotor activity was compared with respect to saline treated controls (mean±SEM). The data were analyzed by one-way analysis of variance (ANOVA) followed by Dunnett's post hoc test. The effect was considered significant if the difference from control group was observed at p<0.05.

Assessment of Anti-Oxidant Activity:

Cell Cultures and Treatments. PC12 cells (ATCC CRL1721.1, Manassas, Va., USA), a rat adrenal pheochromocytoma cell line, were cultured in T-75 flasks (Greiner Bio One, Frickenhausen, Germany) and maintained in RPMI 1640 medium supplemented with 10% heat inactivated horse serum, 5% fetal bovine serum, 100 U/ml penicillin, and 100 μg/mL streptomycin at 37° C. in 95% air/5% CO₂. Stock solutions of (−)-81b, (−)-85a and (−)-85c were prepared in dimethylsulfoxide (DMSO) and stored at −20° C., a stock solution of 6-Hydroxydopamine (6-OHDA) was stored at −80° C., a solution of 6-carboxy-2′,7′-dichlorodihydroflourescence diacetate (carboxy-DCFDA) was prepared fresh in DMSO before addition. All stock solution were stored for the period of the experiments.

Measurement of Antioxidant Activity:

To determine the effects of (−)-81b, (−)-85a and (−)-85c in decreasing reactive oxygen species (ROS) in PC12 cells produced by the neurotoxin 6-OHDA, a quantitative fluorometric ROS assay was performed. PC12 cells were plated at 30,000 cells/well density in 100 μL media in 96-well black plates and incubated at 37° C. under 5% CO₂ atmosphere for 24 h. The cells were treated for 24 h with various concentrations of compounds (−)-81b, (−)-85a and (−)-85c. Then the drugs containing media were removed and replaced with DCFDA 20 μm for 30 min in an incubator (37° C., 5% CO₂). The DCFDA containing media was then removed and the cells were washed with PBS buffer to remove the traces of the dye. Fresh culture media was added followed by treatment with 75 μM of 6-OHDA alone for an additional 1 h under the same conditions. After incubation for 1 h, the fluorescence was measured using spectrophotometer fluorescence generated microplate reader (Biotek Epoch, Winooski, Vt., USA) at excitation 497 nm and emission at 527 nm. Data from at least three experiments were analyzed by one-way analysis of variance (ANOVA) followed by Tukey's multiple comparison post hoc test using GraphPad software (version 6, San Diego, Calif., USA). The specific fluorescence emission was calculated after subtraction of the DCFDA untreated control cells from the DCFDA treated cells for both the background and 1 h treatment conditions. This was followed by the division of the 1 h treatment data by the background activity which was determined from vials before treatment with 6-OHDA, to derive the final data point.

Assessment of Cell Viability:

Cell Cultures and Treatments.

PC12 cells (ATCC CRL1721.1, Manassas, Va., USA), a rat adrenal pheochromocytoma cell line, were cultured in T-75 flasks (Greiner Bio One, Frickenhausen, Germany) and maintained in RPMI 1640 medium supplemented with 10% heat inactivated horse serum, 5% fetal bovine serum, 100 U/ml penicillin, and 100 μg/mL streptomycin at 37° C. in 95% air/5% CO₂. Stock solutions of (−)-81b, (−)-85a, (−)-85c, and 6-hydroxydopamine (6-OHDA) were prepared in dimethylsulfoxide (DMSO) and aliquots were stored at −20° C. and −80° C., respectively. For all experiments assessing neuroprotective effects of the test compounds, PC12 cells were pretreated with indicated concentrations of (−)-81b, (−)-85a and (−)-85c for 24 h and then treated with 75 μM 6-OHDA for another 24 h. The control cells were treated with above media containing 0.01% DMSO only.

To determine the neuroprotective effects of (−)-81b, (−)-85a and (−)-85c in the presence of neurotoxin 6-OHDA, a quantitative colorimetric MTT assay was performed. PC12 cells were plated at 17000 cells/well density in 100 μL of media in 96-well plates and incubated at 37° C. under 5% CO₂ atmosphere for 24 h. Cells were treated with varying concentrations of the test compounds to determine their direct effect on cell viability. Neuroprotection experiments were conducted by treating cells for 24 h with varying concentrations of (−)-81b, (−)-85a and (−)-85c. Then the drug containing media was replaced with fresh culture media followed by treatment with 75 μM of 6-OHDA alone for an additional 24 h under the same condition. After incubation for 24 h, 5 mg/mL MTT solution (prepared in Dulbecco's phosphate-buffered saline) was added to the cells (to a final concentration of 0.5 mg/mL) and the plates were further incubated at 37° C. in 95% air/5% CO₂ atmosphere for 3-4 h to produce dark-blue formazan crystals. Afterward, the plates were centrifuged at 450 g for 10 min and the supernatants were carefully removed. Formazan crystals were dissolved by adding 100 μL of methanol:DMSO (1:1) mixture to each well and shaking gently at 400 rpm for 30 min at room temperature on a Thermomix R shaker (Eppendorf, Hamburg, Germany). The absorbance was measured on a microplate reader (Biotek Epoch, Winooski, Vt., USA) at 570 nm with background correction performed at 690 nm. Data from at least three experiments were analyzed using GraphPad software (version 6, San Diego, Calif., USA). Cell viability was defined as percentage reduction in absorbance compared to untreated controls. The data were analyzed by one-way analysis of variance (ANOVA) followed by Tukey's multiple comparison post hoc test.

Assessment of α-Synuclein Aggregation Inhibition Activity:

The experimental conditions were followed as described in our earlier publication. Briefly, α-syn fibrils were formed by incubating purified α-syn at a concentration of 5 mg/mL at 37° C. under constant agitation at 1000 rpm in a Thermomix R shaker (Eppendorf, Hamburg, Germany) for a period of 5 days. These fibrils at a concentration of 0.5% were used to seed the aggregation of α-syn at 1.25 mg/ml (86.45 μM) with or without compounds D636 or D653 at a concentration of 172.9 μM without agitation at 37° C. for a period of 30 days. Samples were collected at OD and 30D. Thioflavin T (ThT) assay was performed using these samples as described previously to evaluate the extent of aggregation.

Further, the cytotoxicity of the aggregates formed was evaluated in PC12 cells. Briefly, PC12 cells were treated with α-syn aggregates such that its concentration is 10 μM. 24 h following treatments, viability was measured by MTT assay as described previously.

### Additional Compounds

### 93. (2-Bromo-ethoxy)-tert-butyl-dimethyl-silane

A solution of bromoethanol 92 (1 mL, 14.1 mmol), imidazole (1.9 g, 27.9 mmol), and tert-butyldimethylsilyl chloride (2.1 g, 13.9 mmol) in CH₂Cl₂ (15 mL) was stirred at room temperature for 20 h. After the reaction was complete, water (20 mL) was added, and reaction mixture was extracted with CH₂Cl₂ (3×50 mL). The CH₂Cl₂ layer was dried over Na₂SO₄, filtered, and concentrated in vacuo. The resulting crude was purified by column chromatography using 5% ethyl acetate in hexanes to give compound 93 (2.85 g, 86%). ¹H NMR (CDCl₃, 600 MHz): δ ppm 0.09 (s, 6H), 0.91 (s, 9H), 3.40 (t, J=6.6 Hz, 2H), 3.89 (t, J=6.6 Hz, 2H).

### 95. 1-[2-(tert-Butyl-dimethyl-silanyloxy)-ethyl]-piperazine

A suspension of piperazine 94 (2.3 g, 26.7 mmol), intermediate 93 (2.55 g, 10.7 mmol), and potassium carbonate (11 g, 79.6 mmol) in acetonitrile (35 mL) was refluxed at 80-90° C. for overnight. The reaction mixture was filtered, and the filtrate was condensed in vacuo. The residue was then diluted with ether, washed with water, dried over Na₂SO₄, filtered, and concentrated to produce intermediate 95 (2.19 g, 84%). ¹H NMR (CDCl₃, 600 MHz): δ ppm 0.06 (s, 6H), 0.89 (s, 9H), 2.49-2.53 (m, 6H), 2.89 (t, J=4.8 Hz, 4H), 3.76 (t, J=6.6 Hz, 2H).

### 98. 4′-bromo-3,4-dimethoxy-1,1′-biphenyl

Into a mixture of starting material 96 (5.96 g, 25.3 mmol), Pd(PPh₃)₄ (0.73 g, 0.63 mmol), and K₂CO₃ (3.5 g, 25.3 mmol) in toluene/H₂O (20 mL/10 mL), a suspension of boronic acid 97 (2.3 g, 12.6 mmol) in ethanol (10 mL) was added and stirred at room temperature for 10 min. The reaction mixture was refluxed at 85-90° C. for 3 h. The solvent was partially evaporated, and the mixture was extracted with ethyl acetate (3×100 mL), which was then concentrated in vacuo after dried over Na₂SO₄. The resulting crude was purified by column chromatography using 5-10% ethyl acetate in hexanes to give compound 98 (2.89 g, 78%). ¹H NMR (CDCl₃, 600 MHz): δ ppm 3.93 (s, 3H), 3.95 (s, 3H), 6.94 (d, J=8.4 Hz, 1H), 7.06 (d, J=1.8 Hz, 1H), 7.11 (dd, J=8.4, 2.4 Hz, 1H), 7.42-7.43 (m, 2H), 7.54 (dt, J=8.4, 1.8 Hz, 2H).

### 99. 1-(2-((tert-butyldimethylsilyl)oxy)ethyl)-4-(3′,4′-dimethoxy-[1,1′-biphenyl]-4-yl)piperazine

The mixture of intermediate 95 (3.33 g, 13.6 mmol), intermediate 98 (2 g, 6.8 mmol), BINAP (0.425 g, 0.68 mmol), and Cs₂CO₃ (6.67 g, 20.5 mmol) in toluene (60 mL) was degassed by bubbling N₂ for 5 min. Then Pd(OAc)₂ (115 mg, 0.51 mmol) was added quickly followed by degassing for another 5 min. The reaction mixture was refluxed at 110° C. for 24 h under inert condition. Afterward, it was cooled to room temperature, filtered through a pad of celite, washed with CH₂Cl₂, and concentrated in vacuo. The resulting crude was purified by column chromatography using 10-30% ethyl acetate in hexanes to give compound 99 (2.4 g, 77%). ¹H NMR (CDCl₃, 600 MHz): δ ppm 0.80 (s, 6H), 0.91 (s, 9H), 2.60 (t, J=6.6 Hz, 2H), 2.71 (t, J=4.8 Hz, 4H), 3.24 (t, J=4.8 Hz, 4H), 3.81 (t, J=5.4 Hz, 2H), 3.91 (s, 3H), 3.94 (s, 3H), 6.91 (d, J=7.8 Hz, 1H), 6.97 (d, J=8.4 Hz, 2H), 7.07-7.11 (m, 2H), 7.46 (d, J=7.8 Hz, 2H).

### 100. 2-(4-(3′,4′-dimethoxy-[1,1′-biphenyl]-4-yl)piperazin-1-yl)ethanol

Compound 99 (2 g, 4.4 mmol) was dissolved in THF (25 mL) and cooled to 0° C. TBAF (n-tetrabutylammonium fluoride) 1M in THF (8.76 mL, 8.76 mmol) was added at 0° C., and reaction stirred at room temperature for 2 h. After reaction was complete, saturated NaHCO₃ solution (50 mL) was added, and reaction mixture was extracted with CH₂Cl₂ (3×150 mL), which was then concentrated in vacuo. The resulting crude was purified by column chromatography using 0-5% methanol in CH₂Cl₂ to give compound 100 (1.34 g, 89%). ¹H NMR (CDCl₃, 600 MHz): δ ppm 2.63 (t, J=5.4 Hz, 2H), 2.71 (t, J=5.4 Hz, 4H), 3.26 (t, J=4.8 Hz, 4H), 3.68 (t, J=5.4 Hz, 2H), 3.92 (s, 3H), 3.94 (s, 3H), 6.92 (d, J=8.4 Hz, 1H), 6.86 (d, J=9.0 Hz, 2H), 7.07-7.11 (m, 2H), 7.47 (d, J=9.0 Hz, 2H).

### 101. 2-(4-(3′,4′-dimethoxy-[1,1′-biphenyl]-4-yl)piperazin-1-yl)acetaldehyde

A solution of DMSO (0.31 mL, 4.37 mmol) in CH₂Cl₂ (2 mL) was added into a round-bottom flask containing a stirring solution of oxalyl chloride (0.21 mL, 2.41 mmol) in CH₂Cl₂ (2 mL) at −78° C. The mixture was stirred for 0.5 h at the same temperature, and compound 100 (0.42 g, 1.23 mmol) in CH₂Cl₂ (3 mL) was added. The stirring of the reaction mixture was continued at −78° C. for another 0.5 h. Thereafter, Et₃N (1.38 mL, 9.90 mmol) was added, and the reaction was allowed to warm to room temperature and continued for another 1 h. The reaction mixture was quenched by the addition of a saturated solution of NaHCO₃ at 0° C. and was extracted with CH₂Cl₂ (3×100 mL). The combined organic layer was dried over Na₂SO₄, and the solvent was removed in vacuo to produce the crude, which was purified by column chromatography using ethyl acetate to give compound 101 (0.379 g, 91%). ¹H NMR (600 MHz, CDCl₃): δ ppm 2.73 (t, J=4.8 Hz, 4H), 3.27 (s, 2H), 3.30 (t, J=4.8 Hz, 4H), 3.92 (s, 3H), 3.94 (s, 3H), 6.92 (d, J=8.4 Hz, 1H), 6.98 (d, J=9.0 Hz, 2H), 7.07-7.11 (m, 2H), 7.47 (d, J=9.0 Hz, 2H), 9.76 (s, 1H).

### Procedure A. 103a. 2-(4-(3′,4′-dimethoxy-[1,1′-biphenyl]-4-yl)piperazin-1-yl)-N,N-diethylethanamine

Into a stirring solution of amine 102a (38.4 μL, 0.37 mmol) in CH₂Cl₂ (10 mL) was added aldehyde 101 (127 mg, 0.37 mmol), and the mixture stirred for 1.5 h. NaBH(OAc)₃ (0.158 g, 0.75 mmol) was then added portion-wise. The reaction was stirred for 48 h at room temperature. The reaction mixture was quenched with a sat. NaHCO₃ solution at 0° C. and extracted with CH₂Cl₂ (3×30 mL). The combined organic layer was dried over Na₂SO₄, and solvent was removed under reduced pressure. The crude product was purified by column chromatography using 0-10% MeOH in CH₂Cl₂ to give compound 103a (127 mg, 86%). ¹H NMR (600 MHz, CDCl₃): δ ppm 1.08 (t, J=7.2 Hz, 6H), 2.59-2.73 (m, 12H), 3.25 (t, J=5.4 Hz, 4H), 3.91 (s, 3H), 3.94 (s, 3H), 6.91 (d, J=8.4 Hz, 1H), 6.96 (d, J=8.4 Hz, 2H), 7.07-7.11 (m, 2H), 7.46 (d, J=9.0 Hz, 2H).

### 103b. N-(2-(4-(3′,4′-dimethoxy-[1,1′-biphenyl]-4-yl)piperazin-1-yl)ethyl)-N-isopropylpropan-2-amine

Aldehyde 101 (194 mg, 0.57 mmol), amine 102b (80.5 μL, 0.57 mmol), and NaBH(OAc)₃ (0.242 g, 1.14 mmol) in CH₂Cl₂ (10 mL) were reacted using procedure A, and the resulting crude was purified by column chromatography using 0-10% MeOH in CH₂Cl₂ to give compound 103b (117 mg, 48%). ¹H NMR (600 MHz, CDCl₃): δ ppm 1.08 (bs, 12H), 2.45-2.81 (m, 8H), 2.98-3.16 (m, 2H), 3.25 (t, J=4.2 Hz, 4H), 3.92 (s, 3H), 3.94 (s, 3H), 6.91 (d, J=8.4 Hz, 1H), 6.97 (d, J=9.0 Hz, 2H), 7.07-7.10 (m, 2H), 7.46 (d, J=8.4 Hz, 2H).

### 103c. 2-(4-(3′,4′-dimethoxy-[1,1′-biphenyl]-4-yl)piperazin-1-yl)-N-(4-fluorobenzyl)ethanamine

Aldehyde 101 (185 mg, 0.54 mmol), amine 102c (61.7 μL, 0.54 mmol), and NaBH(OAc)₃ (0.23 g, 1.09 mmol) in CH₂Cl₂ (10 mL) were reacted using procedure A, and the resulting crude was purified by column chromatography using 0-10% MeOH in CH₂Cl₂ to give compound 103c (73 mg, 30%). ¹H NMR (600 MHz, CDCl₃): δ ppm 2.59 (t, J=6.0 Hz, 6H), 2.75 (t, J=6.6 Hz, 2H), 3.22 (t, J=4.8 Hz, 4H), 3.81 (s, 2H), 3.91 (s, 3H), 3.94 (s, 3H), 6.91 (d, J=8.4 Hz, 1H), 6.97 (d, J=8.4 Hz, 2H), 7.02 (t, J=8.4 Hz, 2H), 7.07-7.11 (m, 2H), 7.31 (dd, J=8.4, 5.4 Hz, 2H), 7.46 (d, J=8.4 Hz, 2H).

### Procedure B. 104a (D-690). 4′-[4-(2-Diethylamino-ethyl)-piperazin-1-yl]-biphenyl-3,4-diol (D-690)

Compound 103a (44 mg, 0.11 mmol) was treated with 48% hydrobromic acid (2.5 mL) and refluxed for 6 h. HBr was removed to obtain the residue, which was then dissolved in MeOH and filtered through cotton. The filtrate was concentrated and washed with ether for three times followed drying to give the hydrobromide salt of compound 104a (D-690) (63.5 mg, 89%). ¹H NMR (600 MHz, CD₃OD): δ ppm 1.40 (t, J=7.2 Hz, 6H), 3.31-3.94 (m, 16H), 6.78 (d, J=7.8 Hz, 1H), 6.91 (d, J=8.4 Hz, 1H), 7.00 (d, J=2.4 Hz, 1H), 7.10-7.31 (m, 2H), 7.52 (d, J=8.4 Hz, 2H). Mp 198-200° C. Ana. (C₂₂H₃₈Br₃N₃O₄) C, H, N.

### 104b (D-687). 4′-[4-(2-Diisopropylamino-ethyl)-piperazin-1-yl]-biphenyl-3,4-diol (D-687)

Compound 103b (41 mg, 0.096 mmol) was treated with 48% hydrobromic acid (2.2 mL) and refluxed for 6 h by following procedure B. The crude was washed with ether for three times followed drying to give the hydrobromide salt of compound 104b (D-687) (49.6 mg, 75%). ¹H NMR (600 MHz, CD₃OD): δ ppm 1.46 (s, 6H), 1.47 (s, 6H), 3.34-3.90 (m, 14H), 6.79 (d, J=7.8 Hz, 1H), 6.90 (d, J=8.4 Hz, 1H), 7.00 (s, 1H), 7.18-7.28 (m, 2H), 7.51 (d, J=8.4 Hz, 2H). Mp 168-171° C. Ana. (C₂₄H₄₃Br₃N₃O_{4.5}) C, H, N.

### 104c (D-688). 4′-{4-[2-(4-Fluoro-benzylamino)-ethyl]-piperazin-1-yl}-biphenyl-3,4-diol (D-688)

Compound 103c (63 mg, 0.14 mmol) was treated with 48% hydrobromic acid (3.2 mL) and refluxed for 6 h by following procedure B. The crude was washed with ether for three times followed drying to give the hydrobromide salt of compound 104c (D-688) (80.5 mg, 82%). ¹H NMR (600 MHz, CD₃OD): δ ppm 3.59-3.72 (m, 12H), 4.39 (s, 2H), 6.82 (d, J=7.8 Hz, 1H), 6.93 (d, J=6.6 Hz, 1H), 7.03 (d, J=1.8 Hz, 1H), 7.20-7.25 (m, 4H), 7.53 (d, J=9.0 Hz, 2H), 7.69-7.71 (m, 2H). Mp 200-203° C. Ana. (C₂₅H₃₅Br₃FN₃O₄) C, H, N.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A compound having formula I for treating a neurodegenerative and other related CNS diseases: 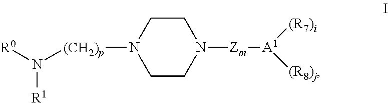
or a pharmaceutically acceptable salt or ester thereof,
wherein
R⁰ is an optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, C₆₋₁₀ aryl, or 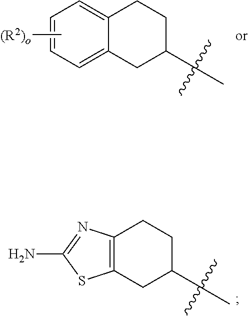
R¹ is an optionally substituted C₁₋₈ alkyl, C₁₋₈ alkoxyl, C₂₋₈ alkenyl, C₂₋₈ alkynyl, C₄₋₈ cycloalkyl, C₄₋₈ cycloalkenyl, or C₆₋₁₀ aryl;
R² are hydroxyl, C₁₋₄ alkyl, C₁₋₄ alkoxyl, C₁₋₈ alkyl, C₂₋₈ alkenyl, C₂₋₈ alkynyl, C₄₋₈ cycloalkyl, C₄₋₈ cycloalkenyl; C₆₋₁₀ aryl, —NR³_{q} or C₁₋₁₀ hydrocarbon groups optionally containing one or more O, N, S, or Se heteroatoms where R³ individually are H or organyl groups and q is 2 or 3, with the proviso that when q is 3, the group bears a positive formal charge;
R⁷, R⁸ are each independently, H, hydroxyl, oxo, C₁₋₈ alkyl, C₁₋₈ alkoxyl, C₂₋₈ alkenyl, C₂₋₁₀ alkynyl, C₅₋₇ cycloalkyl, C₅₋₇ cycloalkenyl, halo, C₁₋₄ aldehyde, or —NR⁴_{q} where R⁴ is H, C₁₋₈ alkyl, C₂₋₈ alkenyl, C₄₋₈ cycloalkyl, 3H-1,2-dithiole-3-thione, C₄₋₈ cycloalkenyl, or C₆₋₁₀ aryl;
A¹ is a C₆₋₁₂ aryl group, C₅₋₁₂ heteroaryl group, substituted 3-hydroxypyridin-4(1H)-one, 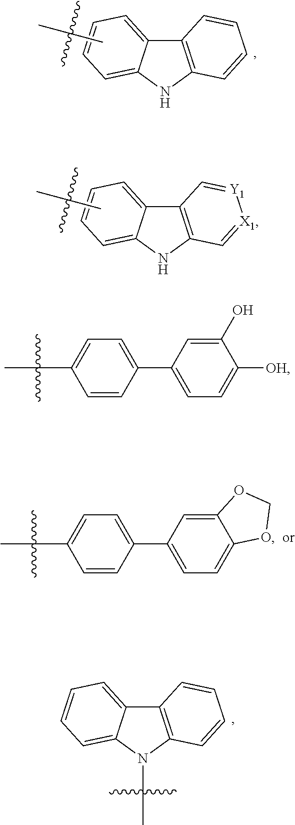
with i hydrogen atoms replaced with R⁷ and j hydrogen atoms replaced with R⁸;
p is an integer from 1 to 6;
X¹, Y¹ are each independently CH or N;
Zₘ is absent or a divalent linking moiety in which Z is repeated m times;
m is an integer from 0 to 5;
i, j are each independently 0, 1, 2, or 3; and
o is 0, 1, 2, 3, or 4.

2. The compound of claim 1 wherein A¹ is:

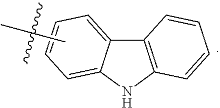

3. The compound of claim 1 wherein A¹ is:

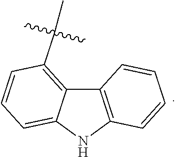 [SMILES: *c1cccc2[nH]c3ccccc3c12]

4. The compound of claim I wherein A¹ is:

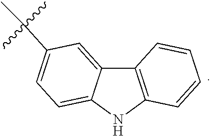 [SMILES: *c1ccc2[nH]c3ccccc3c2c1]

5. The compound of claim 1 wherein A¹ is:

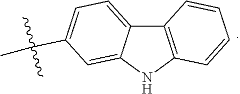 [SMILES: *c1ccc2c(c1)[nH]c1ccccc12]

6. The compound of claim 1 wherein A¹ is:

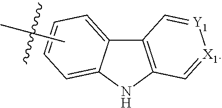

7. The compound of claim 1 wherein A¹ is:

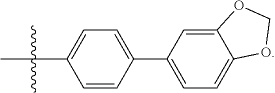 [SMILES: *c1ccc(-c2ccc3c(c2)OCO3)cc1]

8. The compound of claim 1 wherein R¹ is substituted with a component selected from the group consisting of C₁₋₈ alkoxy, C₁₋₄ acyloxy, C₁₋₄ acyl, —C(O)—R⁴, and —R⁵—NH—SO₂—NR⁴ᵣ where —R⁵—NH—C(O)—R⁴; —R⁵—NR⁴ᵣ, or —R⁵—Ar where
R⁴ is H, C₁₋₈ alkyl, C₂₋₈ alkenyl, C₄₋₈ cycloalkyl, C₄₋₈ cycloalkenyl, or C₆₋₁₀ aryl;
R⁵ is C₂₋₈ alkenyl;
r is 2 or 3; and
Ar is a C₆₋₁₀ aryl ring system, optionally including one or more heteroatoms or C₅₋₁₀ heteroaryl; with the proviso that when r is 3, the nitrogen of the NR⁴ᵣ group will bear a positive formal charge.

9. The compound of claim 8 wherein Ar is an optionally substituted phenyl, thienyl, pyridyl, bipyridyl, biphenylyl, or naphthyl.

10. The compound of claim 1 wherein R² are hydroxyl, C₁₋₈ alkyl, C₁₋₈ alkoxyl, C₂₋₈ alkenyl, C₂₋₈ alkynyl, C₄₋₈ cycloalkyl, C₄₋₈ cycloalkenyl; C₆₋₁₀ aryl, —NR³_{q} —NH—C(O)—R³, or —NH—C(O)—NR⁴₂, where R³ individually are H, C₁₋₈ alkyl, C₂₋₈ alkenyl, C₂₋₁₀ alkynyl, C₄₋₈ cycloalkyl, C₄₋₈ cycloalkenyl, or C₆₋₁₀ aryl where q is 2 or 3, with the proviso that when q is 3, the group bears a positive formal charge and wherein the hydrocarbon groups in each case are optionally substituted with —CN, C₁₋₈ alkyl, —OR³, —OH, halo, or —CF₃.

11. The compound of claim 1 wherein A¹ is directly bonded to a nitrogen atom of the piperazinyl group.

12. The compound of claim 1 wherein A¹ is a C₆₋₁₂ aryl group consisting of 1 to 4 rings or a C₅₋₁₂ heteroaryl optionally linked, and optionally substituted by C₁₋₄ alkyl, C₂₋₄ alkenyl, C₂₋₁₀ alkynyl, C₅₋₇ cycloalkyl, C₅₋₇ cycloalkenyl, halo, C₁₋₄ aldehyde, or —NR⁴_{q}, where R⁴ is C₁₋₈ alkyl, OH, C₁₋₈ alkoxyl, C₂₋₈ alkenyl, C₄₋₈ cycloalkyl, C₄₋₈ cycloalkenyl, or C₆₋₁₀ aryl; and q is 2 or 3, with the proviso that when q is 3, the group bears a positive formal charge.

13. The compound of claim 1 wherein A¹ is an optionally substituted thienyl, pyridinyl, phenyl, biphenyl, naphthyl, quinolone, indole, or isoquinoline.

14. The compound of claim 9 wherein A¹ is substituted with C₁₋₈ alkyl, —CN, halo, OH, C₁₋₈ alkoxyl, NH₂SO₂R³, CF₃, arylsulfonyl, arylsulfonamide, o-OCH₃, 2,3-dichloro, pyridinyl, bipyridinyl, or p-NHSO₂CH₃.

15. The compound of claim 9 wherein Z is —CH₂—, —CO—, —N—CH₂— or —N—CO—.

16. The compound of claim 1 wherein A¹(R₇)ᵢ(R₈)ⱼ is:

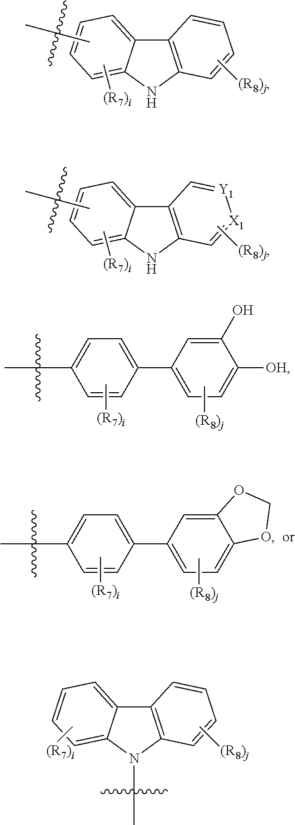

17. The compound of claim 1 wherein Z is selected from the group consisting of —CH₂—, —CHOHCH₂—, —CHOHCH₂CH₂—, —CHOHCH₂CH₂CH₂—, —CO—, —N—CH₂—, —N—CO—, —(CH₂)ₙ—, —CHOH(CH₂)ₙ—, —(CH₂)ₙCO—, —(CH₂)ₙNCO(CH₂)ₖ—, C₁₋₁₀ carboximido, C₁₋₁₀ alkanediyl, C₂₋₁₀ alkanediyl, C₂₋₁₀ alkynediyl, and combinations thereof; m is 1, 2, 3, 4, or 5, and n and k are each independently integers 0, 1, 2, 3, 4, 5, 6, 7, or 8.
